SEQUENCE LISTING (87 sequences)

SEQ ID NO: 1 - protein, 1105 aa, Brevibacillus laterosporus
MFFLICDPAKGGETRLQDSGWKKKETTSNKISQNQLLYIRGGEPKVMKKVVNSLLASALALSVVPVVAGAEEVKNTGTEETSTTAPQLDPAMEKVVKRLSALGLAAGDDKGNFNLDQTITRSEFATLMVRARGLEEAAKTATFNPFFKDVIATDWFQGFVSIAVSQDLAKGYPDKTFKPKNSVTYAEAATMLVRALGYEPLALQKGTWPNNFISQASELGISQGVTIDANKPAVRGDVFKMLDNSLRIDLMKQTTVGSQNNHEVLKGKNLLNEYLKVQVYDTKWTMTDRNKPEDLPSVSNVPVIGLGSLKYNEVELTSDSRSFFNKVKYKVADSINANDYAGQRVQVWIKDRDNTVVWMEGSEDETVVFDKYSALHISGKKLSPTKDKDQIKKDKFNKLTLELDSGTKYDFAEDVSVTFNFERATKASDVQEMLQKVLEADDAYSVKVVLNTKNEIRYISIVDDRSGDQNWVNNHFGSELIKKVTDKKITTLVGKDLALEDKKEGIDYLVFLDGKPAKLADLKEMDVYHAYRADGNKDKLLIFANRSTKEGKVEKVRSKSESDYRIVIDGKEYRFRNASFAKDKDKEIEDQITPDDIDRLEGTKVKVYLDAVGRIRHIEGYEANDRKQMAIISRSGTYDPYSDQYTFKVYTEKGNSKTVKLDGKYVKNEEGEKFSDLKKNESEKKIAEKYLTLSKDKNFILAQIELDASGDIEKVELKDTSKAKHMSKDQWKKDSDKDDETVKFGGKTYDFSSSSVVFDGSKGIQSGDVTSLKSPGTIKWSKIAEKEAEVYFTVDGSDVEAVFVVDSKGIASGSEYGVVVEAYRSSGVDKLKIKHRDDDGKWVEKEYKYDGDGKDKLEKTFIRFSADSDDLIDEDDVRVIVDARSDDEYNYKILKEKDYEDAGVNDVIAAQVDKIESKKIYFNTFDEKKQSIRLNKDVVYFDNEGKDLGEVDTDDYVVLVDTDNDSDSIDYVLYVISEDDAKTNGYLKKDDNKETLMDQFMKQNWKDDKPDPGEDDSDIDFTSSNYEKYPGETYLYAVAGTLKGEAKDADEVKVEIGSESKTVTVKDGAKFSAGLFVDTLEKTATVTAYKDGEKLYSTEVKLSAK

SEQ ID NO: 2 - protein, 163 aa, Brevibacillus laterosporus
DPAMEKVVKRLSALGLAAGDDKGNFNLDQTITRSEFATLMVRARGLEEAAKTATFNPFFKDVIATDWFQGFVSIAVSQDLAKGYPDKTFKPKNSVTYAEAATMLVRALGYEPLALQKGTWPNNFISQASELGISQGVTIDANKPAVRGDVFKMLDNSLRIDLM

SEQ ID NO: 3 - protein, 34 aa, Brevibacillus laterosporus
AMEKVVKRLSALGLAAGDDKGNFNLDQTITRSEF

SEQ ID NO: 4 - protein, 42 aa, Brevibacillus laterosporus
FKDVIATDWFQGFVSIAVSQDLAKGYPDKTFKPKNSVTYAEA

SEQ ID NO: 5 - protein, 152 aa, Brevibacillus laterosporus
WPNNFISQASELGISQGVTIDANKPAVRGDVFKMLDNSLRIDLMKQTTVGSQNNHEVLKGKNLLNEYLKVQVYDTKWTMTDRNKPEDLPSVSNVPVIGLGSLKYNEVELTSDSRSFFNKVKYKVADSINANDYAGQRVQVWIKDRDNTVVWM

SEQ ID NO: 6 - protein, 73 aa, Brevibacillus laterosporus
LMKQTTVGSQNNHEVLKGKNLLNEYLKVQVYDTKWTMTDRNKPEDLPSVSNVPVIGLGSLKYNEVELTSDSRS

SEQ ID NO: 7 - protein, 257 aa, Brevibacillus laterosporus
QTTVGSQNNHEVLKGKNLLNEYLKVQVYDTKWTMTDRNKPEDLPSVSNVPVIGLGSLKYNEVELTSDSRSFFNKVKYKVADSINANDYAGQRVQVWIKDRDNTVVWMEGSEDETVVFDKYSALHISGKKLSPTKDKDQIKKDKFNKLTLELDSGTKYDFAEDVSVTFNFERATKASDVQEMLQKVLEADDAYSVKVVLNTKNEIRYISIVDDRSGDQNWVNNHFGSELIKKVTDKKITTLVGKDLALEDKKEGIDYL

SEQ ID NO: 8 - protein, 858 aa, Brevibacillus laterosporus
MATSAFAASTGYYLGGNIEKYYGAQALINANVASAFQNEVFKSSLTQSQVLYVNNQGKVASLQDIVDAIDNGQTSTEAFREATGADFDKIGGEEGFHTVLEDGTVDPEKKKVPEQDEQPAGDLKVESVSAINASQIKIVFSQKVDETSAESPANVSFDGFTATGYELKDDEKTLIVTTTTPITKKKDVVVTVKNVTLAGDNTVVVPTFTKVITVEDTTPADVTKVESKTSGATATSVDIYFSEPVQSGIVKINGQQYAASFTGNPEKVTVSNLDLDATKTHTLEIVNLTDTAGNKSTVTKSFTITADATAPQVATLSQYSDKAVLVTFDKKMEATTITAGSITIADESLNPVDAGAIGQLPGDTTGTKFIVPINSTLYGTKSSVTLNLAFKDTITDKLGNKIVPSTKQVTLTKDVAAPAVDKVTVKKDAAGKVESLVLDFNKPLKAGTYASDKITVINQDGVDVTGDLLADGTNVAFEADSEAVAAGAKKVEFKLKVATALTGEFTFTFAKGLVQDDAETANDSVSYAKSINFGNADAPEFEITESDVTEAGTNKIQVAFGKTVKGGNVAGSATAVSNYTLNGSALPAGTLITLNTNKDTATITLPASSIPTSDTHAVFTISNVQTLTGLTIKPLTATVAIVDNVAPVLESAKVLDDVTIELTYSEKISLNDTDVFDSFKIVNGTTAIPFTTGELVAGSVSGFDSKIKISINKPGTSDIPAVPPTPGTATVGGKDAGIANIDDQTTANADGTVTYTVAVNGSELQVSDGTKVVTTLDASGDGSFKVNGVTVTITGGADTNTFTVTTEAAVDGTPLVPGTPATKLDLTKAITVETKTTTAVKDVAGNAHKASVLVNVSK

SEQ ID NO: 9 - protein, 211 aa, Brevibacillus laterosporus
NIEKYYGAQALINANVASAFQNEVFKSSLTQSQVLYVNNQGKVASLQDIVDAIDNGQTSTEAFREATGADFDKIGGEEGFHTVLEDGTVDPEKKKVPEQDEQPAGDLKVESVSAINASQIKIVFSQKVDETSAESPANVSFDGFTATGYELKDDEKTLIVTTTTPITKKKDVVVTVKNVTLAGDNTVVVPTFTKVITVEDTTPADVTKVES

SEQ ID NO: 10 - protein, 162 aa, Brevibacillus laterosporus
QPAGDLKVESVSAINASQIKIVFSQKVDETSAESPANVSFDGFTATGYELKDDEKTLIVTTTTPITKKKDVVVTVKNVTLAGDNTVVVPTFTKVITVEDTTPADVTKVESKTSGATATSVDIYFSEPVQSGIVKINGQQYAASFTGNPEKVTVSNLDLDATK

SEQ ID NO: 11 - protein, 93 aa, Brevibacillus laterosporus
SVSAINASQIKIVFSQKVDETSAESPANVSFDGFTATGYELKDDEKTLIVTTTTPITKKKDVVVTVKNVTLAGDNTVVVPTFTKVITVEDTTP

SEQ ID NO: 12 - protein, 171 aa, Brevibacillus laterosporus
IVKINGQQYAASFTGNPEKVTVSNLDLDATKTHTLEIVNLTDTAGNKSTVTKSFTITADATAPQVATLSQYSDKAVLVTFDKKMEATTITAGSITIADESLNPVDAGAIGQLPGDTTGTKFIVPINSTLYGTKSSVTLNLAFKDTITDKLGNKIVPSTKQVTLTKDVAAPA

SEQ ID NO: 13 - protein, 294 aa, Brevibacillus laterosporus
LPGDTTGTKFIVPINSTLYGTKSSVTLNLAFKDTITDKLGNKIVPSTKQVTLTKDVAAPAVDKVTVKKDAAGKVESLVLDFNKPLKAGTYASDKITVINQDGVDVTGDLLADGTNVAFEADSEAVAAGAKKVEFKLKVATALTGEFTFTFAKGLVQDDAETANDSVSYAKSINFGNADAPEFEITESDVTEAGTNKIQVAFGKTVKGGNVAGSATAVSNYTLNGSALPAGTLITLNTNKDTATITLPASSIPTSDTHAVFTISNVQTLTGLTIKPLTATVAIVDNVAPVLESAK

SEQ ID NO: 14 - protein, 80 aa, Brevibacillus laterosporus
KVESLVLDFNKPLKAGTYASDKITVINQDGVDVTGDLLADGTNVAFEADSEAVAAGAKKVEFKLKVATALTGEFTFTFAK

SEQ ID NO: 15 - protein, 156 aa, Brevibacillus laterosporus
SEAVAAGAKKVEFKLKVATALTGEFTFTFAKGLVQDDAETANDSVSYAKSINFGNADAPEFEITESDVTEAGTNKIQVAFGKTVKGGNVAGSATAVSNYTLNGSALPAGTLITLNTNKDTATITLPASSIPTSDTHAVFTISNVQTLTGLTIKPLT

SEQ ID NO: 16 - protein, 160 aa, Brevibacillus laterosporus
EFEITESDVTEAGTNKIQVAFGKTVKGGNVAGSATAVSNYTLNGSALPAGTLITLNTNKDTATITLPASSIPTSDTHAVFTISNVQTLTGLTIKPLTATVAIVDNVAPVLESAKVLDDVTIELTYSEKISLNDTDVFDSFKIVNGTTAIPFTTGELVAGS

SEQ ID NO: 17 - protein, 254 aa, Brevibacillus laterosporus
AFGKTVKGGNVAGSATAVSNYTLNGSALPAGTLITLNTNKDTATITLPASSIPTSDTHAVFTISNVQTLTGLTIKPLTATVAIVDNVAPVLESAKVLDDVTIELTYSEKISLNDTDVFDSFKIVNGTTAIPFTTGELVAGSVSGFDSKIKISINKPGTSDIPAVPPTPGTATVGGKDAGIANIDDQTTANADGTVTYTVAVNGSELQVSDGTKVVTTLDASGDGSFKVNGVTVTITGGADTNTFTVTTEAAVDG

SEQ ID NO: 18 - protein, 73 aa, Brevibacillus laterosporus
NKDTATITLPASSIPTSDTHAVFTISNVQTLTGLTIKPLTATVAIVDNVAPVLESAKVLDDVTIELTYSEKIS

SEQ ID NO: 19 - protein, 127 aa, Brevibacillus laterosporus
DTDVFDSFKIVNGTTAIPFTTGELVAGSVSGFDSKIKISINKPGTSDIPAVPPTPGTATVGGKDAGIANIDDQTTANADGTVTYTVAVNGSELQVSDGTKVVTTLDASGDGSFKVNGVTVTITGGAD

SEQ ID NO: 20 - protein, 111 aa, Brevibacillus laterosporus
PTPGTATVGGKDAGIANIDDQTTANADGTVTYTVAVNGSELQVSDGTKVVTTLDASGDGSFKVNGVTVTITGGADTNTFTVTTEAAVDGTPLVPGTPATKLDLTKAITVET

SEQ ID NO: 21 - protein, 79 aa, Brevibacillus laterosporus
GKDAGIANIDDQTTANADGTVTYTVAVNGSELQVSDGTKVVTTLDASGDGSFKVNGVTVTITGGADTNTFTVTTEAAVD

SEQ ID NO: 22 - protein, 398 aa, Brevibacillus laterosporus
MVVEKECTNMNFPYSKKWVALTVAAMVMTSGSGLAMAAATTPAKVTTQEVNVKKEQEVGANDLTVEKAVELTLENNIDYKKAKIDVDNALIRVNNANYSGAKIKEDMIENLGTAQGKYVATVRAEGQKEIGKLSLKRSEGSFKLGTEKVYFDLLNAEDDLVLKKQSLERSESQLKIANAAFSVGTRAKTDVLQAEAGVAQAKAAVAQAESATDIARLSLNKFMGVDLNKKWNLLTKQTSEKVEVMDVEKAIEKALKERVDILTAEQEIKNSEKELEVIEKYSILSGFTGQEAQNTYEKNKLTLTQTQNQVKMDLMQSYYNLNSIKTSLEALKKGTEAASESYRLTKLRFENGLATAIEVVSAEEELAKAENNYQMNLYKLNLAVMAYENAILQPASGQ

SEQ ID NO: 23 - protein, 372 aa, Brevibacillus laterosporus
ALTVAAMVMTSGSGLAMAAATTPAKVTTQEVNVKKEQEVGANDLTVEKAVELTLENNIDYKKAKIDVDNALIRVNNANYSGAKIKEDMIENLGTAQGKYVATVRAEGQKEIGKLSLKRSEGSFKLGTEKVYFDLLNAEDDLVLKKQSLERSESQLKIANAAFSVGTRAKTDVLQAEAGVAQAKAAVAQAESATDIARLSLNKFMGVDLNKKWNLLTKQTSEKVEVMDVEKAIEKALKERVDILTAEQEIKNSEKELEVIEKYSILSGFTGQEAQNTYEKNKLTLTQTQNQVKMDLMQSYYNLNSIKTSLEALKKGTEAASESYRLTKLRFENGLATAIEVVSAEEELAKAENNYQMNLYKLNLAVMAYENAI

SEQ ID NO: 24 - protein, 360 aa, Brevibacillus laterosporus
SGLAMAAATTPAKVTTQEVNVKKEQEVGANDLTVEKAVELTLENNIDYKKAKIDVDNALIRVNNANYSGAKIKEDMIENLGTAQGKYVATVRAEGQKEIGKLSLKRSEGSFKLGTEKVYFDLLNAEDDLVLKKQSLERSESQLKIANAAFSVGTRAKTDVLQAEAGVAQAKAAVAQAESATDIARLSLNKFMGVDLNKKWNLLTKQTSEKVEVMDVEKAIEKALKERVDILTAEQEIKNSEKELEVIEKYSILSGFTGQEAQNTYEKNKLTLTQTQNQVKMDLMQSYYNLNSIKTSLEALKKGTEAASESYRLTKLRFENGLATAIEVVSAEEELAKAENNYQMNLYKLNLAVMAYENAI

SEQ ID NO: 25 - protein, 335 aa, Brevibacillus laterosporus
ANDLTVEKAVELTLENNIDYKKAKIDVDNALIRVNNANYSGAKIKEDMIENLGTAQGKYVATVRAEGQKEIGKLSLKRSEGSFKLGTEKVYFDLLNAEDDLVLKKQSLERSESQLKIANAAFSVGTRAKTDVLQAEAGVAQAKAAVAQAESATDIARLSLNKFMGVDLNKKWNLLTKQTSEKVEVMDVEKAIEKALKERVDILTAEQEIKNSEKELEVIEKYSILSGFTGQEAQNTYEKNKLTLTQTQNQVKMDLMQSYYNLNSIKTSLEALKKGTEAASESYRLTKLRFENGLATAIEVVSAEEELAKAENNYQMNLYKLNLAVMAYENAILQP

SEQ ID NO: 26 - protein, 332 aa, Brevibacillus laterosporus
LTVEKAVELTLENNIDYKKAKIDVDNALIRVNNANYSGAKIKEDMIENLGTAQGKYVATVRAEGQKEIGKLSLKRSEGSFKLGTEKVYFDLLNAEDDLVLKKQSLERSESQLKIANAAFSVGTRAKTDVLQAEAGVAQAKAAVAQAESATDIARLSLNKFMGVDLNKKWNLLTKQTSEKVEVMDVEKAIEKALKERVDILTAEQEIKNSEKELEVIEKYSILSGFTGQEAQNTYEKNKLTLTQTQNQVKMDLMQSYYNLNSIKTSLEALKKGTEAASESYRLTKLRFENGLATAIEVVSAEEELAKAENNYQMNLYKLNLAVMAYENAILQP

SEQ ID NO: 27 - protein, 318 aa, Brevibacillus laterosporus
LTVEKAVELTLENNIDYKKAKIDVDNALIRVNNANYSGAKIKEDMIENLGTAQGKYVATVRAEGQKEIGKLSLKRSEGSFKLGTEKVYFDLLNAEDDLVLKKQSLERSESQLKIANAAFSVGTRAKTDVLQAEAGVAQAKAAVAQAESATDIARLSLNKFMGVDLNKKWNLLTKQTSEKVEVMDVEKAIEKALKERVDILTAEQEIKNSEKELEVIEKYSILSGFTGQEAQNTYEKNKLTLTQTQNQVKMDLMQSYYNLNSIKTSLEALKKGTEAASESYRLTKLRFENGLATAIEVVSAEEELAKAENNYQMNLYKL

SEQ ID NO: 28 - protein, 121 aa, Brevibacillus laterosporus
LGTEKVYFDLLNAEDDLVLKKQSLERSESQLKIANAAFSVGTRAKTDVLQAEAGVAQAKAAVAQAESATDIARLSLNKFMGVDLNKKWNLLTKQTSEKVEVMDVEKAIEKALKERVDILTA

SEQ ID NO: 29 - protein, 156 aa, Brevibacillus laterosporus
KQTSEKVEVMDVEKAIEKALKERVDILTAEQEIKNSEKELEVIEKYSILSGFTGQEAQNTYEKNKLTLTQTQNQVKMDLMQSYYNLNSIKTSLEALKKGTEAASESYRLTKLRFENGLATAIEVVSAEEELAKAENNYQMNLYKLNLAVMAYENAI

SEQ ID NO: 30 - protein, 102 aa, Brevibacillus laterosporus
QEAQNTYEKNKLTLTQTQNQVKMDLMQSYYNLNSIKTSLEALKKGTEAASESYRLTKLRFENGLATAIEVVSAEEELAKAENNYQMNLYKLNLAVMAYENAI

SEQ ID NO: 31 - protein, 78 aa, Brevibacillus laterosporus
AQNTYEKNKLTLTQTQNQVKMDLMQSYYNLNSIKTSLEALKKGTEAASESYRLTKLRFENGLATAIEVVSAEEELAKA

SEQ ID NO: 32 - protein, 1083 aa, Brevibacillus laterosporus
MQDSGWKKKETTSNKISQKQLLYIRGGEPKVMKKVVNSLLASALALSVVPVVAGAEEVNTKGTEETSTTAPQLDPAMEKVVKRLSALGLAAGDDKGNFNLDQTITRSEFATLMVRARGLEEAAKTATFNPFFKDVIATDWFQGFVSIAVSQDLAKGYPDKTFKPKNSVTYAEAATMLVRALGYEPLALQKGTWPNNFISQASELGISQGVTIDANKPAVRGDVFKMLDNSLRIDLMKQTTVGSQNNHEVLKGKNLLNEYLKVQVYDTKWTMTDRNKPEDLPSVSNVPVIGLGSLKYNEVELTSDSRSFFNKVKYKVADSINANDYAGQRVQVWIKDRDNTVVWMEGSEDETVVFDKYSALHISGKKLSPTKDKDQIKKDKFNKLTLELDSGTKYDFAEDVSVTFNFERATKASDVQEMLQKVLEADDAYSVKVVLNTKNEIRYISIVDDRSGDQNWVNNHFGSELIKKVTDKKITTLVGKDLALEDKKEGIDYLVFLDGKPAKLADLKEMDVYHAYRADGNKDKLLIFANRSTKEGKVEKVRSKSESDYRIVIDGKEYRFRNASFAKDKDKEIEDQITPDDIDRLEGTKVKVYLDAVGRIRHIEGYEANDRKQMAIISRSGTYDPYSDQYTFKVYTEKGNSKTVKLDGKYVKNEEGEKFSDLKKNESEKKIAEKYLTLSKDKNFILAQIELDASGDIEKVELRDTSKAKHMSKDQWKKDSDKDDETVKFGGKTYDFSSSSVVFDGSKGIQSGDVTSLKSPGTIKWSKIAEKEAEVYFTVDGSDVEAVFVVDSKGIASGSEYGVVVEAYRSSGVDKLKIKHRDDDGKWVEKEYKYDGDGKDKLEKTFIRFSADSDDLIDEDDVRVIVDARSDDEYKYKILKEKDYEDAGVNEVIPAQVDKIESKKIYFNTFDEKKQSIRLNKDVVYFDNEGKDLGEVDTDDYVVLVDTDNDSDSIDYVLYVISEDDAKTNGYLKKDDNKETLMDQFMKQNWKDDKPDPGDDGDLDITKQTASEVLPGVYGYNIVGKLTGDLKDEEEVTIVLGDREFTVEVTDGKFEFTKSYKKVDEAYVKVGKEKFDLNIKFDK

SEQ ID NO: 33 - protein, 163 aa, Brevibacillus laterosporus; identical to SEQ ID NO: 2

SEQ ID NO: 34 - protein, 34 aa, Brevibacillus laterosporus; identical to SEQ ID NO: 3

SEQ ID NO: 35 - protein, 42 aa, Brevibacillus laterosporus; identical to SEQ ID NO: 4

SEQ ID NO: 36 - protein, 80 aa, Brevibacillus laterosporus
LAKGYPDKTFKPKNSVTYAEAATMLVRALGYEPLALQKGTWPNNFISQASELGISQGVTIDANKPAVRGDVFKMLDNSLR

SEQ ID NO: 37 - protein, 73 aa, Brevibacillus laterosporus; identical to SEQ ID NO: 6

SEQ ID NO: 38 - protein, 1133 aa, Brevibacillus laterosporus
MKFNKAVDSIDAKNFTIAGATVEVASLSEDKKTATLTVSGLNYDTEYTIVAKDVLVDGKPITLKESKFKTPVITDLYKLELTTDAAGDQLKSDGVDTLVITAKLLDKVTGLVDENADNVVISFSTTYGNLANTRVTVQKGIATVTLRSEFSQNDLVAKVDAQVIEASGDYKYLIGKVIGTKEVYFKVNPGDTGEIDQLPVVQSAGSNQADRVTVYFNKNVDVADFVQKDETTGKYKVDANKNALLKTGTEIKVTQDNVAKKVRGLKKVEGNPKALEVILDKTEKVGDTWTNILTDNKPVKVEFVQTNTAGPQTTKAEFILADARKPEVTSVDANGLKTVVVKFSESIAQAQVSLDGGLTVIKELEFGEFDQATLEDKRDQLTIRTLDYLKAGTHSVQLSSIYDFAGLTDDKNISTSQTLDFEVKADNAIPSSTVSVESPEQFRVTFNKEVDGLTADKVKLQVAVKNADGTESWVNVADAAGKYKVTPELAMDDTAENKNEYVFELKTDWTEIYDTKVTTKNYYNDKYRLFIPASSVTNPANGKHNADIEMLLNDTIMTTPDTVSPTIKDITQISTGRYNVEMSKPVKLPGKDLDTPSQSQPKVPEPIIEFLGKDKDGNTKTIKGKVVDYTDKNRADKQFEVVPTPENLEDLPQTIVNNGGDIHWTLVVRSISDDVGNTAETLTKVFEIKPDAVVADEVFMVKGKLSDNTLYNGVVGAENGDGPDTITLTFTSGVQYTGSEKNAVNPANYTLDGENLPKGTILTVSDSDDKPENGYDTVIISLPDGSLKTSKGSNLITVSKSLESYKGAKLTGEYAITFVPEAGITPDEAKVADAVAKAITNLPSVDKLTLSDEATLVDVRTQYNALNAIQQKAVKNLKVLEAAEKKMAELKTAAGDVAANLAAAKTAVTDLETAAGKDLTKEADLLAAEAALKTAKEKVALVADANEKKLLDAKVTLAEKTVTDARAKFDGDAATALKDAKAAVEALEDAAKEDLTVEAKLTAAETAVADAEATVAKVAAGAEKTELEGKVAAAKKTVTDARTEFDKAKEAAELKAAKDALEEAITAAKEQNSVAVAGTNAGEYPQVAKDAYNDEIQEAEVVFNNAAATKAELEAAKVALDTAKATFEGTVIS

SEQ ID NO: 39 - protein, 124 aa, Brevibacillus laterosporus
LETAAGKDLTKEADLLAAEAALKTAKEKVALVADANEKKLLDAKVTLAEKTVTDARAKFDGDAATALKDAKAAVEALEDAAKEDLTVEAKLTAAETAVADAEATVAKVAAGAEKTELEGKVAAA

SEQ ID NO: 40 - protein, 92 aa, Brevibacillus laterosporus
KTAKEKVALVADANEKKLLDAKVTLAEKTVTDARAKFDGDAATALKDAKAAVEALEDAAKEDLTVEAKLTAAETAVADAEATVAKVAAGAEK

SEQ ID NO: 41 - protein, 189 aa, Brevibacillus laterosporus
ADANEKKLLDAKVTLAEKTVTDARAKFDGDAATALKDAKAAVEALEDAAKEDLTVEAKLTAAETAVADAEATVAKVAAGAEKTELEGKVAAAKKTVTDARTEFDKAKEAAELKAAKDALEEAITAAKEQNSVAVAGTNAGEYPQVAKDAYNDEIQEAEVVFNNAAATKAELEAAKVALDTAKATFEGTV

SEQ ID NO: 42 - protein, 101 aa, Brevibacillus laterosporus
DANEKKLLDAKVTLAEKTVTDARAKFDGDAATALKDAKAAVEALEDAAKEDLTVEAKLTAAETAVADAEATVAKVAAGAEKTELEGKVAAAKKTVTDARTE

SEQ ID NO: 43 - protein, 121 aa, Brevibacillus laterosporus
VTLAEKTVTDARAKFDGDAATALKDAKAAVEALEDAAKEDLTVEAKLTAAETAVADAEATVAKVAAGAEKTELEGKVAAAKKTVTDARTEFDKAKEAAELKAAKDALEEAITAAKEQNSVA

SEQ ID NO: 44 - protein, 79 aa, Brevibacillus laterosporus
DAATALKDAKAAVEALEDAAKEDLTVEAKLTAAETAVADAEATVAKVAAGAEKTELEGKVAAAKKTVTDARTEFDKAKE

SEQ ID NO: 45 - protein, 144 aa, Brevibacillus laterosporus
AAVEALEDAAKEDLTVEAKLTAAETAVADAEATVAKVAAGAEKTELEGKVAAAKKTVTDARTEFDKAKEAAELKAAKDALEEAITAAKEQNSVAVAGTNAGEYPQVAKDAYNDEIQEAEVVFNNAAATKAELEAAKVALDTAKA

SEQ ID NO: 46 - protein, 90 aa, Brevibacillus laterosporus
KKTVTDARTEFDKAKEAAELKAAKDALEEAITAAKEQNSVAVAGTNAGEYPQVAKDAYNDEIQEAEVVFNNAAATKAELEAAKVALDTAK

SEQ ID NO: 47 - protein, 84 aa, Brevibacillus laterosporus
KAKEAAELKAAKDALEEAITAAKEQNSVAVAGTNAGEYPQVAKDAYNDEIQEAEVVFNNAAATKAELEAAKVALDTAKATFEGT

SEQ ID NO: 48 - protein, 389 aa, Brevibacillus laterosporus
MNFPYSKKWVALTVAAMVMTSGSGLAMAATTTPAKVTTQEVNVKKEQEVGANDLTVEKAVELTLENNIDYKKAKIDVDNALIRVNNANYSGAKIKEDMIENLGTAQGKYVATVRAEGQKEIGKLSLKRSEGSFKLGTEKVYFDLLNAEDDLVLKKQSLERSESQLKIANAAFSVGTRAKTDVLQAEAGVAQAKAAVAQAESATDIARLSLNKFMGVDLNKKWNLLTKQTSEKVEVMDVEKAIEKALKERVDILTAEQEIKNSEKELEVIEKYSILSGFTGQEAQNTYEKNKLTLTQTQNQVKMDLMQSYYNLNSIKTSLEALKKGTEAASESYRLTKLRFENGLATAIEVVSAEEELAKAENNYQMNLYKLNLAVMAYENAILQPASGQ

SEQ ID NO: 49 - protein, 360 aa, Brevibacillus laterosporus
SGLAMAATTTPAKVTTQEVNVKKEQEVGANDLTVEKAVELTLENNIDYKKAKIDVDNALIRVNNANYSGAKIKEDMIENLGTAQGKYVATVRAEGQKEIGKLSLKRSEGSFKLGTEKVYFDLLNAEDDLVLKKQSLERSESQLKIANAAFSVGTRAKTDVLQAEAGVAQAKAAVAQAESATDIARLSLNKFMGVDLNKKWNLLTKQTSEKVEVMDVEKAIEKALKERVDILTAEQEIKNSEKELEVIEKYSILSGFTGQEAQNTYEKNKLTLTQTQNQVKMDLMQSYYNLNSIKTSLEALKKGTEAASESYRLTKLRFENGLATAIEVVSAEEELAKAENNYQMNLYKLNLAVMAYENAI

SEQ ID NO: 50 - protein, 331 aa, Brevibacillus laterosporus
NDLTVEKAVELTLENNIDYKKAKIDVDNALIRVNNANYSGAKIKEDMIENLGTAQGKYVATVRAEGQKEIGKLSLKRSEGSFKLGTEKVYFDLLNAEDDLVLKKQSLERSESQLKIANAAFSVGTRAKTDVLQAEAGVAQAKAAVAQAESATDIARLSLNKFMGVDLNKKWNLLTKQTSEKVEVMDVEKAIEKALKERVDILTAEQEIKNSEKELEVIEKYSILSGFTGQEAQNTYEKNKLTLTQTQNQVKMDLMQSYYNLNSIKTSLEALKKGTEAASESYRLTKLRFENGLATAIEVVSAEEELAKAENNYQMNLYKLNLAVMAYENAI

SEQ ID NO: 51 - protein, 334 aa, Brevibacillus laterosporus
NDLTVEKAVELTLENNIDYKKAKIDVDNALIRVNNANYSGAKIKEDMIENLGTAQGKYVATVRAEGQKEIGKLSLKRSEGSFKLGTEKVYFDLLNAEDDLVLKKQSLERSESQLKIANAAFSVGTRAKTDVLQAEAGVAQAKAAVAQAESATDIARLSLNKFMGVDLNKKWNLLTKQTSEKVEVMDVEKAIEKALKERVDILTAEQEIKNSEKELEVIEKYSILSGFTGQEAQNTYEKNKLTLTQTQNQVKMDLMQSYYNLNSIKTSLEALKKGTEAASESYRLTKLRFENGLATAIEVVSAEEELAKAENNYQMNLYKLNLAVMAYENAILQP

SEQ ID NO: 52 - protein, 318 aa, Brevibacillus laterosporus; identical to SEQ ID NO: 27

SEQ ID NO: 53 - protein, 332 aa, Brevibacillus laterosporus; identical to SEQ ID NO: 26

SEQ ID NO: 54 - protein, 121 aa, Brevibacillus laterosporus; identical to SEQ ID NO: 28

SEQ ID NO: 55 - protein, 156 aa, Brevibacillus laterosporus; identical to SEQ ID NO: 29

SEQ ID NO: 56 - protein, 102 aa, Brevibacillus laterosporus; identical to SEQ ID NO: 30

SEQ ID NO: 57 - protein, 78 aa, Brevibacillus laterosporus; identical to SEQ ID NO: 31

SEQ ID NO: 58 - protein, 1099 aa, Brevibacillus laterosporus
MLFFLICDPAKGGETRLQDSGWKKKETTSNKISQNQLLYIRGGEPKVMKKVVNSLLASALALSVVPVVAGAEEVKNTGTEETTPAPQLDPAMEKVVKRLSALGLAAGDDKGNFNLDQTITRSEFATLMVRARGLEEAAKTATFNPFFKDVIATDWFQGFVSIAVSQDLAKGYPDKTFKPKNSVTYAEAATMLVRALGYEPLALQKGTWPNNFISQASELGISQGVTIDANKPAVRGDVFKMLDNSLRIDLMKQTTVGSQNNHEVLKGKNLLNEYLKVQVYDTKWTMTDRNKPEDLPSVSNVPVIGLGSLKYNEVELTSDSRSFFNKVKYKVADSINANDYAGQRVQVWIKDRDNTVVWMEGSEDETVVFDKYSALHISGKKLSPTKDKDQIKKDKFNKLTLELDSGTKYDFAEDVSVTFNFERATKASDVQEMLQKVLEADDAYSVKVVLNTKNEIRYISIVDDRSGDQNWVNNHFGSELIKKVTDKKITTLVGKDLALEDKKEGIDYLVFLDGKPAKLADLKEMDVYHAYRADGNKDKLLIFANRSTKEGKVEKVRSKSESDYRIVIDGKEYRFRNASFAKDKDKEIEDQITPDDIDRLEGTKVKVYLDAVGRIRHIEGYEANDRKQMAIISRSGTYDPYSDQYTFKVYTEKGNSKTVKLDGKYVKNEEGEKFSDLKKNESEKKIAEKYLTLSKDKNFILAQIELDASGDIEKVELKDTSKAKHMSKDQWKKDSDKDDETVKFGGKTYDFSSSSVVFDGSKGIQSGDVTSLKSPGTIKWSKIAEKEAEVYFTVDGSDVEAVFVVDSKGIASGSEYGVVVEAYRSSGVDKLKIKHRDDDGKWVEKEYKYDGDGKDKLEKTFIRFSADSDDLIDEDDVRVIVDARSDDEYKYKILKEKDYEDAGVNEVIPAQVDKIESKKIYFNTFDEKKQSIRLNKDVVYFDNEGKDLGEVDTDDYVVLVDTDNDSDSIDYVLYVISEDDAKTNGYLKKDDNKETLMDQFMKQNWKDDKPDPGEDDGDLDITKQTASEVLPGVYGYNIVGKLTGDLKDEEEVTIVLGDREFTVEVTDGKFEFTKSYKKVDEAYVKVGKEKFDLNIKFDK

SEQ ID NO: 59 - protein, 163 aa, Brevibacillus laterosporus; identical to SEQ ID NO: 2

SEQ ID NO: 60 - protein, 34 aa, Brevibacillus laterosporus; identical to SEQ ID NO: 3

SEQ ID NO: 61 - protein, 42 aa, Brevibacillus laterosporus; identical to SEQ ID NO: 4

SEQ ID NO: 62 - protein, 152 aa, Brevibacillus laterosporus; identical to SEQ ID NO: 5

SEQ ID NO: 63 - protein, 73 aa, Brevibacillus laterosporus; identical to SEQ ID NO: 6

SEQ ID NO: 64 - protein, 1285 aa, Brevibacillus laterosporus
MNKKVILSVLSTALVTSMATSAFAASGGVYIGGNVDRHYSDEALLKQHAMLVADLYDSGLENVEDNVLFVNWDGEVATLQQLVDAKNAGKEVEYKTVTSEDFEKIGSEEGFYAVDADGNVSTEKELQPEQKPVAPGDLNVESVSAITKTKVEVKFNKAVDSIDAKNFTIAGATVEVASLSEDKKTATLTVSGLNYDTEYTIVAKDVLVDGKPITLKESKFKTPVITDLYKLELTTDAAGDQLKSDGVDTLVITAKLLDKVTGLVDENADNVVISFSTTYGNLANTRVTVQKGIATVTLRSEFSQNDLVAKVDAQVIEASGDYKYLIGKVIGTKEVYFKVNPGDTGEIDQLPVVQSAGSNQADRVTVYFNKNVDVADFVQKDETTGKYKVDANKNALLKTGTEIKVTQDNVAKKVRGLKKVEGNPKALEVILDKTEKVGDTWTNILTDNKPVKVEFVQTNTAGPQTTKAEFILADARKPEVTSVDANGLKTVVVKFSESIAQAQVSLDGGLTVIKELEFGEFDQATLEDKRDQLTIRTLDYLKAGTHSVQLSSIYDFAGLTDDKNISTSQTLDFEVKADNAIPSSTVSVESPEQFRVTFNKEVDGLTADKVKLQVAVKNADGTESWVNVADAAGKYKVTPELAMDDTAENKNEYVFELKTDWTEIYDTKVTTKNYYNDKYRLFIPASSVTNPANGKHNADIEMLLNDTIMTTPDTVSPTIKDITQISTGRYNVEMSKPVKLPGKDLDTPSQSQPKVPEPIIEFLGKDKDGNTKTIKGKVVDYTDKNRADKQFEVVPTPENLEDLPQTIVNNGGDIHWTLVVRSISDDVGNTAETLTKVFEIKPDAVVADEVFMVKGKLSDNTLYNGVVGAENGDGPDTITLTFTSGVQYTGSEKNAVNPANYTLDGENLPKGTILTVSDSDDKPENGYDTVIISLPDGSLKTSKGSNLITVSKSLESYKGAKLTGEYAITFVPEAGITPDEAKVADAVAKAITNLPSVDKLTLSDEATLVDVRTQYNALNAIQQKAVKNLKVLEAAEKKMAELKTAAGDVAANLAAAKTAVTDLETAAGKDLTKEADLLAAEAALKTAKEKVALVADANEKKLLDAKVTLAEKTVTDARAKFDGDAATALKDAKAAVEALEDAAKEDLTVEAKLTAAETAVADAEATVAKVAAGAEKTELEGKVAAAKKTVTDARTEFDKAKEAAELKAAKDALEEAITAAKEQNSVAVAGTNAGEYPQVAKDAYNDEIQEAEVVFNNAAATKAELEAAKVALDTAKATFEGTVIS

SEQ ID NO: 65 - protein, 101 aa, Brevibacillus laterosporus
GDLNVESVSAITKTKVEVKFNKAVDSIDAKNFTIAGATVEVASLSEDKKTATLTVSGLNYDTEYTIVAKDVLVDGKPITLKESKFKTPVITDLYKLELTTD

SEQ ID NO: 66 - protein, 43 aa, Brevibacillus laterosporus
LPVVQSAGSNQADRVTVYFNKNVDVADFVQKDETTGKYKVDAN

SEQ ID NO: 67 - protein, 74 aa, Brevibacillus laterosporus
STVSVESPEQFRVTFNKEVDGLTADKVKLQVAVKNADGTESWVNVADAAGKYKVTPELAMDDTAENKNEYVFEL

SEQ ID NO: 68 - protein, 124 aa, Brevibacillus laterosporus; identical to SEQ ID NO: 39

SEQ ID NO: 69 - protein, 92 aa, Brevibacillus laterosporus; identical to SEQ ID NO: 40

SEQ ID NO: 70 - protein, 189 aa, Brevibacillus laterosporus; identical to SEQ ID NO: 41

SEQ ID NO: 71 - protein, 101 aa, Brevibacillus laterosporus; identical to SEQ ID NO: 42

SEQ ID NO: 72 - protein, 121 aa, Brevibacillus laterosporus; identical to SEQ ID NO: 43

SEQ ID NO: 73 - protein, 80 aa, Brevibacillus laterosporus
GDAATALKDAKAAVEALEDAAKEDLTVEAKLTAAETAVADAEATVAKVAAGAEKTELEGKVAAAKKTVTDARTEFDKAKE

SEQ ID NO: 74 - protein, 144 aa, Brevibacillus laterosporus; identical to SEQ ID NO: 45

SEQ ID NO: 75 - protein, 90 aa, Brevibacillus laterosporus; identical to SEQ ID NO: 46

SEQ ID NO: 76 - protein, 77 aa, Brevibacillus laterosporus
ELKAAKDALEEAITAAKEQNSVAVAGTNAGEYPQVAKDAYNDEIQEAEVVFNNAAATKAELEAAKVALDTAKATFEG

SEQ ID NO: 77 - protein, 389 aa, Brevibacillus laterosporus; identical to SEQ ID NO: 48

SEQ ID NO: 78 - protein, 360 aa, Brevibacillus laterosporus; identical to SEQ ID NO: 49

SEQ ID NO: 79 - protein, 331 aa, Brevibacillus laterosporus; identical to SEQ ID NO: 50

SEQ ID NO: 80 - protein, 334 aa, Brevibacillus laterosporus; identical to SEQ ID NO: 51

SEQ ID NO: 81 - protein, 318 aa, Brevibacillus laterosporus; identical to SEQ ID NO: 27

SEQ ID NO: 82 - protein, 332 aa, Brevibacillus laterosporus; identical to SEQ ID NO: 26

SEQ ID NO: 83 - protein, 121 aa, Brevibacillus laterosporus; identical to SEQ ID NO: 28

SEQ ID NO: 84 - protein, 156 aa, Brevibacillus laterosporus; identical to SEQ ID NO: 29

SEQ ID NO: 85 - protein, 102 aa, Brevibacillus laterosporus; identical to SEQ ID NO: 30

SEQ ID NO: 86 - protein, 78 aa, Brevibacillus laterosporus; identical to SEQ ID NO: 31

SEQ ID NO: 87 - protein, 1090 aa, Brevibacillus laterosporus
MQDSGWKKKETTSNKISQNQLLYIRGGEPKVMKKVVNSLLASALALSVVPVVAGAEEVKNTGTEETSTTAPQLDPAMEKVVKRLSALGLAAGDDKGNFNLDQTITRSEFATLMVRARGLEEAAKTATFNPFFKDVIATDWFQGFVSIAVSQDLAKGYPDKTFKPKNSVTYAEAATMLVRALGYEPLALQKGTWPNNFISQASELGISQGVTIDANKPAVRGDVFKMLDNSLRIDLMKQTTVGSQNNHEVLKGKNLLNEYLKVQVYDTKWTMTDRNKPEDLPSVSNVPVIGLGSLKYNEVELTSDSRSFFNKVKYKVADSINANDYAGQRVQVWIKDRDNTVVWMEGSEDETVVFDKYSALHISGKKLSPTKDKDQIKKDKFNKLTLELDSGTKYDFAEDVSVTFNFERATKASDVQEMLQKVLEADDAYSVKVVLNTKNEIRYISIVDDRSGDQNWVNNHFGSELIKKVTDKKITTLVGKDLALEDKKEGIDYLVFLDGKPAKLADLKEMDVYHAYRADGNKDKLLIFANRSTKEGKVEKVRSKSESDYRIVIDGKEYRFRNASFAKDKDKEIEDQITPDDIDRLEGTKVKVYLDAVGRIRHIEGYEANDRKQMAIISRSGTYDPYSDQYTFKVYTEKGNSKTVKLDGKYVKNEEGEKFSDLKKNESEKKIAEKYLTLSKDKNFILAQIELDASGDIEKVELKDTSKAKHMSKDQWKKDSDKDDETVKFGGKTYDFSSSSVVFDGSKGIQSGDVTSLKSPGTIKWSKIAEKEAEVYFTVDGSDVEAVFVVDSKGIASGSEYGVVVEAYRSSGVDKLKIKHRDDDGKWVEKEYKYDGDGKDKLEKTFIRFSADSDDLIDEDDVRVIVDARSDDEYNYKILKEKDYEDAGVNDVIAAQVDKIESKKIYFNTFDEKKQSIRLNKDVVYFDNEGKDLGEVDTDDYVVLVDTDNDSDSIDYVLYVISEDDAKTNGYLKKDDNKETLMDQFMKQNWKDDKPDPGEDDSDIDFTSSNYEKYPGETYLYAVAGTLKGEAKDADEVKVEIGSESKTVTVKDGAKFSAGLFVDTLEKTATVTAYKDGEKLYSTEVKLSAK

(19) United States
(12) Patent Application Publication
(10) Pub. No.: US 2024/0199710 A1
(43) Pub. Date: Jun. 20, 2024

(54) BIOACTIVE POLYPEPTIDES AND METHODS RELATED THERETO

(71) Applicant: ECOLIBRIUM BIOLOGICALS HOLDINGS LIMITED, Pukekohe (NZ)

(72) Inventors: Stephen Reynold FORD, Pukekohe (NZ); Mildred Marsha ORMSKIRK, Pukekohe (NZ); Travis GLARE, Pukekohe (NZ)

(21) Appl. No.: 18/557,095

(22) PCT Filed: Aug. 24, 2020
(86) PCT No.: PCT/NZ2020/050092

§ 371 Date: Oct. 25, 2023

(30) Foreign Application Priority Data
Aug. 23, 2019 (NZ) 756630

(51) Int. Cl. C07K 14/32; A01N 25/14; A01N 37/46; A01N 63/50; C12N 1/20; C12N 15/63; C12P 21/02; C12R 1/08

(52) U.S. Cl. CPC C07K 14/32; A01N 25/14; A01N 37/46; A01N 63/50; C12N 1/205; C12N 15/63; C12P 21/02; C12R 2001/08

(57) ABSTRACT

The invention relates to bioactive polypeptides from strains of Brevibacillus laterosporus which have useful activity including pesticidal activity such as insecticidal activity, compositions comprising said polypeptides, and methods for using the polypeptides and compositions, for example in methods of controlling agriculturally-important pests including insect pests.

### TECHNICAL FIELD

The invention relates to the identification and characterisation of bioactive polypeptides from strains of Brevibacillus laterosporus which have useful activity, including pesticidal activity such as insecticidal activity, compositions comprising said polypeptides, and methods for using the polypeptides and compositions, for example in methods of controlling agriculturally-important pests such as insect pests.

### BACKGROUND

The following includes information that may be useful in understanding the present inventions. It is not an admission that any of the information provided herein is prior art, or relevant, to the presently described or claimed inventions, or that any publication or document that is specifically or implicitly referenced is prior art. Any discussion of the prior art throughout the specification should in no way be considered as an admission that such prior art is widely known or forms part of the common general knowledge in the field.

Insect pests represent a significant economic cost to modern agriculture. Current systems of agriculture often require one or a few crops or plant types to be grown over a large area. Such an ecologically unbalanced system is susceptible to insect pressure. However, even more integrated production systems, such as those more closely emulating naturally-occurring environments, are susceptible to insect pests.

Some insect pests are also harmful to animal health, including human health. For example, mosquitos are known to carry a variety of diseases, and act as vectors in the spread of disease. Control of insect vectors of disease has thus been explored as a mechanism to control the incidence and distribution of disease.

Traditionally, control of insect pests has been pursued through the use of chemical insecticides and pesticides. However, consumers are becoming increasingly concerned about chemical residues and their effects on animal and plant health, and the environment. Moreover, many insect pests are becoming resistant to pesticides and insecticides.

Biological control represents an alternative means of controlling insect pests which reduces dependence on chemicals. Such methods enjoy greater public acceptance, and may be more effective and sustainable than chemical control methods.

A wide range of biological control agents including bacteria, yeast and fungi have been investigated for use in controlling insect pests. One widely investigated genus of bacteria for insecticidal use is Bacillus.

Bacillus is a genus containing many diverse bacterial species with diverse properties, varying from detrimental to animal and plant health, to useful for insect control. For example, Bacillus thuringiensis (Bt) in particular, is a well known biocontrol agent commercially available in products such as Thuricide® and Dipel®.

However, there has been reports of insect resistance to Bt developing. See for example Tabashnik et al (1990); Baxter et al (2011); and Tabashnik et al (1998).

Accordingly, there remains a need for alternatives to existing pesticidal and insecticidal treatments, including existing biocontrol treatments. The Brevibacillus laterosporus polypeptides and compositions provided herein go at least some way to meeting this need.

One object of the present invention is therefore to go some way towards overcoming one or more of the deficiencies identified above, and/or provide novel agents and compositions useful as a biocontrol agent, and/or a method for producing and/or using such agents, and/or to at least provide the public with a useful choice.

### SUMMARY OF THE INVENTION

According to a first aspect the invention relates to a method for controlling one or more insect pests, comprising the step of applying to a plant or its surroundings or a locus at which insect pests are or may become present a composition comprising one or more polypeptides selected from the group comprising:
a) a polypeptide comprising or consisting of the amino acid sequence depicted in any one of Sequence ID No.s 1 to 87;
b) a polypeptide comprising or consisting of the amino acid sequence corresponding to residues 89 to 322 of Sequence ID No. 1;
c) a polypeptide comprising or consisting of the amino acid sequence corresponding to residues 91 to 322 of Sequence ID No. 1;
d) a polypeptide comprising or consisting of the amino acid sequence corresponding to residues 147 to 322 of Sequence ID No. 1;
e) a polypeptide comprising or consisting of the amino acid sequence corresponding to residues 74 to 307 of Sequence ID No. 32;
f) a polypeptide comprising or consisting of the amino acid sequence corresponding to residues 76 to 307 of Sequence ID No. 32;
g) a polypeptide comprising or consisting of the amino acid sequence corresponding to residues 132 to 307 of Sequence ID No. 32;
h) a polypeptide comprising or consisting of the amino acid sequence corresponding to residues 89 to 322 of Sequence ID No. 58;
i) a polypeptide comprising or consisting of the amino acid sequence corresponding to residues 91 to 322 of Sequence ID No. 58;
j) a polypeptide comprising or consisting of the amino acid sequence corresponding to residues 147 to 322 of Sequence ID No. 58;
k) a polypeptide comprising or consisting of at least 10 contiguous amino acids from any one of a) to j) above;
l) a polypeptide comprising or consisting of at least 20 contiguous amino acids from any one of a) to j) above;
m) a polypeptide comprising or consisting of at least about 10 contiguous amino acids from any one of a) to l) above, wherein said polypeptide has glycoside hydrolase activity;
n) a polypeptide comprising or consisting of at least about 10 contiguous amino acids from any one of a) to m) above, wherein said polypeptide has chitinase activity or one or more chitinase domains;
o) a polypeptide having at least about 90% amino acid identity to any one of a) to n) above;
p) a polypeptide having at least about 95% amino acid identity to any one of a) to n) above;
q) any combination of any two or more of a) to p) above.

In a second aspect, the invention relates to a method for controlling one or more pests, comprising the step of contacting the one or more pests with a pesticidally-effective amount of one or more polypeptides or a composition comprising one or more polypeptides, wherein the one or more polypeptides are selected from the group comprising:
a) a polypeptide comprising or consisting of the amino acid sequence depicted in any one of Sequence ID No.s 1 to 87;
b) a polypeptide comprising or consisting of the amino acid sequence corresponding to residues 89 to 322 of Sequence ID No. 1;
c) a polypeptide comprising or consisting of the amino acid sequence corresponding to residues 91 to 322 of Sequence ID No. 1;
d) a polypeptide comprising or consisting of the amino acid sequence corresponding to residues 147 to 322 of Sequence ID No. 1;
e) a polypeptide comprising or consisting of the amino acid sequence corresponding to residues 74 to 307 of Sequence ID No. 32;
f) a polypeptide comprising or consisting of the amino acid sequence corresponding to residues 76 to 307 of Sequence ID No. 32;
g) a polypeptide comprising or consisting of the amino acid sequence corresponding to residues 132 to 307 of Sequence ID No. 32;
h) a polypeptide comprising or consisting of the amino acid sequence corresponding to residues 89 to 322 of Sequence ID No. 58;
i) a polypeptide comprising or consisting of the amino acid sequence corresponding to residues 91 to 322 of Sequence ID No. 58;
j) a polypeptide comprising or consisting of the amino acid sequence corresponding to residues 147 to 322 of Sequence ID No. 58;
k) a polypeptide comprising or consisting of at least 10 contiguous amino acids from any one of a) to j) above;
l) a polypeptide comprising or consisting of at least 20 contiguous amino acids from any one of a) to j) above;
m) a polypeptide comprising or consisting of at least about 10 contiguous amino acids from any one of a) to l) above, wherein said polypeptide has glycoside hydrolase activity;
n) a polypeptide comprising or consisting of at least about 10 contiguous amino acids from any one of a) to m) above, wherein said polypeptide has chitinase activity or one or more chitinase domains;
o) a polypeptide having at least about 90% amino acid identity to any one of a) to n) above;
p) a polypeptide having at least about 95% amino acid identity to any one of a) to n) above;
q) any combination of any two or more of a) to p) above.

In a third aspect the present invention relates to a method of treating or protecting a plant or its surroundings, and/or plant derived materials, from pest infestation, wherein the method comprises applying to the plant or its surroundings a composition comprising an effective amount of one or more polypeptides selected from the group comprising:
a) a polypeptide comprising or consisting of the amino acid sequence depicted in any one of Sequence ID No.s 1 to 87;
b) a polypeptide comprising or consisting of the amino acid sequence corresponding to residues 89 to 322 of Sequence ID No. 1;
c) a polypeptide comprising or consisting of the amino acid sequence corresponding to residues 91 to 322 of Sequence ID No. 1;
d) a polypeptide comprising or consisting of the amino acid sequence corresponding to residues 147 to 322 of Sequence ID No. 1;
e) a polypeptide comprising or consisting of the amino acid sequence corresponding to residues 74 to 307 of Sequence ID No. 32;
f) a polypeptide comprising or consisting of the amino acid sequence corresponding to residues 76 to 307 of Sequence ID No. 32;
g) a polypeptide comprising or consisting of the amino acid sequence corresponding to residues 132 to 307 of Sequence ID No. 32;
h) a polypeptide comprising or consisting of the amino acid sequence corresponding to residues 89 to 322 of Sequence ID No. 58;
i) a polypeptide comprising or consisting of the amino acid sequence corresponding to residues 91 to 322 of Sequence ID No. 58;
j) a polypeptide comprising or consisting of the amino acid sequence corresponding to residues 147 to 322 of Sequence ID No. 58;
k) a polypeptide comprising or consisting of at least 10 contiguous amino acids from any one of a) to j) above;
l) a polypeptide comprising or consisting of at least 20 contiguous amino acids from any one of a) to j) above;
m) a polypeptide comprising or consisting of at least about 10 contiguous amino acids from any one of a) to l) above, wherein said polypeptide has glycoside hydrolase activity;
n) a polypeptide comprising or consisting of at least about 10 contiguous amino acids from any one of a) to m) above, wherein said polypeptide has chitinase activity or one or more chitinase domains;
o) a polypeptide having at least about 90% amino acid identity to any one of a) to n) above;
p) a polypeptide having at least about 95% amino acid identity to any one of a) to n) above;
q) any combination of any two or more of a) to p) above.

According to another aspect the present invention relates to a method of controlling and/or preventing a pest infestation characterised by the step of applying a composition comprising an effective amount of one or more polypeptides to a surface, wherein one or more of the polypeptides is selected from the group comprising:
a) a polypeptide comprising or consisting of the amino acid sequence depicted in any one of Sequence ID No.s 1 to 87;
b) a polypeptide comprising or consisting of the amino acid sequence corresponding to residues 89 to 322 of Sequence ID No. 1;
c) a polypeptide comprising or consisting of the amino acid sequence corresponding to residues 91 to 322 of Sequence ID No. 1;
d) a polypeptide comprising or consisting of the amino acid sequence corresponding to residues 147 to 322 of Sequence ID No. 1;
e) a polypeptide comprising or consisting of the amino acid sequence corresponding to residues 74 to 307 of Sequence ID No. 32;
f) a polypeptide comprising or consisting of the amino acid sequence corresponding to residues 76 to 307 of Sequence ID No. 32;
g) a polypeptide comprising or consisting of the amino acid sequence corresponding to residues 132 to 307 of Sequence ID No. 32;
h) a polypeptide comprising or consisting of the amino acid sequence corresponding to residues 89 to 322 of Sequence ID No. 58;
i) a polypeptide comprising or consisting of the amino acid sequence corresponding to residues 91 to 322 of Sequence ID No. 58;
j) a polypeptide comprising or consisting of the amino acid sequence corresponding to residues 147 to 322 of Sequence ID No. 58;
k) a polypeptide comprising or consisting of at least 10 contiguous amino acids from any one of a) to j) above;
l) a polypeptide comprising or consisting of at least 20 contiguous amino acids from any one of a) to j) above;
m) a polypeptide comprising or consisting of at least about 10 contiguous amino acids from any one of a) to l) above, wherein said polypeptide has glycoside hydrolase activity;
n) a polypeptide comprising or consisting of at least about 10 contiguous amino acids from any one of a) to m) above, wherein said polypeptide has chitinase activity or one or more chitinase domains;
o) a polypeptide having at least about 90% amino acid identity to any one of a) to n) above;
p) a polypeptide having at least about 95% amino acid identity to any one of a) to n) above;
q) any combination of any two or more of a) to p) above.

The invention further relates to methods of using a polypeptide or a composition comprising one or more polypeptides for the control of pests, particularly plant pests, such as insects or nematodes, wherein one or more of the polypeptides is selected from the group comprising:
a) a polypeptide comprising or consisting of the amino acid sequence depicted in any one of Sequence ID No.s 1 to 87;
b) a polypeptide comprising or consisting of the amino acid sequence corresponding to residues 89 to 322 of Sequence ID No. 1;
c) a polypeptide comprising or consisting of the amino acid sequence corresponding to residues 91 to 322 of Sequence ID No. 1;
d) a polypeptide comprising or consisting of the amino acid sequence corresponding to residues 147 to 322 of Sequence ID No. 1;
e) a polypeptide comprising or consisting of the amino acid sequence corresponding to residues 74 to 307 of Sequence ID No. 32;
f) a polypeptide comprising or consisting of the amino acid sequence corresponding to residues 76 to 307 of Sequence ID No. 32;
g) a polypeptide comprising or consisting of the amino acid sequence corresponding to residues 132 to 307 of Sequence ID No. 32;
h) a polypeptide comprising or consisting of the amino acid sequence corresponding to residues 89 to 322 of Sequence ID No. 58;
i) a polypeptide comprising or consisting of the amino acid sequence corresponding to residues 91 to 322 of Sequence ID No. 58;
j) a polypeptide comprising or consisting of the amino acid sequence corresponding to residues 147 to 322 of Sequence ID No. 58;
k) a polypeptide comprising or consisting of at least 10 contiguous amino acids from any one of a) to j) above;
l) a polypeptide comprising or consisting of at least 20 contiguous amino acids from any one of a) to j) above;
m) a polypeptide comprising or consisting of at least about 10 contiguous amino acids from any one of a) to l) above, wherein said polypeptide has glycoside hydrolase activity;
n) a polypeptide comprising or consisting of at least about 10 contiguous amino acids from any one of a) to m) above, wherein said polypeptide has chitinase activity or one or more chitinase domains;
o) a polypeptide having at least about 90% amino acid identity to any one of a) to n) above;
p) a polypeptide having at least about 95% amino acid identity to any one of a) to n) above;
q) any combination of any two or more of a) to p) above.

For example, the invention also relates to methods of controlling a pest population. The methods generally involve contacting the pests or the pest population with a pesticidally-effective amount of one or more of the polypeptides or a composition comprising one or more of the polypeptides as described herein. Such methods may be used to kill or reduce the numbers of target pests in a given area, or may be prophylactically applied to a locus, such as an environmental area, to prevent infestation by a susceptible pest.

In various embodiments, any of the methods described herein comprise the use of a water dispersible granule (WDG) formulation as herein described.

The invention further relates to the use of a composition as described herein for the control of one or more pests, including one or more insect or nematode pests.

The use of one or more of the polypeptides described herein, and/or of a composition comprising one or more of the polypeptides as described herein, in the manufacture of a formulation for the control of one or more pests is similarly contemplated.

In agricultural and horticultural applications, the invention is applicable to any plant or its surroundings. Illustrative plants are monocotyledonous or dicotyledonous plants such as alfalfa, barley, canola, corn (maize), cotton, flax, kapok, peanut, potato, oat, rice, rye, sorghum, soybean, sugarbeet, sugarcane, sunflower, tobacco, tomato, wheat, turf grass, pasture grass, berry, fruit, legume, vegetable, for example, capsicum, a cucurbit such as cucumber, onion, ornamental plants, shrubs, cactuses, succulents, and trees.

In further illustrative embodiments, the plant may be any plant, including plants selected from the order Solanales, including plants from the following families: Convolvulaceae, Hydroleaceae, Montiniaceae, Solanaceae, and Sphenocleaceae, and plants from the order Asparagales, including plants from the following families:

Amaryllidaceae, Asparagaceae, Asteliaceae, Blandfordiaceae, Boryaceae, Doryanthaceae, Hypoxidaceae, Iridaceae, Ixioliriaceae, Lanariaceae, Orchidaceae, Tecophilaeaceae, Xanthorrhoeaceae, and Xeronemataceae.

In another aspect the invention relates to a plant or part thereof treated with, or to which has been applied, a composition as described herein.

In one embodiment the plant or part thereof is reproductively viable, for example, a seed, bulb or cutting or other plant part capable of propagation.

In a further aspect the invention relates to an isolated, purified, recombinant or synthetic polypeptide selected from the group comprising:
a) a polypeptide comprising or consisting of the amino acid sequence depicted in any one of Sequence ID No.s 1 to 87;
b) a polypeptide comprising or consisting of the amino acid sequence corresponding to residues 89 to 322 of Sequence ID No. 1;
c) a polypeptide comprising or consisting of the amino acid sequence corresponding to residues 91 to 322 of Sequence ID No. 1;
d) a polypeptide comprising or consisting of the amino acid sequence corresponding to residues 147 to 322 of Sequence ID No. 1;
e) a polypeptide comprising or consisting of the amino acid sequence corresponding to residues 74 to 307 of Sequence ID No. 32;
f) a polypeptide comprising or consisting of the amino acid sequence corresponding to residues 76 to 307 of Sequence ID No. 32;
g) a polypeptide comprising or consisting of the amino acid sequence corresponding to residues 132 to 307 of Sequence ID No. 32;
h) a polypeptide comprising or consisting of the amino acid sequence corresponding to residues 89 to 322 of Sequence ID No. 58;
i) a polypeptide comprising or consisting of the amino acid sequence corresponding to residues 91 to 322 of Sequence ID No. 58;
j) a polypeptide comprising or consisting of the amino acid sequence corresponding to residues 147 to 322 of Sequence ID No. 58;
k) a polypeptide comprising or consisting of at least 10 contiguous amino acids from any one of a) to j) above;
l) a polypeptide comprising or consisting of at least 20 contiguous amino acids from any one of a) to j) above;
m) a polypeptide comprising or consisting of at least about 10 contiguous amino acids from any one of a) to l) above, wherein said polypeptide has glycoside hydrolase activity;
n) a polypeptide comprising or consisting of at least about 10 contiguous amino acids from any one of a) to m) above, wherein said polypeptide has chitinase activity or one or more chitinase domains;
o) a polypeptide having at least about 90% amino acid identity to any one of a) to n) above;
p) a polypeptide having at least about 95% amino acid identity to any one of a) to n) above;
q) any combination of any two or more of a) to p) above.

Any of the embodiments described herein can relate to any of the aspects presented herein.

Another aspect of the present invention relates to a composition, including a pharmaceutical or agricultural composition, comprising one or more polypeptides selected from the group comprising:
a) a polypeptide comprising or consisting of the amino acid sequence depicted in any one of Sequence ID No.s 1 to 87;
b) a polypeptide comprising or consisting of the amino acid sequence corresponding to residues 89 to 322 of Sequence ID No. 1;
c) a polypeptide comprising or consisting of the amino acid sequence corresponding to residues 91 to 322 of Sequence ID No. 1;
d) a polypeptide comprising or consisting of the amino acid sequence corresponding to residues 147 to 322 of Sequence ID No. 1;
e) a polypeptide comprising or consisting of the amino acid sequence corresponding to residues 74 to 307 of Sequence ID No. 32;
f) a polypeptide comprising or consisting of the amino acid sequence corresponding to residues 76 to 307 of Sequence ID No. 32;
g) a polypeptide comprising or consisting of the amino acid sequence corresponding to residues 132 to 307 of Sequence ID No. 32;
h) a polypeptide comprising or consisting of the amino acid sequence corresponding to residues 89 to 322 of Sequence ID No. 58;
i) a polypeptide comprising or consisting of the amino acid sequence corresponding to residues 91 to 322 of Sequence ID No. 58;
j) a polypeptide comprising or consisting of the amino acid sequence corresponding to residues 147 to 322 of Sequence ID No. 58;
k) a polypeptide comprising or consisting of at least 10 contiguous amino acids from any one of a) to j) above;
l) a polypeptide comprising or consisting of at least 20 contiguous amino acids from any one of a) to j) above;
m) a polypeptide comprising or consisting of at least about 10 contiguous amino acids from any one of a) to l) above, wherein said polypeptide has glycoside hydrolase activity;
n) a polypeptide comprising or consisting of at least about 10 contiguous amino acids from any one of a) to m) above, wherein said polypeptide has chitinase activity or one or more chitinase domains;
o) a polypeptide having at least about 90% amino acid identity to any one of a) to n) above;
p) a polypeptide having at least about 95% amino acid identity to any one of a) to n) above;
q) any combination of any two or more of a) to p) above;
together with a carrier.

In one embodiment, the composition comprises a pharmaceutically acceptable carrier. In one embodiment, the composition comprises an agriculturally acceptable carrier.

In certain embodiments, the composition comprises an extract or composition enriched in or to which has been added a sub-3 kDa fraction from B. laterosporus NMI No. V12/001945 or a culture thereof. In one embodiment, the extract or composition enriched in or to which has been added a sub-3 kDa fraction from B. laterosporus NMI No. V12/001945 or a culture thereof is substantially non-proteinaceous. In one embodiment, the sub-3 kDa fraction from B. laterosporus NMI No. V12/001945 or a culture thereof is substantially non-proteinaceous.

In certain embodiments, the composition comprises an extract or composition enriched in or to which has been added a sub-3 kDa fraction from B. laterosporus NMI No. V12/001944 or a culture thereof. In one embodiment, the extract or composition enriched in or to which has been added a sub-3 kDa fraction from B. laterosporus NMI No. V12/001944 or a culture thereof is substantially non-proteinaceous. In one embodiment, the sub-3 kDa fraction from B. laterosporus NMI No. V12/001944 or a culture thereof is substantially non-proteinaceous.

In certain embodiments, the composition comprises an extract or composition enriched in or to which has been added a sub-3 kDa fraction from B. laterosporus NMI No. V12/001946 or a culture thereof. In one embodiment, the extract or composition enriched in or to which has been added a sub-3 kDa fraction from B. laterosporus NMI No. V12/001946 or a culture thereof is substantially non-proteinaceous. In one embodiment, the sub-3 kDa fraction from B. laterosporus NMI No. V12/001946 or a culture thereof is substantially non-proteinaceous.

Another aspect of the present invention relates to a kit comprising a composition as described herein.

According to another aspect, the invention relates to an expression construct comprising a nucleic acid encoding a polypeptide selected from the group comprising:
a) a polypeptide comprising or consisting of the amino acid sequence depicted in any one of Sequence ID No.s 1 to 87;
b) a polypeptide comprising or consisting of the amino acid sequence corresponding to residues 89 to 322 of Sequence ID No. 1;
c) a polypeptide comprising or consisting of the amino acid sequence corresponding to residues 91 to 322 of Sequence ID No. 1;
d) a polypeptide comprising or consisting of the amino acid sequence corresponding to residues 147 to 322 of Sequence ID No. 1;
e) a polypeptide comprising or consisting of the amino acid sequence corresponding to residues 74 to 307 of Sequence ID No. 32;
f) a polypeptide comprising or consisting of the amino acid sequence corresponding to residues 76 to 307 of Sequence ID No. 32;
g) a polypeptide comprising or consisting of the amino acid sequence corresponding to residues 132 to 307 of Sequence ID No. 32;
h) a polypeptide comprising or consisting of the amino acid sequence corresponding to residues 89 to 322 of Sequence ID No. 58;
i) a polypeptide comprising or consisting of the amino acid sequence corresponding to residues 91 to 322 of Sequence ID No. 58;
j) a polypeptide comprising or consisting of the amino acid sequence corresponding to residues 147 to 322 of Sequence ID No. 58;
k) a polypeptide comprising or consisting of at least 10 contiguous amino acids from any one of a) to j) above;
l) a polypeptide comprising or consisting of at least 20 contiguous amino acids from any one of a) to j) above;
m) a polypeptide comprising or consisting of at least about 10 contiguous amino acids from any one of a) to l) above, wherein said polypeptide has glycoside hydrolase activity;
n) a polypeptide comprising or consisting of at least about 10 contiguous amino acids from any one of a) to m) above, wherein said polypeptide has chitinase activity or one or more chitinase domains;
o) a polypeptide having at least about 90% amino acid identity to any one of a) to n) above;
p) a polypeptide having at least about 95% amino acid identity to any one of a) to n) above; q) any combination of any two or more of a) to p) above.

Another aspect of the present invention relates to a vector comprising an expression construct as described above.

Another aspect of the present invention relates to a host cell comprising an expression construct or a vector as defined above.

In a further aspect, the present invention relates to the use of a purified, isolated, recombinant or synthetic polypeptide to control one or more pests, wherein the polypeptide is selected from the group comprising:
a) a polypeptide comprising or consisting of the amino acid sequence depicted in any one of Sequence ID No.s 1 to 87;
b) a polypeptide comprising or consisting of the amino acid sequence corresponding to residues 89 to 322 of Sequence ID No. 1;
c) a polypeptide comprising or consisting of the amino acid sequence corresponding to residues 91 to 322 of Sequence ID No. 1;
d) a polypeptide comprising or consisting of the amino acid sequence corresponding to residues 147 to 322 of Sequence ID No. 1;
e) a polypeptide comprising or consisting of the amino acid sequence corresponding to residues 74 to 307 of Sequence ID No. 32;
f) a polypeptide comprising or consisting of the amino acid sequence corresponding to residues 76 to 307 of Sequence ID No. 32;
g) a polypeptide comprising or consisting of the amino acid sequence corresponding to residues 132 to 307 of Sequence ID No. 32;
h) a polypeptide comprising or consisting of the amino acid sequence corresponding to residues 89 to 322 of Sequence ID No. 58;
i) a polypeptide comprising or consisting of the amino acid sequence corresponding to residues 91 to 322 of Sequence ID No. 58;
j) a polypeptide comprising or consisting of the amino acid sequence corresponding to residues 147 to 322 of Sequence ID No. 58;
k) a polypeptide comprising or consisting of at least 10 contiguous amino acids from any one of a) to j) above;
l) a polypeptide comprising or consisting of at least 20 contiguous amino acids from any one of a) to j) above;
m) a polypeptide comprising or consisting of at least about 10 contiguous amino acids from any one of a) to l) above, wherein said polypeptide has glycoside hydrolase activity;
n) a polypeptide comprising or consisting of at least about 10 contiguous amino acids from any one of a) to m) above, wherein said polypeptide has chitinase activity or one or more chitinase domains;
o) a polypeptide having at least about 90% amino acid identity to any one of a) to n) above;
p) a polypeptide having at least about 95% amino acid identity to any one of a) to n) above;
q) any combination of any two or more of a) to p) above.

Another aspect of the present invention relates to a method of preparing a pesticidal or insecticidal composition, the method comprising
a) optionally growing a culture of B. laterosporus NMI No. V12/001944, and/or B. laterosporus NMI No. V12/001945, and/or B. laterosporus NMI No. V12/001946;
b) providing a cellular extract obtained from B. laterosporus NMI No. V12/001944, and/or B. laterosporus NMI No. V12/001945, and/or B. laterosporus NMI No. V12/001946, and/or a composition comprising or derived from media in which B. laterosporus NMI No. V12/001944, and/or B. laterosporus NMI No. V12/001945, and/or B. laterosporus NMI No. V12/001946, is or has been grown, wherein the cellular extract or the composition comprises undenatured protein comprising one or more polypeptides as herein described;
c) admixing the cellular extract and/or composition comprising undenatured protein with one or more agriculturally-acceptable carriers;
to provide the pesticidal or insecticidal composition.

In various embodiments, the method comprises the additional step of admixing the cellular extract or composition with one or more of the polypeptides described herein. In one embodiment, the method comprises admixing the cellular extract or composition with a composition enriched in one or more of the polypeptides described herein.

In one embodiment, the cellular extract is prepared by subjecting one or more cells or spores from B. laterosporus NMI No. V12/001944, and/or B. laterosporus NMI No. V12/001945, and/or B. laterosporus NMI No. V12/001946 to proteolysis, for example, proteolysis for a period sufficient to remove one or more S-layer proteins as described herein from the bacterial cell or spore. In one embodiment, the proteolysis is for a period sufficient to remove one or more full-length, stable, and biologically-active S-layer proteins as described herein from the cell or spore surface. In one embodiment, the proteolysis is as herein described in the Examples.

Another aspect of the present invention relates to method of preparing a pesticidal or insecticidal composition, the method comprising
a) optionally growing one or more cells comprising a nucleic acid encoding one or more of the polypeptides as described herein, or an expression construct or vector as described herein, or one or more host cells as described herein, under conditions suitable for the expression of said one or more polypeptides;
b) providing a cellular extract from said one or more cells or one or more host cells, or a composition comprising or derived from media in which said one or more cells or host cells is or has been grown, wherein the cellular extract or composition comprises one or more of the polypeptides as described herein;
c) admixing the cellular extract and/or composition with one or more agriculturally-acceptable carriers;
to provide the pesticidal or insecticidal composition.

In one embodiment, the cellular extract is prepared by subjecting the one or more cells or host cells to proteolysis, for example, proteolysis for a period sufficient to remove one or more S-layer proteins as described herein from the cell. In one embodiment, the one or more cells or host cells is bacterial, and the proteolysis is for a period sufficient to remove one or more S-layer proteins as described herein from the cell surface. In one embodiment, the proteolysis is as herein described in the Examples.

In a further aspect, the present invention relates to a method of controlling a pest or pest population, the method comprising contacting the pest or pest population, or applying to a surface an effective amount of one or more polypeptides selected from the group comprising:
a) a polypeptide comprising or consisting of the amino acid sequence depicted in any one of Sequence ID No.s 1 to 87;
b) a polypeptide comprising or consisting of the amino acid sequence corresponding to residues 89 to 322 of Sequence ID No. 1;
c) a polypeptide comprising or consisting of the amino acid sequence corresponding to residues 91 to 322 of Sequence ID No. 1;
d) a polypeptide comprising or consisting of the amino acid sequence corresponding to residues 147 to 322 of Sequence ID No. 1;
e) a polypeptide comprising or consisting of the amino acid sequence corresponding to residues 74 to 307 of Sequence ID No. 32;
f) a polypeptide comprising or consisting of the amino acid sequence corresponding to residues 76 to 307 of Sequence ID No. 32;
g) a polypeptide comprising or consisting of the amino acid sequence corresponding to residues 132 to 307 of Sequence ID No. 32;
h) a polypeptide comprising or consisting of the amino acid sequence corresponding to residues 89 to 322 of Sequence ID No. 58;
i) a polypeptide comprising or consisting of the amino acid sequence corresponding to residues 91 to 322 of Sequence ID No. 58;
j) a polypeptide comprising or consisting of the amino acid sequence corresponding to residues 147 to 322 of Sequence ID No. 58;
k) a polypeptide comprising or consisting of at least 10 contiguous amino acids from any one of a) to j) above;
l) a polypeptide comprising or consisting of at least 20 contiguous amino acids from any one of a) to j) above;
m) a polypeptide comprising or consisting of at least about 10 contiguous amino acids from any one of a) to l) above, wherein said polypeptide has glycoside hydrolase activity;
n) a polypeptide comprising or consisting of at least about 10 contiguous amino acids from any one of a) to m) above, wherein said polypeptide has chitinase activity or one or more chitinase domains;
o) a polypeptide having at least about 90% amino acid identity to any one of a) to n) above;
p) a polypeptide having at least about 95% amino acid identity to any one of a) to n) above;
q) any combination of any two or more of a) to p) above.

In another aspect the invention relates to a plant or part thereof treated with, or to which has been applied, a composition as described herein.

In one embodiment the plant or part thereof is reproductively viable, for example, a seed, bulb or cutting or other plant part capable of propagation.

In various embodiments, the composition provided herein, for example, the insecticidal composition and/or the composition to be applied to control of pests, is formulated as a water dispersible granule (WDG).

In various embodiments, the water dispersible granule formulation comprises one or more polypeptides described above, for example an effective amount of one or more polypeptides described above, together with one or more of the following:
a) from about 2% to about 80% w/w viable B. laterosporus NMI No. V12/001944, and/or B. laterosporus NMI No. V12/001945, and/or B. laterosporus NMI No. V12/001946;
b) from about 2% to about 80% w/w cellular extract obtained from B. laterosporus NMI No. V12/001944, and/or B. laterosporus NMI No. V12/001945, and/or B. laterosporus NMI No. V12/001946;
c) from about 2% to about 80% w/w of a composition comprising or derived from media in which B. laterosporus NMI No. V12/001944, and/or B. laterosporus NMI No. V12/001945, and/or B. laterosporus NMI No. V12/001946 is or has been grown;
d) from about 1% to about 50% w/w one or more wetting agent;
e) from about 1% to about 50% w/w one or more dispersant;
f) from about 2% to about 50% w/w one or more humectant or agent to control water activity;
g) from about 0% to about 50% w/w one or more protectants;
h) from about 0% to about 50% w/w one or more nutrients or mixture thereof;
i) from about 5% to about 80% w/w one or more filler;
j) from about 0% to about 20% w/w one or more antioxidant or UV radiation protectant;
k) from about 1% to about 50% w/w one or more binding agent;
l) from about 0% to about 50% one or more disintegrating agent;
m) from about 0% to about 10% w/w water;
n) any combination of two or more of any of a) to m) above.

In various embodiments, the water dispersible granule formulation comprises at least about 0.01% w/w one or more polypeptides described above, together with one or more of the following:
a) from about 2% to about 80% w/w viable B. laterosporus NMI No. V12/001944, and/or B. laterosporus NMI No. V12/001945, and/or B. laterosporus NMI No. V12/001946;
b) from about 2% to about 80% w/w cellular extract obtained from B. laterosporus NMI No. V12/001944, and/or B. laterosporus NMI No. V12/001945, and/or B. laterosporus NMI No. V12/001946;
c) from about 2% to about 80% w/w of a composition comprising or derived from media in which B. laterosporus NMI No. V12/001944, and/or B. laterosporus NMI No. V12/001945, and/or B. laterosporus NMI No. V12/001946 is or has been grown;
d) from about 1% to about 50% w/w one or more wetting agent;
e) from about 1% to about 50% w/w one or more dispersant;
f) from about 2% to about 50% w/w one or more humectant or agent to control water activity;
g) from about 0% to about 50% w/w one or more protectants;
h) from about 0% to about 50% w/w one or more nutrients or mixture thereof;
i) from about 5% to about 80% w/w one or more filler;
j) from about 0% to about 20% w/w one or more antioxidant or UV radiation protectant;
k) from about 1% to about 50% w/w one or more binding agent;
l) from about 0% to about 50% one or more disintegrating agent;
m) from about 0% to about 10% w/w water;
n) any combination of two or more of any of a) to m) above.

In various embodiments, the polypeptide described herein is administered to the subject at a dosage of from about 1 ng/kg to about 1 g/kg. In various embodiments, the polypeptide described herein is administered to the subject at a dosage of from about 1 ng/kg to about 100 mg/kg, or from about 1 ng/kg to about 10 mg/kg. For example, the polypeptide described herein is administered at a dosage of from about 1 ng/kg to about 100 μg/kg, or from about 1 ng/kg to about 10 μg/kg, 1 ng/kg to about 1 μg/kg, or from about 1 ng/kg to about 100 ng/kg.

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9 and 10) and also any range of rational numbers within that range (for example, 2 to 8, 1.5 to 5.5 and 3.1 to 4.7). These are only examples of what is specifically intended and all possible combinations of numerical values between the lowest value and the highest value enumerated are to be considered to be expressly stated in this application in a similar manner.

Those skilled in the art will appreciate the meaning of various terms of degree used herein. For example, as used herein in the context of referring to an amount (e.g., “about 9%”), the term “about” represents an amount close to and including the stated amount that still performs a desired function or achieves a desired result, e.g. “about 9%” can include 9% and amounts close to 9% that still perform a desired function or achieve a desired result. For example, the term “about” can refer to an amount that is within less than 10% of, within less than 5% of, within less than 1% of, within less than 0.1% of, or within less than 0.01% of the stated amount. It is also intended that where the term “about” is used, for example with reference to a figure, concentration, amount, integer or value, the exact figure, concentration, amount, integer or value is also specifically contemplated.

Other objects, aspects, features and advantages of the present invention will become apparent from the following description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIG. 1 is a schematic overview of the Bl45 sporulated culture methanol extraction. A| 40 ml Bl45 sporulated culture. B| Metabolic quenching of the sporulated culture with quenching buffer at −40° C. C| Lyophilised culture supernatant derived from a 40 ml culture. D| Methanol extraction of the spore pellet derived from 10 ml of culture at −80° C. E| Lyophilised intracellular extract derived from the spores of the 40 ml culture F| Methanol extracted spore pellet derived from the 40 ml sporulated culture. G| Lyophilised intracellular extract suspended in 4 ml ammonium acetate buffer. H|Methanol extracted spore pellet resuspended in 4 ml ammonium acetate buffer. I| Lyophilised quenched supernatant suspended in 4 ml ammonium acetate.

FIG. 2 is a chromatograph of High performance liquid size exclusion chromatography of Bl45 culture supernatant ammonium sulphate precipitate. Four distinct areas were selected in the first peak for protein analysis by SDS-PAGE and Native PAGE (FIGS. 3 & 4). These were fractions 23-28, fractions 29-32, fractions 33-38 and fractions 39-46. Fractions 47-69 were selected for protein analysis in the second peak.

FIG. 3 is a photo of SDS-PAGE of Bl45 supernatant size exclusion chromatography fractions.

FIG. 4 is a photo of native PAGE of Bl45 size exclusion chromatography fractions. A lower number of bands were visible in most samples compared to the SDS-PAGE gel (FIG. 3). Fractions 26-36, 37-39, 40-42, 43-47 and 48-67 were pooled to test for DBM larvae activity (Table 2).

FIG. 5 is a photo of native PAGE of the size exclusion chromatography bioassay treatments. All size exclusion fractions had different protein profiles. The last size exclusion fractions 47 to 67 were derived from the second peak in the chromatograph (FIG. 2).

FIG. 6 is a chromatograph of Reversed Phase High Pressure Liquid Chromatography of Bl45 culture supernatant. The HPLC yielded five separate peaks denoted 1 to 5 in red bold numbers. The fractions representing each peak were pooled and tested for toxicity in a DBM bioassay (Table 7).

FIG. 7 is a photo of SDS-PAGE of the treatments applied in DBM bioassay repeat one of three, reveals two unique putative diamondback moth (DBM) larvicidal protein bands in the quenched supernatant. The red and green arrows show the protein bands that were unique in the highly insecticidal quenched supernatant. These unique proteins were selected as putative toxins to be further analysed and identified by ESI-mass spectrometry. The approximate protein weights of the bands indicated by the red and green arrows were about 60 kDa and 40 kDa respectively. The gel concentration was 10% acrylamide/bis.
^{a}|50 mM ammonium acetate. ^{b}| Dipel Bt subsp. kurstaki 32000 U/ml (1 mg/ml) ^{c}|1:10 dilution.

FIG. 8 is a photo of SDS-PAGE of the treatments applied in the DBM bioassay repeat two of three showing two unique putative DBM larvicidal protein bands in the quenched supernatant. The red and green arrows show the protein bands that were unique in the highly insecticidal quenched supernatant. These unique proteins were selected as putative toxins to be further analysed and identified by ESI-mass spectrometry. The approximate protein weights of the bands indicated by the red and green arrows were about 60 kDa and 40 kDa respectively. The gel concentration was 8% acrylamide/bis.
^{a}| mLB⁺ and 50 mM ammonium acetate. ^{b}| Dipel Bt subsp. kurstaki 32000 U/ml (1 mg/ml). ^{c}|1:10 dilution. ^{d}|1:20 dilution.

FIG. 9 is a photo of SDS-PAGE of the treatments applied in the DBM bioassay repeat three of three reveals three unique putative DBM larvicidal protein bands in the quenched supernatant. The red, orange and green arrows show the protein bands that were unique in the highly insecticidal quenched supernatant. These unique proteins were selected as putative toxins to be further analysed and identified by ESI-mass spectrometry. The approximate protein weights of the bands indicated by the red, orange and green arrows were about 60 kDa, 50 kDa and 40 kDa respectively. The gel concentration was 8% acrylamide/bis. ^{a}|mLB⁺ and 50 mM ammonium acetate. ^{b}| 10× concentrated mLB⁺ and 50 mM ammonium acetate. ^{c}| 50 mM ammonium acetate; pH 8.5. ^{d}| Dipel^{DF} Bt subsp. kurstaki 32000 U/ml (1 mg/ml). ^{e}| 1:10 dilution. ^{f}| 1:5 dilution.

FIG. 10 is a graphical depiction of predicted Open Reading Frame (ORF) in the Bl45 genome encoding the putative toxin encoding surface layer protein and putative conserved domains in the surface layer encoding gene. A| Predicted ORF of S-layer encoding gene in the Bl45 genome, highlighted in blue. Identified in Geneious 8.1.7 by the mass spectrometry acquired peptide sequence, RALGYEPLALQKG. The amino acid sequence was in frame with the predicted ORF, reverse frame 1. The ORF contained 3318 nucleotide bases. The translated amino acid sequence contains 1105 residues and the predicted molecular weight is 124.1 kDa. B| Predicted surface layer homology (SLH) domains identified in the S-layer amino acid sequence using DELTA-BLAST (NCBI). C| Predicted conserved 235 kDa rhoptry protein family domain identified in the S-layer nucleotide sequence using Blastx (NCBI).

FIG. 11 is a graphical depiction of Swiss-model analysis of surface layer amino acid sequence from Brevibacillus laterosporus NMI VM12/001945. Green; amino acid sequence of S-layer protein from Bl45. Yellow; sequence homology detected to structurally elucidated Sap S-layer protein SLH-domain from Bacillus anthracis. Blue; sequence homology detected to structurally elucidated ChiW chitinase from Paenibacillus species. Pink; sequence homology detected to structurally elucidated CelS cellobiohydrolase protein from Clostridium thermocellum.

FIG. 12 is a graphical depiction of predicted Open Reading Frames flanking the S-layer toxin encoding gene of Brevibacillus laterosporus NMI VM12/001945. Predicted in Geneious version 8.1.7. Left Open Reading Frame|Potential adhesin. Contains 2577 nucleotide bases and is encoded in reverse frame 3, standard genetic code. The amino acid translation contains 858 residues with a predicted protein mass of 88.8 kDa. Right Open Reading Frame|Potential efflux pump. Contains 1197 nucleotide bases and is encoded in reverse frame 3, standard genetic code. The amino acid translation contains 398 residues with a predicted protein mass of 43.9 kDa.

FIG. 13 is a graphical depiction of potential conserved domains located in the hypothetical adhesion/fimbriae-like protein from Brevibacillus laterosporus NMI VM12/001945. A| BLASTx search. The PRK12806, Mfa_like_1 and Hia multidomains were detected as putative conserved domains within the ORF (Table 23). B| DELTA-BLAST. Two possible conserved domains were detected within the translated amino acid sequence, the DUF4815 superfamily and the flgK domain (Table 23).

FIG. 14 is a graphical depiction of Swiss-model analysis of putative adhesin/fimbriae-like amino acid sequence from Brevibacillus laterosporus NMI VM12/001945. Green; amino acid sequence of putative adhesin/fimbriae-like protein from Bl45. Yellow; sequence homology detected to structurally elucidated SbsC S-layer protein from Geobacillus stearothermophilus. Pink; sequence homology detected to structurally elucidated SiiE adhesin from Salmonella enterica. Blue; sequence homology detected to structurally elucidated MpAFP adhesin from Marinomonas primoyensii. Orange; sequence homology detected to structurally elucidated CopC protein from Pseudomonas syringae. Red; sequence homology detected to structurally elucidated ClfB from Staphylococcus aureus.

FIG. 15 is a graphical depiction showing putative conserved domains of the putative virulent transporter protein from Brevibacillus laterosporus NMI VM12/001945. A| BLASTx search. Three potential functional domains were identified. Outer membrane efflux protein (OEP) superfamily, TolC (Table 27). B| DELTA-BLAST search. Three potential functional domains were identified. Outer membrane efflux protein (OEP) superfamily and TolC (Table 27).

FIG. 16 is a graphical depiction of Swiss-model analysis of putative outer membrane efflux protein amino acid sequence from Brevibacillus laterosporus NMI VM12/001945. Green; amino acid sequence of putative outer membrane efflux protein from Bl45. Yellow; sequence homology detected to structurally elucidated ST50 outer membrane protein from Salmonella enterica typhi. Pink; sequence homology detected to structurally elucidated CusC outer membrane protein of Escherichia coli. Blue; sequence homology detected to structurally elucidated TolC outer membrane efflux protein from E. coli. Orange; sequence homology detected to structurally elucidated OprN outer membrane protein factor from Pseudomonas aeruginosa.

FIG. 17 is a photo of SDS-PAGE of a time course of protein expression in Bl45, as described in Example 8 herein. Lane 1, MW ladder; Lane 2, 0 hours; Lane 3, 6 hours; Lane 4, 23 hours; Lane 5, 30 hours; Lane 6, 46 hours; Lane 7, 72 hours; Lane 8, 96 hours.

FIG. 18 is a graph depicting the results of a DBM bioassay as described herein in Example 8. % Unwellness of DBM caterpillars of heated and unheated biomass harvested at 30 hr, 72 hr and 96 hr over a 6-day trial period is shown.

FIG. 19 is a photo of SDS-PAGE analysis of heated and unheated pellet samples (30 hr, 72 hr and 96 hr) after 5 extensive washes, as described in Example 8 herein. Lane 1, MW ladder; Lane 2, 30 hour, unheated; Lane 3, 72 hours, unheated; Lane 4, 96 hours, unheated; Lane 5, 30 hours, heated; Lane 6, 72 hours, heated; Lane 7, 96 hours, heated.

FIG. 20 is a graph showing the results of bioassays on partially purified S-layer protein as described herein in Example 8, in which % unwellness of DBM caterpillars of heated and unheated pellet and purified S-layer protein samples over a 5-day trial period is shown.

FIG. 21 is a graph showing the results of bioassays on the insecticidal activity of various B. laterosporus NMI No. V12/001946 compositions as described herein in Example 9.

FIG. 22 is a graph showing the results of bioassays on the insecticidal activity of various B. laterosporus NMI No. V12/001944 compositions as described herein in Example 10.

### DETAILED DESCRIPTION

In one aspect, the present invention is directed to one or more polypeptides from Brevibacillus laterosporus strains, wherein the one or more polypeptides have activity against one or more insect pests. The invention further relates to compositions comprising said polypeptides, particularly to insecticidal compositions, including a composition that has insecticidal activity against one or more insect pests of agricultural and horticultural significance.

The term “and/or” can mean “and” or “or”.

The term “agriculturally acceptable carrier” covers all liquid and solid carriers known in the art such as water and oils, as well as adjuvants, dispersants, binders, wettants, surfactants, humectants, protectants, UV protectants and/or stabilisers, tackifiers, and the like that are ordinarily known for use in the preparation of agricultural compositions, including insecticide compositions.

The term “biologically pure culture” or “biologically pure isolate” as used herein refers to a culture, for example of a B. laterosporus strain as described herein, comprising at least 99% and more preferably at least 99.5% cells of the specified strain. Typically, a biologically pure culture or a biologically pure isolate is an axenic culture or an axenic isolate.

As used herein the term “cellular extract” refers to a substance or mixture of substances obtained from a cell, typically in this description a bacterial cell.

It should be appreciated that the ‘cellular extract’ may be obtained in a variety of different ways, and may come in a variety of different forms without departing from the scope of the present invention.

In some embodiments the cellular extract may be a crude extract of the contents of the cell. For example, in certain embodiments the crude extract is obtained via concentration of the cells, for example by centrifugation of a whole broth culture, followed by resuspension in a suitable buffer, typically followed by cellular lysis.

Such an extract may have been derived by various well known methods of cell lysis, including, for example, sonication, osmotic lysis, enzymatic lysis, lysis using a French press or a Mantin gaulin press, or particle or bead-mediated lysis.

As used herein the term “sonicate” or grammatical variants thereof refers to subjecting a cell to ultrasonic vibrations in order to fragment the cell wall to release the contents of the cell.

In other embodiments the cellular extract is a freeze dried or a spray dried extract. In certain embodiments, the freeze or spray dried extract is obtained via any cellular extract which has also been subjected to a freeze-or spray drying process as are well known in the art.

In preferred embodiments the cellular extract may be derived from the aforementioned methods via sonication; French press; Mantin gaulin press, bead basher, bead mill mincer osmotic lysis or enzyme related lysis.

The term “comprising” as used in this specification means “consisting at least in part of”. When interpreting each statement in this specification that includes the term “comprising”, features other than that or those prefaced by the term may also be present. Related terms such as “comprise” and “comprises”, and the terms “including”, “include” and “includes” are to be interpreted in the same manner.

The term “consisting essentially of” when used in this specification refers to the features stated and allows for the presence of other features that do not materially alter the basic characteristics of the features specified.

The term “contacting” as used herein refers to the provision of a composition or strain(s) as described herein to a pest in a manner useful to effect pest control. Most commonly contacting will involve the pest feeding on material comprising a composition or strain(s) as described herein but is not limited thereto. Accordingly, “contacting” includes feeding.

The term “control” or “controlling” as used herein generally comprehends preventing an increase in, reducing, or eradicating a population or one or more members of a population, or preventing, reducing or eradicating infection or infestation by one or more pests or pathogens, such as infection by one or more phytopathogens or pests, or inhibiting the rate and extent of such infection, such as reducing a pest population at a locus, for example in or on a plant or its surroundings, wherein such prevention or reduction in the infection(s) or population(s) is statistically significant with respect to untreated infection(s) or population(s). Curative treatment is also contemplated. Preferably, such control is achieved by increased mortality amongst the pest or pathogen population.

It will be appreciated that control may be via antagonism, which may take a number of forms. In one form, the compositions contemplated herein may simply act as a repellent. In another form, the compositions contemplated herein may render the environment unsuitable or unfavourable for the pest or pathogen. In a further, preferred form, the compositions contemplated herein may incapacitate, render infertile, impede the growth of, impede the spread or distribution of, and/or kill the pest or pathogen. Accordingly, the antagonistic mechanisms include but are not limited to antibiosis, immobilisation, infertility, and toxicity. Therefore, compositions which act as antagonists of one or more pests, such that such compositions are useful in the control of a pest, can be said to have pesticidal activity. For example, compositions that act as antagonists of one or more insects can be said to have insecticidal efficacy. Furthermore, an agent or composition that is or comprises an antagonist of a pest can be said to be a pesticidal agent or a pesticidal composition, for example, an agent that is an antagonist of an insect can be said to be an insecticidal agent. Likewise, a composition that is or comprises an antagonist of an insect can be said to be an insecticidal composition.

Accordingly, as used herein, a “pesticidal composition” is a composition which comprises or includes at least one agent that has pesticidal efficacy.

In various embodiments, said pesticidal efficacy is the ability to repel, incapacitate, render infertile, impede the growth of, or kill one or more pests, including insects or nematodes, for example within 14 days of contact with the pest, such as within 7 days. Particularly contemplated pesticidal efficacy is the ability to kill one or more insect pests of plants within 7 days.

Accordingly, as used herein an “insecticidal composition” is a composition which comprises or includes at least one agent that has insecticidal efficacy.

As used herein the term “culture” refers to a population of microbes, in particular in the context of this disclosure bacteria, together with the media in or on which the population was propagated (i.e. grown) or maintained. For example, the term “whole broth culture” refers to a liquid media and the bacteria therein, for example the population of viable bacteria therein. It will be appreciated that, in certain embodiments contemplated herein, the whole broth culture is one in which substantially all of the bacteria are killed or attenuated, for example, are no longer reproductively viable.

The term “effective amount” as used herein means an amount effective to control or eradicate pests, particularly insect pests.

The term “insecticide” as used herein refers to agents which act to kill or control the growth of insects, including insects at any developmental stage. The related term “insecticidal” will be understood accordingly.

As used herein the term “isolated” means removed from the natural environment in which the subject, typically in this case the B. laterosporus NMI No. V12/001945 bacteria, naturally occurs, such that the subject is separated from some or all of the coexisting materials in the natural system from which the subject has been obtained.

The term “pest” as used herein refers to organisms that are of inconvenience to, or deleterious to, another organism, such as a plant or animal, including a human, whether directly or indirectly. In one embodiment the term refers to organisms that cause damage to animals, including humans, or plants. The damage may relate to plant or animal health, growth, yield, reproduction or viability, and may be cosmetic damage. In certain particularly contemplated embodiments, the damage is of commercial significance. As will be apparent from the context, the term “pest” as used herein will typically refer to one or more organisms that cause damage to plants, for example, cultivated plants, including horticulturally or agriculturally important plants.

The term “plant” as used herein encompasses not only whole plants, but extends to plant parts, cuttings as well as plant products including roots, shoots, leaves, bark, pods, flowers, seeds, stems, callus tissue, nuts and fruit, bulbs, tubers, corms, grains, cuttings, root stock, or scions, and includes any plant material whether pre-planting, during growth, and at or post harvest. Plants that may benefit from the application of the present invention cover a broad range of agricultural and horticultural crops. The compositions described herein are also especially suitable for application in organic production systems.

The term ‘plant derived materials’ refers to products that may be produced from a plant or part thereof. It will be appreciated that a person skilled in the art will know of various examples of plant derived products, such as hay, silage or other types of feed or products.

The term “surroundings” when used in reference to a plant subject to the methods and compositions of the present invention includes water, leaf litter, and/or growth media adjacent to or around the plant or the roots, tubers or the like thereof, adjacent plants, cuttings of said plant, supports, water to be administered to the plant, and coatings including seed coatings. It further includes storage, packaging or processing materials such as protective coatings, boxes and wrappers, and planting, maintenance or harvesting equipment.

Various aspects of the invention are described in further detail in the following subsections. Unless otherwise defined, all technical and scientific terms used herein have the same meaning as is commonly understood by one of ordinary skill in the art to which this invention belongs. In case of conflict, the present specification, including definitions, will control. Although methods and materials similar or equivalent to those described herein can be used in the practice of the invention, examples of suitable methods and materials are described below. The materials, methods, and examples described herein are illustrative only and are not intended to be limiting.

### Polypeptides

As will be appreciated from this disclosure, polypeptides useful in the biological control of insect pests are provided herein. These include full length polypeptides, such as the polypeptides comprising the amino acid sequences depicted in Sequence ID No.s 1, 8, 22, 32, 38, 48, 58, 64, 77, and 87, and functional domains present in those polypeptides, such as those comprising the amino acid sequences presented in, for example, Sequence ID No.s 2 to 7.

The amino acid sequence of a full length S-layer protein from B. laterosporus strain NMI V12/001945 is presented in Sequence ID No. 1. Predicted functional domains identified in this protein, and specifically contemplated for use in the methods and compositions disclosed herein include:
SEQ ID No. 2, a surface layer glycoprotein (SpaA-SLH/G109A) domain, comprising amino acids 89 to 251 of SEQ ID No. 1;
SEQ ID No. 3, an SLH domain (accession pfam00395), comprising amino acids 91 to 124 of SEQ ID No. 1;
SEQ ID No. 4, an SLH domain (accession pfam00395), comprising amino acids 147 to 188 of SEQ ID No. 1;
SEQ ID No. 5, a ComEC domain (accession COG2333), comprising amino acids 208 to 359 of SEQ ID No. 1;
SEQ ID No. 6, a Chitinase (ChiW) domain, comprising amino acids 250 to 322 of SEQ ID No. 1;
SEQ ID No. 7, a Rhoptry domain (accession TIGR01612), comprising amino acids 253 to 509 of SEQ ID No. 1.

The amino acid sequence of a full length adhesion/fimbriae protein from B. laterosporus strain NMI V12/001945 is presented in Sequence ID No. 8. Predicted functional domains identified in this protein, and specifically contemplated for use in the methods and compositions disclosed herein include:
SEQ ID No. 9, a Surface layer protein rSbsC domain, comprising amino acids 17 to 227 of SEQ ID No. 8;
SEQ ID No. 10, a Surface layer protein SbsC domain, comprising amino acids 118 to 279 of SEQ ID No. 8;
SEQ ID No. 11, a S-layer protein sap domain, comprising amino acids 127-219 of SEQ ID No. 8;
SEQ ID No. 12, an SiE domain, comprising amino acids 249 to 419 of SEQ ID No. 8;
SEQ ID No. 13, a PRK12806 superfamily flagellin domain (accession PRK12806), comprising amino acids 360 to 653 of SEQ ID No. 8;
SEQ ID No. 14, a Copper resistance protein C domain, comprising amino acids 432 to 511 of SEQ ID No. 8;
SEQ ID No. 15, an Mfa-like 1 domain (accession pfam13149), comprising amino acids 481 to 636 of SEQ ID No. 8;
SEQ ID No. 16, a Surface layer protein SbsC domain, comprising amino acids 540 to 699 of SEQ ID No. 8;
SEQ ID No. 17, an Hia domain (accession COG5295), comprising amino acids 559 to 812 of SEQ ID No. 8;
SEQ ID No. 18, a ClfB domain, comprising amino acids 597 to 669 of SEQ ID No. 8;
SEQ ID No. 19, a Pfam14262 Carbohydrate-binding domain-containing protein (Cthe 2159) domain (accession pfam14262), comprising amino acids 672 to 798 of SEQ ID No. 8;
SEQ ID No. 20, a DUF4815 domain (accession pfam16075), comprising amino acids 724 to 834 of SEQ ID No. 8;
SEQ ID No. 21, a Flagellar hook-associated protein (FlgK) domain (accession PRK08147), comprising amino acids 733-811 of SEQ ID No. 8.

The amino acid sequence of a full length Efflux pump protein from B. laterosporus strain NMI V12/001945 is presented in Sequence ID No. 22. Predicted functional domains identified in this protein, and specifically contemplated for use in the methods and compositions disclosed herein include:
SEQ ID No. 23, a TolC domain (accession COG1538), comprising amino acids 20 to 391 of SEQ ID No. 22;
SEQ ID No. 24, a TolC domain (accession COG1538), comprising amino acids 32 to 391 of SEQ ID No. 22;
SEQ ID No. 25, a Cation efflux system protein CusC domain, comprising amino acids 60 to 394 of SEQ ID No. 22;
SEQ ID No. 26, an Outer membrane protein TolC domain, comprising amino acids 63 to 394 of SEQ ID No. 22;
SEQ ID No. 27, a Type-I-sec TolC domain (accession TIGR01844), comprising amino acids 63 to 380 of SEQ ID No. 22;
SEQ ID No. 28, a TolC domain (accession PRK09465), comprising amino acids 144 to 264 of SEQ ID No. 22;
SEQ ID No. 29, an SMC_prok_B domain (accession TIGR02168), comprising amino acids 236 to 391 of SEQ ID No. 22;
SEQ ID No. 30, an OEP domain (accession pfam02321), comprising amino acids 290 to 391 of SEQ ID No. 22;
SEQ ID No. 31, an OEP domain (accession pfam02321), comprising amino acids 292 to 369 of SEQ ID No. 22.

The amino acid sequence of a full length S-layer protein from B. laterosporus strain NMI V12/001946 is presented in Sequence ID No. 32. Predicted functional domains identified in this protein, and specifically contemplated for use in the methods and compositions disclosed herein include:
SEQ ID No. 33, a surface layer glycoprotein (SpaA-SLH/G109A) domain, comprising amino acids 74 to 236 of SEQ ID No. 32;
SEQ ID No. 34, an SLH domain (accession pfam00395), comprising amino acids 76 to 109 of SEQ ID No. 32;
SEQ ID No. 35, an SLH domain (accession pfam00395), comprising amino acids 132 to 173 of SEQ ID No. 32;
SEQ ID No. 36, a Tat-secreted protein Rv2525c domain, comprising amino acids 153 to 232 of SEQ ID No. 32;
SEQ ID No. 37, a Chitinase (ChiW) domain, comprising amino acids 235 to 307 of SEQ ID No. 32.

The amino acid sequence of a full length adhesion/fimbriae protein from B. laterosporus strain NMI V12/001946 is presented in Sequence ID No. 38. Predicted functional domains identified in this protein, and specifically contemplated for use in the methods and compositions disclosed herein include:
SEQ ID No. 39, a PRK05035 domain (accession PRK05035), comprising amino acids 911 to 1034 of SEQ ID No. 38;
SEQ ID No. 40, a ATP-synthase Fo_b domain (accession cd06503), comprising amino acids 933 to 1024 of SEQ ID No. 38;
SEQ ID No. 41, a TolC domain (accession COG1538), comprising amino acids 943 to 1131 of SEQ ID No. 38;
SEQ ID No. 42, a Surf_Exclu_PgrA domain (accession TIGR04320), comprising amino acids 944 to 1044 of SEQ ID No. 38;
SEQ ID No. 43, a GARP domain (accession pfam16731), comprising amino acids 955 to 1075 of SEQ ID No. 38;
SEQ ID No. 44, an Invasin IpaB domain, comprising amino acids 972 to 1050 of SEQ ID No. 38;
SEQ ID No. 45, a DUF3584 domain (accession pfam12128), comprising amino acids 982 to 1125 of SEQ ID No. 38;
SEQ ID No. 46, a Hyperosmolarity resistance protein Emb domain, comprising amino acids 1035 to 1124 of SEQ ID No. 38;
SEQ ID No. 47, a Hyalurononglucosaminidase domain, comprising amino acids 1047 to 1130 of SEQ ID No. 38.

The amino acid sequence of a full length Efflux pump protein from B. laterosporus strain NMI V12/001946 is presented in Sequence ID No. 48. Predicted functional domains identified in this protein, and specifically contemplated for use in the methods and compositions disclosed herein include:
SEQ ID No. 49, a TolC domain (accession COG1538), comprising amino acids 23 to 382 of SEQ ID No. 48;
SEQ ID No. 50, a TolC domain (accession COG1538), comprising amino acids 52 to 382 of SEQ ID No. 48;
SEQ ID No. 51, a Cation efflux system protein CusC domain, comprising amino acids 52 to 385 of SEQ ID No. 48;
SEQ ID No. 52, a Type_I_sec domain (accession TIGR01844), comprising amino acids 54 to 371 of SEQ ID No. 48;
SEQ ID No. 53, an Outer membrane protein TolC domain, comprising amino acids 54 to 385 of SEQ ID No. 48;
SEQ ID No. 54, a TolC domain (accession PRK09465), comprising amino acids 135 to 255 of SEQ ID No. 48;
SEQ ID No. 55, an SMC_prok_B domain (accession TIGR02168), comprising amino acids 227 to 382 of SEQ ID No. 48;
SEQ ID No. 56, an OEP domain (accession pfam02321), comprising amino acids 281 to 382 of SEQ ID No. 48;
SEQ ID No. 57, an OEP domain (accession: pfam02321), comprising amino acids 283 to 360 of SEQ ID No. 48.

The amino acid sequence of a full length S-layer protein from B. laterosporus strain NMI V12/001944 is presented in Sequence ID No. 58. Predicted functional domains identified in this protein, and specifically contemplated for use in the methods and compositions disclosed herein include:
SEQ ID No. 59, a surface layer protein (sap) domain, comprising amino acids 89 to 251 of SEQ ID No. 58;
SEQ ID No. 60, an SLH domain (accession pfam00395), comprising amino acids 91 to 124 of SEQ ID No. 58;
SEQ ID No. 61, an SLH domain (accession pfam00395), comprising amino acids 147 to 188 of SEQ ID No. 58;
SEQ ID No. 62, a ComEC domain (accession COG2333), comprising amino acids 208 to 359 of SEQ ID No. 58;
SEQ ID No. 63, a Chitinase (ChiW) domain, comprising amino acids 250 to 322 of SEQ ID No. 58.

The amino acid sequence of a full length adhesion/fimbrae protein from B. laterosporus strain NMI V12/001944 is presented in Sequence ID No. 64. Predicted functional domains identified in this protein, and specifically contemplated for use in the methods and compositions disclosed herein include:
SEQ ID No. 65, a Surface layer protein SbsC domain, comprising amino acids 136 to 236 of SEQ ID No. 64;
SEQ ID No. 66, a Surface layer protein SbsC domain, comprising amino acids 350 to 392 of SEQ ID No. 64;
SEQ ID No. 67, a Surface layer protein rSbsC domain, comprising amino acids 584 to 657 of SEQ ID No. 64;
SEQ ID No. 68, a PRK05035 domain (accession PRK05035), comprising amino acids 1063 to 1186 of SEQ ID No. 64;
SEQ ID No. 69, a ATP-synt_Fo_b domain (accession cd06503), comprising amino acids 1085 to 1176 of SEQ ID No. 64;
SEQ ID No. 70, an TolC domain (accession COG1538), comprising amino acids 1095 to 1283 of SEQ ID No. 64;
SEQ ID No. 71, a Surf_Exclu_PgrA domain (accession TIGR04320), comprising amino acids 1096 to 1196 of SEQ ID No. 64;
SEQ ID No. 72, a GARP domain (accession pfam16731), comprising amino acids 1107 to 1227 of SEQ ID No. 64;
SEQ ID No. 73, a Invasin IpaB domain, comprising amino acids 1123 to 1202 of SEQ ID No. 64;
SEQ ID No. 74, a DUF3584 domain (accession pfam12128), comprising amino acids 1134 to 1277 of SEQ ID No. 64;
SEQ ID No. 75, a Hyperosmolarity resistance protein Emb domain, comprising amino acids 1187 to 1276 of SEQ ID No. 64;
SEQ ID No. 76, a Hypothetical protein ebhA domain, comprising amino acids 1205 to 1281 of SEQ ID No. 64.

The amino acid sequence of a full length Efflux pump protein from B. laterosporus strain NMI V12/001944 is presented in Sequence ID No. 77. Predicted functional domains identified in this protein, and specifically contemplated for use in the methods and compositions disclosed herein include:
SEQ ID No. 78, a TolC domain (accession COG1538), comprising amino acids 23 to 382 of SEQ ID No. 77;
SEQ ID No. 79, a TolC domain (accession COG1538), comprising amino acids 52 to 382 of SEQ ID No. 77;
SEQ ID No. 80, a Cation efflux system protein CusC domain, comprising amino acids 52 to 385 of SEQ ID No. 77;
SEQ ID No. 81, a Type_I_sec domain (accession TIGR01844), comprising amino acids 54 to 371 of SEQ ID No. 77;
SEQ ID No. 82, an Outer membrane protein TolC domain, comprising amino acids 54 to 385 of SEQ ID No. 77;
SEQ ID No. 83, a TolC domain (accession PRK09465), comprising amino acids 135 to 255 of SEQ ID No. 77;
SEQ ID No. 84, an SMC_prok_B domain (accession TIGR02168), comprising amino acids 227 to 382 of SEQ ID No. 77;
SEQ ID No. 85, an OEP domain (accession pfam02321), comprising amino acids 281 to 382 of SEQ ID No. 77;
SEQ ID No. 86, an OEP domain (accession pfam02321), comprising amino acids 283 to 360 of SEQ ID No. 77.

The amino acid sequence of an S-layer protein from B. laterosporus strain NMI V12/001945 comprising 1090 amino acids is presented in Sequence ID No. 87.

The above proteins and polypeptides, including functional domains therefrom and altered or fragmented polypeptides and peptides, such as those produced through proteolytic cleavage, recombinant expression or synthetic production, are examples of the bioactive agents amenable to use according to this disclosure.

In one example, the protein is a protein selected from the group comprising:
a) a polypeptide comprising or consisting of the amino acid sequence depicted in any one of Sequence ID No.s 1 to 87;
b) a polypeptide comprising or consisting of the amino acid sequence corresponding to residues 89 to 322 of Sequence ID No. 1;
c) a polypeptide comprising or consisting of the amino acid sequence corresponding to residues 91 to 322 of Sequence ID No. 1;
d) a polypeptide comprising or consisting of the amino acid sequence corresponding to residues 147 to 322 of Sequence ID No. 1;
e) a polypeptide comprising or consisting of the amino acid sequence corresponding to residues 74 to 307 of Sequence ID No. 32;
f) a polypeptide comprising or consisting of the amino acid sequence corresponding to residues 76 to 307 of Sequence ID No. 32;
g) a polypeptide comprising or consisting of the amino acid sequence corresponding to residues 132 to 307 of Sequence ID No. 32;
h) a polypeptide comprising or consisting of the amino acid sequence corresponding to residues 89 to 322 of Sequence ID No. 58;
i) a polypeptide comprising or consisting of the amino acid sequence corresponding to residues 91 to 322 of Sequence ID No. 58;
j) a polypeptide comprising or consisting of the amino acid sequence corresponding to residues 147 to 322 of Sequence ID No. 58;
k) a polypeptide comprising or consisting of at least 10 contiguous amino acids from any one of a) to j) above;
l) a polypeptide comprising or consisting of at least 20 contiguous amino acids from any one of a) to j) above;
m) a polypeptide comprising or consisting of at least about 10 contiguous amino acids from any one of a) to l) above, wherein said polypeptide has glycoside hydrolase activity;
n) a polypeptide comprising or consisting of at least about 10 contiguous amino acids from any one of a) to m) above, wherein said polypeptide has chitinase activity or one or more chitinase domains;
o) a polypeptide having at least about 90% amino acid identity to any one of a) to n) above;
p) a polypeptide having at least about 95% amino acid identity to any one of a) to n) above;
q) any combination of any two or more of a) to p) above.

In various embodiments, one or more of the polypeptides described above comprises a fusion polypeptide. For example, a fusion polypeptide as contemplated herein will in certain embodiments comprise one or more functional domains derived from, comprising or consisting of one of the sequences presented herein, such as a chitinase domain such as that presented in SEQ ID No. 6 or SEQ ID No. 37, fused to another amino acid sequence to provide a fusion polypeptide.

Those skilled in the art will recognise, on reading this description, that these proteins can be considered representative examples of the pesticidal agents suitable for use as contemplated herein. As such, various uses of and for these polypeptides, particularly in biological control methods such as the control of insect pest populations using bioactive agents of biological origin, for example, are provided.

Proteins suitable for use herein include naturally-occurring proteins and peptides, and derivatives thereof including proteins and peptides having one or more amino acid variations from a naturally-occurring protein or peptide.

The term “amino acid” refers to natural amino acids, non-natural amino acids, and amino acid analogues. Unless otherwise indicated, the term “amino acid” includes both D and L stereoisomers if the respective structure allows such stereoisomeric forms.

Natural amino acids include alanine (Ala or A), arginine (Arg or R), asparagine (Asn or N), aspartic acid (Asp or D), cysteine (Cys or C), glutamine (Gin or Q), glutamic acid (Glu or E), glycine (Gly or G), histidine (His or H), isoleucine (He or I), leucine (Leu or L), Lysine (Lys or K), methionine (Met or M), phenylalanine (Phe or F), proline (Pro or P), serine (Ser or S), threonine (Thr or T), tryptophan (Tip or W), tyrosine (Tyr or Y) and valine (Val or V).

Non-natural amino acids include, but are not limited to, azetidinecarboxylic acid, 2-aminoadipic acid, 3-aminoadipic acid, beta-alanine, naphthylalanine (“naph”), aminopropionic acid, 2-aminobutyric acid, 4-aminobutyric acid, 6-aminocaproic acid, 2-aminoheptanoic acid, 2-aminoisobutyric acid, 3-aminoisbutyric acid, 2-aminopimelic acid, tertiary-butylglycine (“tBuG”), 2,4-diaminoisobutyric acid, desmosine, 2,2′-diaminopimelic acid, 2,3-diaminopropionic acid, N-ethyl glycine, N-ethylasparagine, homoproline (“hPro” or “homoP”), hydroxylysine, allo-hydroxylysine, 3-hydroxyproline (“3Hyp”), 4-hydroxyproline (“4Hyp”), isodesmosine, allo-isoleucine, N-methylalanine (“MeAla” or “Nime”), Nalkylglycine (“NAG”) including N-methylglycine, N-methylisoleucine, N-alkylpentylglycine (“NAPG”) including N-methylpentylglycine. N-methylvaline, naphthylalanine, norvaline (“Norval”), norleucine (“Norleu”), octylglycine (“OctG”), ornithine (“Orn”), pentylglycine (“pG” or “PGly”), pipecolic acid, thioproline (“ThioP” or “tPro”), homoLysine (“hLys”), and homoArginine (“hArg”).

The term “amino acid analogue” refers to a natural or non-natural amino acid where one or more of the C-terminal carboxy group, the N-terminal amino group and side-chain functional group has been chemically blocked, reversibly or irreversibly, or otherwise modified to another functional group. For example, aspartic acid-(beta-methyl ester) is an amino acid analogue of aspartic acid; N-ethylglycine is an amino acid analogue of glycine; or alanine carboxamide is an amino acid analogue of alanine. Other amino acid analogues include methionine sulfoxide, methionine sulfone, S-(carboxymethyl)-cysteine, S-(carboxymethyl) cysteine sulfoxide and S-(carboxymethyl)-cysteine sulfone.

The term “expression construct” refers to a genetic construct that includes elements that permit transcribing the polynucleotide molecule of interest, and, optionally, translating the transcript into a polypeptide. An expression construct typically comprises in a 5′ to 3′ direction:
(1) a promoter, functional in the host cell into which the construct will be introduced,
(2) the polynucleotide to be expressed, and
(3) a terminator functional in the host cell into which the construct will be introduced.

Expression constructs as described herein are inserted into a replicable vector for cloning or for expression, or are incorporated into the host genome.

The term “vector” refers to a polynucleotide molecule, usually double stranded DNA, which is used to transport the genetic construct into a host cell. In certain examples the vector is capable of replication in at least one additional host system, such as E. coli.

A “fragment” of a polypeptide is a subsequence of the polypeptide, typically one that performs a function that is required for activity, such as enzymatic or binding activity, and/or provides a three dimensional structure of the polypeptide or a part thereof, such as an epitope. It will be appreciated that a fragment of a polypeptide may possess or elicit a different function or functions from that possessed or exhibited by the full-length polypeptide from which it is derived.

As used herein, the term “peptide” refers a short polymer of amino acids linked together by peptide bonds. While it will be recognised that the names associated with various classes of amino acid polymers (e.g., peptides, proteins, polypeptides, etc.) are somewhat arbitrary, peptides are generally of about 50 amino acids or less in length. A peptide can comprise natural amino acids, non-natural amino acids, amino acid analogues, and/or modified amino acids. A peptide can be a subsequence of naturally occurring protein or a non-natural, including a synthetic, sequence.

As used herein, the terms “synthetic peptide” and “synthetic polypeptide” encompasses a peptide or a polypeptide produced by synthetic methods, and a peptide or polypeptide having a distinct amino acid sequence from those found in natural peptides and/or proteins. A “synthetic peptide” or “synthetic polypeptide” as used herein can be produced or synthesized by any suitable method (e.g., recombinant expression, chemical synthesis, enzymatic synthesis, etc.), and can include any chemical modification to a parent peptide or polypeptide, and may include, but is not limited to such methods as truncations, deletions, cyclization or non-peptidic synthetic or semi-synthetic derivatives that retain the same biological function(s) as the starting peptide or polypeptide. Methods of protein synthesis, such as solid state synthesis, are well known in the art.

The terms “peptide mimetic” or “peptidomimetic” refer to a peptide-like molecule that emulates a sequence derived from a protein or peptide. A peptide mimetic or peptidomimetic can contain amino acids and/or non-amino acid components. Examples of peptidomimetics include chemically modified peptides, peptoids (side groups are appended to the nitrogen atom of the peptide backbone, rather than to the α-carbons), β-peptides (amino group bonded to the β carbon rather than the α-carbon), etc. Chemical modification includes one or more modifications at amino acid side groups, α-carbon atoms, terminal amine group, or terminal carboxy group. A chemical modification can be adding chemical moieties, creating new bonds, or removing chemical moieties. Modifications at amino acid side groups include, without limitation, acylation of lysine ε-amino groups, N-alkylation of arginine, histidine, or lysine, alkylation of glutamic or aspartic carboxylic acid groups, lactam formation via cyclization of lysine ε-amino groups with glutamic or aspartic acid side group carboxyl groups, hydrocarbon “stapling” (e.g., to stabilize alpha-helix conformations), and deamidation of glutamine or asparagine. Modifications of the terminal amine group include, without limitation, the desamino, N-lower alkyl, N-di-lower alkyl, constrained alkyls (e.g. branched, cyclic, fused, adamantyl) and N-acyl modifications. Modifications of the terminal carboxy group include, without limitation, the amide, lower alkyl amide, constrained alkyls (e.g. branched, cyclic, fused, adamantyl) alkyl, dialkyl amide, and lower alkyl ester modifications. Lower alkyl is C1-C4 alkyl. Furthermore, one or more side groups, or terminal groups, can be protected by protective groups known to the ordinarily skilled peptide chemist. The α-carbon of an amino acid can be mono- or dimethylated.

It will be appreciated that any one of the proteins or peptides described herein in certain embodiments comprises one or more non-naturally occurring amino acids, one or more amino acid analogues, or is or comprises a synthetic peptide or polypeptide or a peptide mimetic. Similarly, it will be appreciated that any one of the proteins or peptides described herein will in certain embodiments be the starting point for one or more modifications, synthetic methods, or protein engineering methods to develop a peptide analogue having a desired biological activity—for example, a qualitatively similar bioactivity as the parent protein or peptide, but an effect of a quantitatively different magnitude, or indeed a different bioactivity from that elicited by the parent protein or peptide.

The term “fusion polypeptide”, as used herein, refers to a polypeptide comprising two or more amino acid sequences, for example two or more polypeptide domains, fused through respective amino and carboxyl residues by a peptide linkage to form a single continuous polypeptide. It should be understood that the two or more amino acid sequences can either be directly fused or indirectly fused through their respective amino and carboxyl termini through a linker or spacer or an additional polypeptide.

The term “polypeptide”, as used herein, encompasses amino acid chains of any length but preferably at least 10 amino acids, including full-length proteins, in which amino acid residues are linked by covalent peptide bonds. Polypeptides described herein are purified natural products, or are produced partially or wholly using recombinant or synthetic techniques. The term may refer to a polypeptide, an aggregate of a polypeptide such as a dimer or other multimer, a fusion polypeptide, a polypeptide variant, or derivative thereof.

It will be understood that, for the particular polypeptides and proteins contemplated herein, natural variations can exist between individual bacterial strains. These variations may be demonstrated by (an) amino acid difference(s) in the overall sequence or by deletions, substitutions, insertions, inversions or additions of (an) amino acid(s) in said sequence. Amino acid substitutions which do not essentially alter biological and immunological activities, are well known. Amino acid replacements between related amino acids or replacements which have occurred frequently in evolution are, inter alia, Ser/Ala, Ser/Gly, Asp/Gly, Asp/Asn, Ile/Val. Other amino add substitutions include Asp/Glu, Thr/Ser, Ala/Gly, Ala/Thr, Ser/Asn, Ala/Val, Thr/Phe, Ala/Pro, Lys/Arg, Leu/Ile, Leu/Val and Ala/Glu. Based on this information, methods for rapid and sensitive protein comparison and determining the functional similarity between homologous proteins were developed. Such amino acid substitutions of the exemplary embodiments described herein, as well as variations having deletions and/or insertions are within the scope of the invention as long as the resulting proteins retain at least a part of one or more of their biological function and/or immunoreactivity. Those variations in the amino acid sequence of a certain protein described herein within the identity ranges and that still provide a protein retaining at least part of one or more functions of the parent protein, or capable of reacting with an antibody specific to the parent protein specifically identified herein are considered as functional equivalents of the proteins identified herein.

When a protein is used, for example for diagnostic or therapeutic purposes or as a biological control agent, for example for reacting with antibodies, or for mediating a biological effect, for example one or more of the biological functions associated with the native protein in vivo, while it can be expedient to do so it is not necessary to use the whole protein. It is also possible to use a polypeptide fragment of that protein (as such or coupled to a carrier or as a component in a fusion polypeptide, for example) or a polypeptide fragment derived from that protein or a related amino acid sequence that is capable of eliciting a desired biological effect, such as an immune response against that protein or of being recognised by an antibody specific to that protein, of mediating a cell-signalling effect, of mediating one or more pesticidal activities, or the like. Such a polypeptide fragment may be referred to with reference to the function it possesses, such as the function it shares with the full-length protein from which it was derived. For example, a polypeptide fragment having an immunological effect may be referred to as an immunogenic fragment, where an “immunogenic fragment” is understood to be a fragment of the full-length protein that retains its capability to induce an immune response in a vertebrate host or be recognised by an antibody specific to the parent protein. Similarly, a polypeptide fragment retaining or possessing one or more biological effects elicited by the full-length protein from which it was derived, or possessing a related or different biological effect, can be referred to herein as a “bioactive fragment” or a “bioactive polypeptide fragment”. Likewise, a polypeptide having a biological effect, such as a polypeptide capable of stimulating a biological response in a cell or eliciting a therapeutic or pesticidal effect, may be referred to herein as a “bioactive fragment” or a “bioactive polypeptide fragment”, or grammatical equivalents thereof.

A variety of techniques is available to identify such polypeptide fragments, as well as DNA fragments encoding such fragments. For example, in the case of immunogenic fragments, such fragments may comprise one or more determinants or epitopes. Well-established empirical and in silico methods for the detection of epitopes exist and are well known to those skilled in the art. For example, computer algorithms are able to designate specific protein fragments as the immunologically important epitopes on the basis of their sequential and/or structural agreement with epitopes that are known. The determination of these regions is typically based on a combination of the hydrophilicity criteria and secondary structural features. An immunogenic fragment (or epitope) usually has a minimal length of 6, more commonly 8 amino acids, preferably more then 8, such as 9, 10, 12, 15 or even 20 or more amino acids. The nucleic acid sequences encoding such a fragment therefore have a length of at least 18, more commonly 24 and preferably 27, 30, 36, 45 or even 60 nucleic acids.

Similarly, those skilled in the art will be aware of methods to identify bioactive fragments using various assays targeted at identifying or detecting a particular biological response. Representative methods suitable for use in the identification or detection of bioactive fragments contemplated herein are presented below, including in the Examples.

The term “variant” with reference to polypeptides encompasses naturally occurring, recombinantly, and synthetically produced polypeptides, including those comprising one or more non-natural amino acids, one or more amino acid analogues, and peptide mimetics. Variant polypeptide sequences preferably exhibit at least 50%, more preferably at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least %, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to a sequences of the present invention. Identity is found over a comparison window of at least 20 amino acid positions, preferably at least 50 amino acid positions, at least 100 amino acid positions, or over the entire length of a polypeptide as described herein.

Polypeptide sequence identity can be determined in the following manner. The subject polypeptide sequence is compared to a candidate polypeptide sequence using BLASTP (from the BLAST suite of programs, version 2.2.10 [October 2004]) in bl2seq, which is publicly available from NCBI (ftp://ftp.ncbi.nih.gov/blast/). The default parameters of bl2seq are utilized except that filtering of low complexity regions should be turned off.

Polypeptide sequence identity may also be calculated over the entire length of the overlap between a candidate and subject polynucleotide sequences using global sequence alignment programs. EMBOSS-needle (available at http:/www.ebi.ac.uk/emboss/align/) and GAP (Huang, X. (1994) On Global Sequence Alignment. Computer Applications in the Biosciences 10, 227-235.) as discussed above are also suitable global sequence alignment programs for calculating polypeptide sequence identity.

Polypeptide variants contemplated herein also encompass those which exhibit a similarity to one or more of the specifically identified sequences that is likely to preserve the functional equivalence of those sequences and which could not reasonably be expected to have occurred by random chance. Such sequence similarity with respect to polypeptides can be determined using the publicly available bl2seq program from the BLAST suite of programs (version 2.2.10 [October 2004]) from NCBI (ftp://ftp.ncbi.nih.gov/blast/). The similarity of polypeptide sequences can be examined using the following unix command line parameters:
bl2seq -i peptideseq1 -j peptideseq2 -F F -p blastp

Variant polypeptide sequences preferably exhibit an E value of less than 1×10⁻¹⁰, more preferably less than 1×10⁻²⁰, less than 1×10⁻³⁰, less than 1×10⁻⁴⁰, less than 1×10⁻⁵⁰, less than 1×10⁻⁶⁰, less than 1×10⁻⁷⁰, less than 1×10⁻⁸⁰, less than 1×10⁻⁹⁰, less than 1×10⁻¹⁰⁰, less than 1×10⁻¹¹⁰, less than 1×10⁻¹²⁰ or less than 1×10⁻¹²³ when compared with any one of the specifically identified sequences.

The parameter -F F turns off filtering of low complexity sections. The parameter -p selects the appropriate algorithm for the pair of sequences. This program finds regions of similarity between the sequences and for each such region reports an “E value” which is the expected number of times one could expect to see such a match by chance in a database of a fixed reference size containing random sequences. For small E values, much less than one, this is approximately the probability of such a random match.

Conservative substitutions of one or several amino acids of a described polypeptide sequence without significantly altering its biological activity are also included in the invention. A skilled artisan will be aware of methods for making phenotypically silent amino acid substitutions (see, e.g., Bowie et al., 1990, Science 247, 1306).

A polypeptide variant contemplated herein also encompasses that which is produced from the nucleic acid encoding a polypeptide, but differs from the wild type polypeptide in that it is processed differently such that it has an altered amino acid sequence. For example, in one embodiment a variant is produced by an alternative splicing pattern of the primary RNA transcript to that which produces a wild type polypeptide.

It will be appreciated that the polypeptides described herein will typically be applied in an agricultural setting in the form of an agricultural composition, formulated to maintain the biological activity of the one or more polypeptides present during storage and application.

### Agricultural Compositions

The compositions for agricultural application, such as in the control of one or more plant pests will typically include at least one agriculturally-acceptable carrier, such as one or more humectants, spreaders, stickers, stabilisers, penetrants, emulsifiers, dispersants, surfactants, buffers, binders, protectants, and other components typically employed in agricultural compositions, or in insecticidal or pesitcidal compositions.

Compositions contemplated herein may be formulated in a variety of different ways without departing from the scope of the present invention. The compositions contemplated herein may be in liquid or solid form. In general the formulation chosen will be dependent on the end application. For example, possible formulations include, but should not be limited to matrixes, soluble powders, granules including water dispersible granules, encapsulations including micro-encapsulations, aqueous solutions, aqueous suspensions, non-aqueous solutions, non-aqueous suspensions, emulsions including microemulsions, pastes, emulsifiable concentrations, and baits.

In various embodiments, the agricultural composition is a liquid composition. Liquid compositions typically include water, saline or oils such as vegetable or mineral oils. Examples of vegetable oils useful in the invention are soy bean oil and coconut oil. The compositions may be in the form of sprays, suspensions, concentrates, foams, drenches, slurries, injectables, gels, dips, pastes and the like. Liquid compositions may be prepared by mixing the liquid agriculturally acceptable carrier with the one or more polypeptides described herein, optionally together with a composition(s) derived from B. laterosporus NMI No. V12/001944, and/or B. laterosporus NMI No. V12/001945, and/or B. laterosporus NMI No. V12/001946, such as a cellular extract or fraction, or a compositions or fractions derived from B. laterosporus NMI No. V12/001944, and/or B. laterosporus NMI No. V12/001945, and/or B. laterosporus NMI No. V12/001946 growth media. Conventional formulation techniques suitable for the production of liquid compositions are well known in the art.

In various embodiments the composition is in solid form. In one example, a solid composition is produced by drying a liquid composition comprising the one or more polypeptides described herein, optionally together with an extract or composition derived from B. laterosporus NMI No. V12/001944, and/or B. laterosporus NMI No. V12/001945, and/or B. laterosporus NMI No. V12/001946. Alternatively, a solid composition useful as described herein is prepared by mixing one or more compositions contemplated herein, for example a proteinaceous composition comprising the one or more polypeptides described herein, optionally together with a composition derived from B. laterosporus NMI No. V12/001944, and/or B. laterosporus NMI No. V12/001945, and/or B. laterosporus NMI No. V12/001946, with a variety of inorganic, organic, and/or biological materials. For example, solid inorganic agricultural carriers suitable for use include carbonates, sulphates, phosphates or silicates, pumice, lime, bentonite, or mixtures thereof. Solid biological materials suitable for use include powdered palm husks, corncob hulls, and nut shells.

Exemplary solid agricultural compositions include those formulated as dusts, granules including water dispersible granules, seed coatings, wettable powders or the like. As is understood in the art, certain solid compositions are applied in solid form, while others are formulated to be admixed with a liquid prior to application, so as to provide a liquid agricultural composition for application.

The compositions contemplated herein are in certain embodiments in the form of controlled release, or sustained release formulations. The compositions contemplated herein in certain embodiments also include other control agents such as pesticides, insecticides, fungicides, nematocides, virucides, growth promoters, nutrients, germination promoters and the like, provided they are compatible with the activity of the composition comprising the one or more polypeptides described herein, and/or other active components that may be present, such as any compositions derived from B. laterosporus NMI No. V12/001944, and/or B. laterosporus NMI No. V12/001945, and/or B. laterosporus NMI No. V12/001946.

In embodiments of particular compositions, for example, of WDG compositions described herein, where viable B. laterosporus NMI No. V12/001944, and/or B. laterosporus NMI No. V12/001945, and/or B. laterosporus NMI No. V12/001946, are present, the same considerations with regard to combinations of components, preparation and application as are discussed above will generally apply.

In certain embodiments, the composition comprises an anti-caking agent, for example, an anti-caking agent selected from talc, silicon dioxide, calcium silicate, or kaelin clay.

In certain embodiments, the composition comprises a wetting agent, such as skimmed milk powder.

In certain embodiments, the composition comprises an emulsifier, such as a soy-based emulsifier such as lecithin, or a vegetable-based emulsifier such as monodiglyceride.

However, other examples of agriculturally acceptable carriers are well known in the art and may be substituted, provided the efficacy of the composition is maintained.

In various embodiments, a desiccation protection agent, such as Deep Fried™, Fortune™, or Fortune Plus™, is admixed to a final concentration of about 1 ml/L prior to application.

In one exemplary embodiment, the composition comprises an oil flowable suspension, such as an oil flowable suspension of one or more polypeptides as described herein.

In a second exemplary embodiment, the composition comprises a wettable powder, dust, pellet, or colloidal concentrate. Such dry forms of the compositions may be formulated to dissolve immediately upon wetting, or alternatively, dissolve in a controlled-release, sustained-release, or other time-dependent manner.

In a third exemplary embodiment, the composition comprises an aqueous solution or suspension of one or more polypeptides as described herein, optionally together with one or more additional agents, for example an extract from B. laterosporus NMI No. V12/001944, and/or B. laterosporus NMI No. V12/001945, and/or B. laterosporus NMI No. V12/001946, as described herein.

Such aqueous solutions or suspensions are in certain embodiments provided as a concentrated stock solution which is diluted prior to application, or alternatively, as a diluted solution ready-to-apply.

In a further exemplary embodiment, the composition comprises a microemulsion.

In various specifically contemplated embodiments, the compositions contemplated herein are formulated as a water dispersible granule (WDG). Water dispersible granule formulations offer advantages over other types of formulations that are agriculturally applied in liquid form. These include simplicity in packaging, ease of handling, and safety. Typically, water dispersible granule formulations are free flowing, low dusting, and readily disperse in water to form either a solution or a homogenous suspension of very small particles suitable for application via conventional techniques and machinery, such as conventional spray equipment and spray nozzles.

The present disclosure provides water dispersible granule formulations comprising the one or more polypeptides described herein. In certain embodiments, the water dispersible granule formulation additionally comprises from about 2% to about 80% (w/w) of a composition derived from B. laterosporus NMI No. V12/001944, and/or B. laterosporus NMI No. V12/001945, and/or B. laterosporus NMI No. V12/001946, such as a cellular extract or a fraction thereof, a culture extract or fraction thereof, or a combination of both. In certain embodiments, viable B. laterosporus NMI No. V12/001944, and/or B. laterosporus NMI No. V12/001945, and/or B. laterosporus NMI No. V12/001946, is present. However, embodiments in which no viable B. laterosporus NMI No. V12/001944, and/or B. laterosporus NMI No. V12/001945, and/or B. laterosporus NMI No. V12/001946, is present are specifically contemplated.

In certain embodiments, the WDG formulation additionally comprises one or more of the following:
a) from about 0% to about 20% (w/w) of one or more surfactants;
b) from about 0% to about 30% (w/w) of one or more binders;
c) from about 0% to about 90% (w/w) of one or more fillers;
d) any combination of a) to c) above, including any combination of two or more of a) to c) above.

In certain embodiments, the WDG formulation additionally comprises water, for example, from about 1% to about 5% (w/w) water, for example, up to about 2% (w/w) water.

In one example, the WDG formulation comprises from about 5% to about 80% (w/w) of bacterial extract or a fraction thereof, and comprises one or more of the following:
a) from about 1% to about 20% (w/w) of one or more surfactants;
b) from about 1% to about 30% (w/w) of one or more binders;
c) from about 1% to about 90% (w/w) of one or more fillers;
d) any combination of a) to c) above, including any combination of two or more of a) to c) above.

In one example, the WDG formulation comprises from about 5% to about 80% (w/w) of bacterial extract or a fraction thereof, and from about 1% to about 20% (w/w) of one or more surfactants; from about 1% to about 30% (w/w) of one or more binders; and from about 1% to about 90% (w/w) of one or more fillers.

In various embodiments, in addition to the one or more polypeptides described herein, the water dispersible granule formulation comprises one or more of the following:
a) from about 2% to about 80% w/w viable B. laterosporus NMI No. V12/001945;
b) from about 2% to about 80% w/w cellular extract obtained from B. laterosporus NMI No. V12/001945;
c) from about 2% to about 80% w/w of a composition comprising or derived from media in which B. laterosporus NMI No. V12/001945 is or has been grown;
d) from about 1% to about 50% w/w one or more wetting agent;
e) from about 1% to about 50% w/w one or more dispersant;
f) from about 2% to about 50% w/w one or more humectant or agent to control water activity;
g) from about 0% to about 50% w/w one or more protectants;
h) from about 0% to about 50% w/w one or more nutrients or mixture thereof;
i) from about 5% to about 80% w/w one or more filler;
j) from about 0% to about 20% w/w one or more antioxidant or UV radiation protectant;
k) from about 1% to about 50% w/w one or more binding agent;
l) from about 0% to about 50% one or more disintegrating agent;
m) from about 0% to about 10% w/w water;
n) any combination of two or more of any of a) to m) above.

In various embodiments, in addition to the one or more polypeptides described herein, the water dispersible granule formulation comprises one or more of the following:
a) from about 2% to about 80% w/w viable B. laterosporus NMI No. V12/001944;
b) from about 2% to about 80% w/w cellular extract obtained from B. laterosporus NMI No. V12/001944;
c) from about 2% to about 80% w/w of a composition comprising or derived from media in which B. laterosporus NMI No. V12/001944 is or has been grown;
d) from about 1% to about 50% w/w one or more wetting agent;
e) from about 1% to about 50% w/w one or more dispersant;
f) from about 2% to about 50% w/w one or more humectant or agent to control water activity;
g) from about 0% to about 50% w/w one or more protectants;
h) from about 0% to about 50% w/w one or more nutrients or mixture thereof;
i) from about 5% to about 80% w/w one or more filler;
j) from about 0% to about 20% w/w one or more antioxidant or UV radiation protectant;
k) from about 1% to about 50% w/w one or more binding agent;
l) from about 0% to about 50% one or more disintegrating agent;
m) from about 0% to about 10% w/w water;
n) any combination of two or more of any of a) to m) above.

In various embodiments, in addition to the one or more polypeptides described herein, the water dispersible granule formulation comprises one or more of the following:
a) from about 2% to about 80% w/w viable B. laterosporus NMI No. V12/001946;
b) from about 2% to about 80% w/w cellular extract obtained from B. laterosporus NMI No. V12/001946;
c) from about 2% to about 80% w/w of a composition comprising or derived from media in which B. laterosporus NMI No. V12/001946 is or has been grown;
d) from about 1% to about 50% w/w one or more wetting agent;
e) from about 1% to about 50% w/w one or more dispersant;
f) from about 2% to about 50% w/w one or more humectant or agent to control water activity;
g) from about 0% to about 50% w/w one or more protectants;
h) from about 0% to about 50% w/w one or more nutrients or mixture thereof;
i) from about 5% to about 80% w/w one or more filler;
j) from about 0% to about 20% w/w one or more antioxidant or UV radiation protectant;
k) from about 1% to about 50% w/w one or more binding agent;
l) from about 0% to about 50% one or more disintegrating agent;
m) from about 0% to about 10% w/w water;
n) any combination of two or more of any of a) to m) above.

In various embodiments, in addition to the one or more polypeptides described herein, the water dispersible granule formulation comprises one or more of the following:
a) from about 2% to about 80% w/w viable B. laterosporus NMI No. V12/001944;
b) from about 2% to about 80% w/w cellular extract obtained from B. laterosporus NMI No. V12/001944;
c) from about 2% to about 80% w/w of a composition comprising or derived from media in which B. laterosporus NMI No. V12/001944 is or has been grown;
d) from about 2% to about 80% w/w viable B. laterosporus NMI No. V12/001945;
e) from about 2% to about 80% w/w cellular extract obtained from B. laterosporus NMI No. V12/001945;
f) from about 2% to about 80% w/w of a composition comprising or derived from media in which B. laterosporus NMI No. V12/001945 is or has been grown;
g) from about 2% to about 80% w/w viable B. laterosporus NMI No. V12/001946;
h) from about 2% to about 80% w/w cellular extract obtained from B. laterosporus NMI No. V12/001946;
i) from about 2% to about 80% w/w of a composition comprising or derived from media in which B. laterosporus NMI No. V12/001946 is or has been grown;
j) from about 1% to about 50% w/w one or more wetting agent;
k) from about 1% to about 50% w/w one or more dispersant;
l) from about 2% to about 50% w/w one or more humectant or agent to control water activity;
m) from about 0% to about 50% w/w one or more protectants;
n) from about 0% to about 50% w/w one or more nutrients or mixture thereof;
o) from about 5% to about 80% w/w one or more filler;
p) from about 0% to about 20% w/w one or more antioxidant or UV radiation protectant;
q) from about 1% to about 50% w/w one or more binding agent;
r) from about 0% to about 50% one or more disintegrating agent;
s) from about 0% to about 10% w/w water;
t) any combination of two or more of any of a) to s) above.

In various embodiments, the wetting agent or dispersant is selected from the group comprising Sodium lignosulphonate, Sodium methoxy-lignosulphonate, Sodium polycarboxylate, Potassium polycarboxylate, Phosphate ester surfactants, including ethoxylated alcohol ether phosphate esters, Sodium aryl sulphonates, Ethoxylated linear alcohols, alkyl phenol alcohols, Alkyl polyglucoside, Alkali salts of dioctyl sulphosuccinate, including sodium dioctyl sulphosuccinate, and any combination of any two or more thereof.

In various embodiments, the filler is selected from the group comprising Kaolin, Talc, Bentonite, Atapulgite, Sepiolite, Vermiculite, Silica, including ground silica, fumed silica, and precipitated silica, Perlite, Cellulosic fibre, such as ground nut shells, husks, and the like, and any combination of any two or more thereof.

In various embodiments, the binding agent is selected from the group comprising Sugars, such as sucrose, fructose, maltodextrin, and the like, Acrylic or maleic acid polymers or copolymers, Polyvinylpyrrolidone, Starch and modified starch, Cellulosic gums, such as CMC, HEC, HMC, Polysaccharide gums, such as guar, Xanthan, pullulan, carrageenan, gellan, agar, alginate, chitin and chitosan, and the like, and any combination of any two or more thereof.

In various embodiments, the protectant is selected from the group comprising antioxidants, UV protectants, preservatives, antidessicants, and emollients, and any combination of any two or more thereof.

In various embodiments, the antioxidant is selected from the group comprising water soluble antioxidants, oil soluble antioxidants, including antioxidants such as ascorbic acid and salts thereof, such as sodium ascorbate, calcium ascorbate, etc., vitamin E and other phenolic antioxidants, TBHQ, Propyl gallate and other gallic acid esters, tert-butylhydroquinone (TBHQ), and any combination of any two or more thereof.

In various embodiments, the emollient is selected from the group comprising vegetable oils, waxes, or greases, mineral oils, waxes or greases, mono and diglycerides of longer chain fatty acids, and any combination of any two or more thereof.

In various embodiments, the humectant of agent to control water activity is selected from the group comprising one or more sugars, such as glucose, glycerol, propylene glycol, betaine, one or more salts that can serve to limit water activity, and any combination of any two or more thereof.

In particular embodiments, WDG formulations contemplated herein, for example those prepared via wet granulation processes, do not require a disintegrant. The present disclosure also relates to liquid formulations comprising water dispersible granule formulations dispersed in water, processes for the preparation of water dispersible granule formulations using wet granulation processes, and methods of administering an effective amount of water dispersible granule formulations to a plant or its surroundings, for example to control one or more insect pests.

One suitable method for preparing WDG formulations is a direct granulation method, in which a composition comprising the one or more polypeptides described herein is directly applied to the dry ingredients to form an extrudable paste. The paste is then formed into an elongate extrudate. In one embodiment, the extrudate is dried, and may then be cut or granulated when dry, while in another embodiment the extrudate is agitated or cut to form granules in a granulating mixer before being dried. Typically, the damp granules are dried in a fluid bed drier to achieve the desired moisture content.

It will be appreciated that the moisture content can vary depending on the uses to which the WDG is to be put, the storage expectations for the WDG product, or whether viable cells or spores are present in the final product or not.

It will be appreciated that this method advantageously employs a single drying step to produce the final product.

Another suitable method for preparing WDG formulations is an indirect granulation method in which the composition comprising the one or more polypeptides described herein is first dried to the desired moisture content/non-volatile material content before addition to the other WDG ingredients. Additional water is normally required to provide enough moisture to form an extrudable paste and this in turn has to be dried off in the final drying process.

The initial drying of the protein-containing composition can be achieved by any suitable drying method, such as batch drying, vacuum falling film evaporating, spray drying or freeze drying. In formulations in which viable cells or spores are to be present, freeze drying and vacuum spray drying will typically be used, as the gentle conditions achievable with these methods help maximise viability.

This method has the advantage of reducing the water activity of the product to a low level that improves the stability until it is ready for incorporation into WD granules and can also be used to increase the level of active material in the final granule.

A further suitable method for preparing WDG formulations is the so-called ‘Sorbie’ process in which absorbent dispersible granules are produced using inert materials and a binder in the absence of the active agent(s). Subsequently, the composition comprising the one or more polypeptides described herein is sprayed onto the absorbent granules, typically while the absorbent granules are fluidized, for example, in a fluid bed drier, followed by gentle heating to dry the granules.

This method has the advantage of allowing very gentle final drying conditions, for example, for formulations comprising heat-sensitive ingredients, such as the one or more polypeptides described herein, or viable cells or spores, while more aggressive conditions can be used to produce the inert ‘sorbie’ particles. This allows a degree of flexibility in process control, in which bulk ‘sorbie’ particles can be produced independently of the active agent(s) composition(s). It will be appreciated that the production of active agent(s), such as the production of the composition comprising the one or more polypeptides described herein, will often be the rate-limiting step, such that shorter production times can be achieved after active agent(s) production is completed.

In certain embodiments relating to water dispersible granule formulations, the preparation of water dispersible granules comprising the one or more polypeptides described herein and/or a composition as contemplated herein via wet granulation enables the efficient preparation and recovery of granules of regular size and shape, and thus of similar dissolution and handling characteristics, among other advantages. Such regularity in particle size can be problematic to achieve with other formulation methods, such as dry compaction and fragmentation, which typically produces chips of irregular size and shape. In certain embodiments, the combination of wet granulation and lack of disintegrants in representative examples of WDG formulations provides an efficient and effective formulation for agricultural application and pest control.

In certain embodiments, the compositions described herein may be used in conjunction with other treatments such as cryoprotectants, surfactants, detergents, soaps, dormant oils, polymers, and/or time-release or biodegradable carrier formulations that permit long-term dosing of a target area following a single application of the formulation.

The compositions as described herein may also be used in consecutive or simultaneous application to a plant population or an environmental site singly or in combination with one or more additional agents, such as insecticides, pesticides, chemicals, fertilizers, or other compounds.

As discussed herein, compositions as described herein may be formulated as, for example, concentrates, solutions, sprays, aerosols, immersion baths, dips, emulsions, wettable powders, soluble powders, suspension concentrates, dusts, granules, water dispersible granules, microcapsules, pastes, gels and other formulation types by well-established procedures.

These procedures will frequently include mixing and/or milling of the active components with agriculturally acceptable carrier substances, such as fillers, solvents, excipients, surfactants, suspending agents, spreaders/stickers (adhesives), antifoaming agents, dispersants, wetting agents, drift reducing agents, auxiliaries and adjuvants.

In one embodiment solid carriers include but are not limited to mineral earths such as silicic acids, silica gels, silicates, talc, kaolin, attapulgus clay, limestone, lime, chalk, bole, loess, clay, dolomite, diatomaceous earth, aluminas calcium sulfate, magnesium sulfate, magnesium oxide, ground plastics, fertilizers such as ammonium sulfate, ammonium phosphate, ammonium nitrate, and ureas, and vegetable products such as grain meals, bark meal, wood meal, and nutshell meal, cellulosic powders and the like.

As solid carriers for granules, including for example the WDG formulations specifically contemplated herein, the following are suitable: crushed or fractionated natural rocks such as calcite, marble, pumice, sepiolite and dolomite; synthetic granules of inorganic or organic meals; granules of organic material such as sawdust, coconut shells, corn cobs, corn husks or tobacco stalks; kieselguhr, tricalcium phosphate, powdered cork, or absorbent carbon black; water soluble polymers, resins, waxes; or solid fertilizers. Such solid compositions may, if desired, contain one or more compatible wetting, dispersing, emulsifying or colouring agents which, when solid, may also serve as a diluent.

In various embodiments the carrier may also be liquid, for example, water; alcohols, particularly butanol or glycol, as well as their ethers or esters, particularly methylglycol acetate; ketones, particularly acetone, cyclohexanone, methylethyl ketone, methylisobutylketone, or isophorone; petroleum fractions such as paraffinic or aromatic hydrocarbons, particularly xylenes or alkyl naphthalenes; mineral or vegetable oils; aliphatic chlorinated hydrocarbons, particularly trichloroethane or methylene′ chloride; aromatic chlorinated hydrocarbons, particularly chlorobenzenes; water-soluble or strongly polar solvents such as dimethylformamide, dimethyl sulfoxide, or N-methylpyrrolidone; liquefied gases; or the like or a mixture thereof.

In one embodiment surfactants include nonionic surfactants, anionic surfactants, cationic surfactants and/or amphoteric surfactants and promote the ability of aggregates to remain in solution during spraying.

Spreaders/stickers promote the ability of the compositions as described herein to adhere to plant surfaces. Examples of surfactants, spreaders/stickers include but are not limited to Tween and Triton (Rhom and Hass Company), Deep Fried™, Fortune®, Pulse, C. Daxoil®, Codacide Oil®, D-C. Tate®, Supamet Oil, Bond®, Penetrant, Glowelt® and Freeway, Citowett®, Fortune Plus™, Fortune Plus Lite, Fruimec, Fruimec lite, alkali metal, alkaline earth metal and ammonium salts of aromatic sulfonic acids, e.g., ligninsulfonic acid, phenolsulfonic acid, naphthalenesulfonic acid and dibutylnaphthalenesulfonic acid, and of fatty acids, alkyl and alkylaryl sulfonates, and alkyl, lauryl ether and fatty alcohol sulfates, and salts of sulfated hexadecanols, heptadecanols, and octadecanols, salts of fatty alcohol glycol ethers, condensation products of sulfonated naphthalene and naphthalene derivatives with formaldehyde, condensation products of naphthalene or naphthalenesulfonic acids with phenol and formaldehyde, polyoxyethylene octylphenol ethers, ethoxylated isooctylphenol, ethoxylated octylphenol and ethoxylated nonylphenol, alkylphenol polyglycol ethers, tributylphenyl polyglycol ethers, alkylaryl polyether alcohols, isotridecyl alcohol, fatty alcohol ethylene oxide condensates, ethoxylated castor oil, polyoxyethylene alkyl ethers, ethoxylated polyoxypropylene, lauryl alcohol polyglycol ether acetal, sorbitol esters, lignin-sulfite waste liquors and methyl cellulose. Where selected for inclusion, one or more agricultural surfactants, such as Tween are desirably included in the composition according to known protocols.

Wetting agents reduce surface tension of water in the composition and thus increase the surface area over which a given amount of the composition may be applied. Examples of wetting agents include but are not limited to salts of polyacrylic acids, salts of lignosulfonic acids, salts of phenolsulfonic or naphthalenesulfonic acids, polycondensates of ethylene oxide with fatty alcohols or fatty acids or fatty esters or fatty amines, substituted phenols (particularly alkylphenols or arylphenols), salts of sulfosuccinic acid esters, taurine derivatives (particularly alkyltaurates), phosphoric esters of alcohols or of polycondensates of ethylene oxide with phenols, esters of fatty acids with polyols, or sulfate, sulfonate or phosphate functional derivatives of the above compounds.

In one embodiment the preferred method of applying the composition as described herein is to spray a dilute or concentrated solution by handgun or commercial airblast.

As described above, the compositions as described herein may be used alone or in combination with one or more other agricultural agents, including pesticides, insecticides, acaracides, fungicides or bactericides (provided such fungicides or bactericides are not detrimental or toxic to any fungi or bacteria that are present in the composition), herbicides, antibiotics, antimicrobials, nemacides, rodenticides, entomopathogens, pheromones, attractants, plant growth regulators, plant hormones, insect growth regulators, chemosterilants, microbial pest control agents, repellents, viruses, phagostimulents, plant nutrients, plant fertilisers and biological controls. When used in combination with other agricultural agents the administration of the two or more agents or formulations may be separate, simultaneous or sequential. Specific examples of these agricultural agents are known to those skilled in the art, and many are readily commercially available.

Examples of plant nutrients include but are not limited to nitrogen, magnesium, calcium, boron, potassium, copper, iron, phosphorus, manganese, molybdenum, cobalt, boron, copper, silicon, selenium, nickel, aluminum, chromium and zinc.

Examples of antibiotics include but are not limited to oxytetracyline and streptomycin.

Examples of fungicides include but are not limited to the following classes of fungicides: carboxamides, benzimidazoles, triazoles, hydroxypyridines, dicarboxamides, phenylamides, thiadiazoles, carbamates, cyano-oximes, cinnamic acid derivatives, morpholines, imidazoles, beta-methoxy acrylates and pyridines/pyrimidines.

Further examples of fungicides include but are not limited to natural fungicides, organic fungicides, sulphur-based fungicides, copper/calcium fungicides and elicitors of plant host defences.

Examples of natural fungicides include but are not limited to whole milk, whey, fatty acids or esterified fatty acids.

Examples of organic fungicides include but are not limited to any fungicide which passes an organic certification standard such as biocontrol agents, natural products, elicitors (some of may also be classed as natural products), and sulphur and copper fungicides (usually limited to restricted use). An example of a sulphur-based fungicide is Kumulus™ DF (BASF, Germany). An example of a copper fungicide is Kocide® 2000 DF (Griffin Corporation, USA).

Examples of elicitors include but are not limited to chitosan, Bion™, BAB A (DL-3-amino-n-butanoic acid, β-aminobutyric acid) and Milsana™ (Western Farm Service, Inc., USA).

In some embodiments non-organic fungicides may be employed. Examples of nonorganic fungicides include but are not limited to Bravo™ (for control of PM on cucurbits); Supershield™ (Yates, NZ) (for control of Botrytis and PM on roses); Topas® 200EW (for control of PM on grapes and cucurbits); Flint™ (for control of PM on apples and cucurbits); Amistar® WG (for control of rust and PM on cereals); and Captan™, Dithane™, Euparen™, Rovral™, Scala™, Shirlan™, Switch™ and Teldor™ (for control of Botrytis on grapes).

Examples of pesticides include but are not limited to azoxystrobin, bitertanol, carboxin, Cu₂O, cymoxanil, cyproconazole, cyprodinil, dichlofluamid, difenoconazole, diniconazole, epoxiconazole, fenpiclonil, fludioxonil, fluquiconazole, flusilazole, flutriafol, furalaxyl, guazatin, hexaconazole, hymexazol, imazalil, imibenconazole, ipconazole, kresoxim-methyl, mancozeb, metalaxyl, R-metalaxyl, metconazole, oxadixyl, pefurazoate, penconazole, pencycuron, prochloraz, propiconazole, pyroquilone, SSF-109, spiroxamin, tebuconazole, thiabendazole, tolifluamid, triazoxide, triadimefon, triadimenol, triflumizole, triticonazole and uniconazole.

An example of a biological control agent is the BotryZen™ biological control agent comprising Ulocladium oudemansii.

The compositions may also comprise a broad range of additives such as stablisers and penetrants used to enhance the activity of the composition, and so-called ‘stressing’ additives such as potassium chloride, glycerol, sodium chloride and glucose. Additives may also include compositions which assist in maintaining stability or, when one or more microbes are present in the composition, microorganism viability, for example, during long term storage, for example unrefined corn oil and so called invert emulsions.

As will be appreciated by those skilled in the art, it is important that any additives used are present in amounts that do not interfere with the effectiveness of the composition.

### Application

In one example, compositions as described herein are applied directly to the plant or its surroundings. In one embodiment, a composition as contemplated herein is applied to the environment of the pest, typically on to plants to be protected, equipment, ground or air. For example, a composition as described herein is admixed with a solvent, for example water, and applied as described herein.

In one embodiment, a composition as described herein is applied directly to the pest. for example, by spraying, dipping, dusting or the like. It will be appreciated that, in certain circumstances, application to a plant or its surroundings will have the potential to include at least some direct application to a pest, for example, a pest already present on the plant or its surroundings.

In one embodiment, for example of a method for controlling one or more plant pests, the method comprising applying to a plant or its surroundings a composition as described herein.

The concentration of composition, or of active component(s) comprising the compostion, for example the one or more polypeptides described herein, which is used for environmental, systemic, topical, or foliar application will vary widely depending upon the nature of the particular formulation, means of application, environmental conditions, and degree of biocidal activity.

In certain embodiments, a typical application rate of active agent, for example of the one or more polypeptides described herein, is from about 0.1 g/hectare to 10,000 g/hectare. Commonly, the application rate is from about 10 g/hectare to 5,000 g/hectare, or 50 to 1500 g/hectare.

In various embodiments, the composition is admixed with water to a final concentration of active agent, for example the one or more polypeptides described herein, of about 0.5 gm/L to about 10 gm/L prior to application, for example to a final concentration of about 5 gm/L.

The composition will in various embodiments be administered to a particular plant or target area in one or more applications as needed, with a field application rate per hectare ranging on the order of from about 50 g/hectare to about 500 g/hectare of active ingredient, or alternatively, from about 500 g/hectare to about 1000 g/hectare may be utilized. In certain instances, it may even be desirable to apply the formulation to a target area at an application rate of from about 1000 g hectare to about 5000 g hectare or more of active component, for example of the one or more polypeptides described herein. In fact, all application rates in the range of from about 0.1 g of active agent per hectare to about 10,000 g/hectare are contemplated to be useful in the management, control, or killing, of target organisms using such formulations. As such, rates of about 100 g/hectare, about 200 g/hectare, about 300 g/hectare, about 400 g hectare, about 500 g/hectare, about 600 g/hectare, about 700 g/hectare, about 800 g/hectare, about 900 g/hectare, about 1 kg/hectare, about 1.1 kg/hectare, about 1.2 kg/hectare, about 1.3 kg/hectare, about 1.4 kg/hectare, about 1.5 kg/hectare, about 1.6 kg/hectare, about 1.7 kg/hectare, about 1.8 kg/hectare, about 1.9 kg/hectare, about 2.0 kg/hectare, about 2.5 kg/hectare, about 3.0 kg/hectare, about 3.5 kg/hectare, about 4.0 kg/hectare, about 4.5 kg/hectare, about 6.0 kg/hectare, about 7.0 kg/hectare, about 8.0 kg/hectare, about 8.5 kg/hectare, about 9.0 kg/hectare, and even up to and including about 10.0 kg/hectare or greater of active component may be utilized in certain agricultural, industrial, and domestic applications of the formulations described hereinabove.

Convenient and effective rates of application can be achieved by formulating the composition to deliver an effective amount of the one or more polypeptides described herein, and applying said composition at a rate of about 1 L to 100 L per hectare. As discussed herein, such an application rate can be conveniently achieved by dissolution of the composition in a larger volume of agriculturally acceptable solvent, for example, water.

In various embodiments, the composition is admixed with water prior to application. In one embodiment, the composition is admixed with water and applied in at least about 100 L water/Ha, in at least about 150 L/Ha, in at least about 200 L/Ha, in at least about 250 I/Ha, in at least about 300 L/Ha, in at least about 350 L Ha, in at least about 400 L/Ha, in at least about 450 L/Ha, or in at least about 500 L/Ha.

Spraying, dusting, soil soaking, seed coating, foliar spraying, misting, aerosolizing and fumigation are all possible application techniques.

Generally, said application is by spraying.

Compositions formulated for other methods of application such as injection, rubbing or brushing, may also be used, as indeed may any known art method. Indirect applications of the composition to the plant surroundings or environment such as soil, water, or as seed coatings are possible.

As discussed above, the concentration at which the compositions are to be applied so as to be effective control compositions may vary depending on the end use, physiological condition of the plant; type (including plant species) or number of plants to be controlled; temperature, season, humidity, stage in the growing season and the age of plant; number and type of conventional treatments (including herbicides) being applied; and plant treatments (such as leaf plucking and pruning).

Other application techniques, including dusting, sprinkling, soil soaking, soil injection, seed coating, seedling coating, aerating, misting, atomizing, fumigating, aerosolizing, and the like, are also feasible and may be required under certain circumstances. These application procedures are also well-known to those of skill in the art.

Applications may be once only or repeated as required. Application at different times in plant life cycles, are also contemplated. For example, at harvest to prevent or minimise post harvest attack by pests.

Young seedlings are typically most susceptible to damage from competing plants and pests, such as insect pests. Therefore, application of the compositions as described herein to freshly planted-out crops, prior to emergence, is contemplated, as is application on emergence.

Repeated applications at the same or different times in a crop cycle are also contemplated. The compositions as described herein may be applied either earlier or later in the season. This may be over flowering or during fruiting, or immediately prior to harvest of the desired crop or plant, or after harvest to protect necrotic or senescing leaves, fruit, stems, machine harvested stalks and the like.

Application may be at a time before or after bud burst and before and after harvest. However, treatment preferably occurs between flowering and harvest. To increase efficacy, multiple applications (for example, 2 to 6 applications over the stages of flowering through fruiting) of the compositions as described herein is contemplated.

The compositions as described herein may also be formulated for preventative or prophylactic application to an area, and may in certain circumstances be applied to and around farm equipment, barns, domiciles, or agricultural or industrial facilities, and the like.

The compositions and methods described herein are applicable to any plant or its surroundings. Such plants include cereal, vegetable and arable crops, grasses, lawns, pastures, fruit trees and ornamental trees and plants.

Arable crops which may particularly benefit from use of the compositions and strain(s) as described herein include crucifers and brassicas. For example, cabbage, broccoli, cauliflower, brussel sprouts and bok choy.

Exemplary plants are in certain embodiments monocotyledonous or dicotyledonous plants such as alfalfa, barley, canola, corn, cotton, flax, kapok, peanut, potato, oat, rice, rye, sorghum, soybean, sugarbeet, sugarcane, sunflower, tobacco, tomato, wheat, turf grass, pasture grass, berry, fruit, legume, vegetable, ornamental plants, shrubs, cactuses, succulents, and trees. In further illustrative embodiments, the plant may be any plant, including plants selected from the order Solanales, including plants from the following families: Convolvulaceae, Hydroleaceae, Montiniaceae, Solanaceae, and Sphenocleaceae, and plants from the order Asparagales, including plants from the following families: Amaryllidaceae, Asparagaceae, Asteliaceae, Blandfordiaceae, Boryaceae, Doryanthaceae, Hypoxidaceae, Iridaceae, Ixioliriaceae, Lanariaceae, Orchidaceae, Tecophilaeaceae, Xanthorrhoeaceae, and Xeronemataceae.

The invention is further described with reference to the following examples. It will be appreciated that the invention as claimed is not intended to be limited in any way by these examples.

### EXAMPLES

### Example 1: Cellular Location of Insecticidal Activity

This example describes an assessment of the cellular localisation of the insecticidal activity from Brevibacillus laterosporus strain NMI No. V12/001945 (also referred to herein as Bl45).

### Methods

The method used in this and certain experiments presented in subsequent examples were as follows:

### Gradient Centrifugation of Bl45

A volume of 20 ml of a 6-day-sporulated culture, cultured in mLB⁺ medium as described above, was harvested by centrifugation at 15000×g for 15 minutes at 4° C. The spore pellet was resuspended in 1.25 ml solution E (10 mM Tris-HCl, 150 mM NaCl, 1 mM EDTA and 0.2% Triton X-100; pH 7.6), containing protease inhibitors (cOmplete™, Mini, EDTA-free protease inhibitor cocktail). The spore suspension was sonicated twice for 30 seconds on ice at 7 amplitude microns, with a 60 s pause in between sonications. Five-hundred μl sonicated sample was added on top of a discontinuous gradient of 20%, 30%, 40% and 50% iodixanol each of 0.75 ml. The discontinuous iodixanol gradient was prepared from a 60% commercial stock solution (OptiPrep™, Axis-Shield). A total of 6 tubes containing the discontinuous gradient and 500 μl sample were centrifuged under vacuum at 160,000×g in an ultracentrifuge (Beckman Coulter Optima™, L-100K ultracentrifuge) using a swing bucket rotor (SW55Ti) for 2 hours at 4° C. Gradient bands were harvested and washed twice in Milli Q water (MQW) by centrifugation at 10,000×g for 10 minutes at 4° C. Washed pellets were resuspended in storage buffer (50 mM Tris-HCl; pH 7.8) and stored at 4° C. until further use. A DBM bioassay was set up with gradient fractions of interest as described below. Sterile MQW was used as a negative control.

### General Bioassay Setup

Cabbage discs were cut out from a green cabbage leaf using a core borer with a 3 cm diameter. The leaf discs were washed in dH₂O prior to treatment application. 100 μl of treatment was spread onto both sides of each cabbage leaf disc and left to air dry on an angle in a sterile petri dish in a class 1 laminar flow cabinet. The air-dried leaf discs were put into sterile plastic containers (HuhTamaki, 30 ml volume) containing 3 cm diameter filter papers (Labserv, qualitative paper). The filter papers were hydrated with a 100 μl sterile MQW before the leaf discs were added to the containers to prevent the cabbage discs from drying out too rapidly. Six in-house colony reared second to third instar DBM, Plutella xylostella, caterpillars were added to each leaf disc with 3 blocks per treatment. The number of caterpillars per treatment was therefore 18 (N=18). Bioassays were set up according to a randomised block design. DipelDF Bacillus thuringiensis subsp. kurstaki H-3a, 3b HDI (Nufarm, Valent BioSciences® Corporation) was used as a positive control at 32000 units/ml (1 mg/ml). Sterile MQW or mLB⁺ medium were used as negative controls. All treatments, including the controls, contained 0.5% Synoil adjuvant surfactant (Orion Agriscience). The bioassays were incubated at 23-25° C. with a 16:8 hour light dark cycle. Mortality rates were recorded every 24 hours after incubation for 4-9 days. The bioassay results were analysed by a general ANOVA using Genstat version 16. Treatments with the constant values 0 or 100 were not included into the ANOVA to maintain variability in the statistical analysis. The least significant effect (LSE) was used to compare a constant valued treatment with a non-constant valued treatment.

### Size Exclusion Chromatography of Bl45 Culture Supernatant Ammonium Sulphate Precipitate

### Ammonium Sulphate Precipitation

A 100 ml of culture supernatant filtered through a 0.2 μm filter (Millpore) was collected from a six-day-old sporulated culture of Bl45. A magnetic stirrer was used to stir the culture supernatant slowly on ice while ammonium sulphate was gradually added until it reached a concentration of 85% w/v, causing the proteins to precipitate. The precipitate was collected by centrifugation at 10,000×g for 20 minutes at 4° C. The pellet was resuspended in 20 ml resuspension buffer (20 mM Tris-HCl and 150 mM NaCl; pH 7.5). Subsequently, the suspension was washed three times in resuspension buffer by centrifugation at 8000×g for 15 minutes at 4° C., using a Vivaspin 20, 5000 molecular weight cut off (MWCO) concentrator column (GE Healthcare). After the third wash, the concentrate was concentrated down to 5 ml using the Vivaspin 20, 5000 MWCO column, and stored at 4° C. until further use.

### Size Exclusion Chromatography

The 5 ml desalted protein suspension, as described above, was concentrated to 1 ml by centrifugation at 5800×g for 10 minutes at 15° C. using a 10,000 MWCO concentrator column (Millipore). The concentrate was resuspended in 5 ml TBS column buffer (25 mM Tris-HCl and 150 mM NaCl; pH 7.4) and concentrated down to 1 ml using the 10,000 MWCO concentrator column. The sample was subsequently injected into a Sephacryl S200 High Resolution (GE Health care Life Sciences) column (1.5×42 cm) for the separation of the proteins present in the sample. The size exclusion chromatography ran for 2.5 hours at a speed of 1 ml/minute operated by a Bio-Rad Biologic LP (low Pressure) chromatography system. Fractions of 1 ml were collected by a fraction collector (BioFrac™, BioRad) and stored at 4° C. until further use. Fractions were analysed by SDS-PAGE, Native PAGE and Bradford measurements according to standard protocols. A DBM bioassay was conducted with the protein fractions of interest as described above. Sterile MQW was used as a negative control.

### The Collection of Crude Culture Fractions of Bl45

A volume of 50 ml of a six day old sporulated culture of Bl45, cultured in mLB⁺, was centrifuged at 10,000×g for 15 minutes at 4° C. to separate the spores from the culture supernatant. Prior to centrifugation, 5-10 ml of full strength culture was kept in storage at 4° C. or −20° C. to be tested in DBM insect bioassays. The spore pellet was washed thrice in 50 ml of sterile MQW by centrifugation at 10,000×g for 15 minutes at 4° C. The washed spores were resuspended in 50 ml sterile MQW and stored at 4° C. or −20° C. until further use. The culture supernatant was centrifuged six-seven times under the same conditions as above to remove as many particles as possible. Subsequently, the culture supernatant was filtered through a 0.8 μm/0.2 μm vacuum filter. The filtered culture supernatant was kept at 4° C. or −20° C. until further use. A part of the culture supernatant was heated at 65° C. and/or 95° C. in a water bath prior to being tested for toxicity against DBM caterpillars. The samples were bioassayed against DBM as described above. Bioassay treatments consisted of the full strength culture, the washed spore suspension, the culture supernatant kept at 4° C., heated culture supernatant at 65° C. and/or heated at 95° C. Sterile MQW was used as a negative control.

### Reversed Phase High Pressure Liquid Chromatography (RP-HPLC) of Bl45 Culture Supernatant

### Chemicals/Buffers

HPLC mobile phase: Buffer A: 50 mM KH₂PO₄; pH 2.5, 0.45 μm Nylon membrane filtered.

Solution B: 100% methanol (gradient HPLC grade).

Blank: mLB⁺ medium (7.7 mM K₂HPO₄, 42 mM KH₂PO₄, 2.5% w/v LB, 0.0125% w/v NaOH, 5.25 mM NTA, 0.59 mM MgSO₄, 0.91 mM CaCl₂, 0.04 mM FeSO₄, 2.5 mM MnCl₂ and 1% w/v glucose; pH 7.6).

### HPLC Equipment and Software

The Agilent 1100 Series HPLC system (Agilent Technologies) was used for separation and fraction collection. The equipment included a Quaternary pump, a vacuum degasser, an autosampler with a 100 μl injection loop, an autosampler thermostat and a Diode Array Detector (DAD), including an Agilent 1260 Infinity Fraction Collector. Software used to control the HPLC-equipment was Agilent ChemStation 32.

### HPLC Conditions

The HPLC conditions were adapted from Gohar and Perchat (2001). A prodigy HPLC C₁₈ RP analytical column, 5 μm 250 mm×4.6 mm (Phenomenex, USA) was used for separation. A 100 μl of 0.2 μm filtered Bl45 culture supernatant, harvested from a 6-day-old sporulating culture as described above was injected. The HPLC pump flow rate was 0.5 ml/min, the linear gradient was from 5% to 15% Solution B developed over 20 minutes. UV-absorption was monitored at 260 nm.

### Sample Preparation Prior to Bioassay Setup

A total of ten 100 μl sample injections were conducted. The collected fractions from all 10 sample injections, containing detected compounds, were pooled, resulting in approximately 10 ml of sample per detected compound. Samples were concentrated 2× from approximately 10 ml to 5 ml by freeze-drying. Concentrated samples were tested for insecticidal activity in a DBM bioassay as described above. A negative control of mLB⁺ medium was used.

### Methanol Extraction of Bl45 Cultures and Electrospray Ionisation Mass Spectrometry

### Quenching

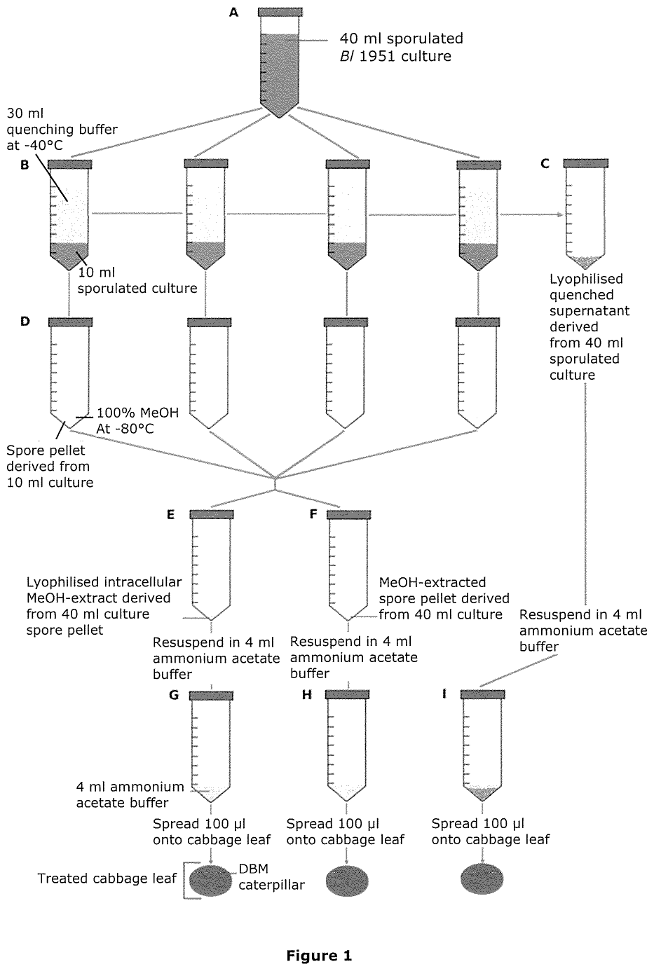

A culture of Bl45 was grown for 6 days in mLB⁺ medium until after sporulation as described above. Metabolic quenching of the culture, whereby the metabolism of the culture was halted, was followed by the cold MeOH-extraction of the culture pellet using methods adapted from Faijes et al. (2007). A volume of 40 ml sporulating culture was quenched at a 1:3 ratio of culture and buffer respectively, in quenching buffer (60% MeOH and 0.85% w/v ammonium carbonate; pH 8.5) kept at −40° C. The culture was incubated in quenching buffer for 30 minutes at −40° C. and then centrifuged for 5 minutes at −9° C. and 3000×g. The supernatant was kept apart on dry ice after which the pellet was washed in the same volume of quenching buffer at −40° C. to remove any accessory extracellular metabolites. The supernatants were pooled and diluted in an equal volume of ice cold MQW, frozen at −80° C., lyophilised and stored at −80° C. until further use (FIG. 1).

### Cold Methanol Extraction of the Bacterial Spore Pellet

The spore pellet was resuspended in 1 ml of −80° C. absolute MeOH and frozen in liquid nitrogen. The extract was subsequently thawed on ice and centrifuged at 10000×g, for two minutes at 4° C. The supernatant was kept apart on dry ice and the pellet was re-extracted two more times with 0.5 ml −80° C. absolute MeOH and twice in 0.5 ml ice cold MQW. All the extracts were pooled and diluted in an equal volume of ice cold water and immediately frozen in liquid nitrogen, lyophilised and stored at −80° C. until further use. The extracted spore pellet was also stored at −80° C. until further use (FIG. 1).

### Sample Preparation of the Methanol Extracts Prior to Bioassay Setup

The dry weights of the lyophilised intracellular and quenched supernatant samples were weighed using an analytical scale. The intracellular MeOH-extract, the quenched culture supernatant and MeOH-extracted spore pellet were resuspended in 4 ml of ammonium acetate buffer (50 mM ammonium acetate, pH 8.5), prior to being applied in a DBM bioassay. As all these extracts and the pellet were originally derived from a 40 ml sporulated culture, the extracts were thus 10× concentrated (FIG. 1). Ammonium acetate did not interfere with subsequent mass spectrometry analysis.

Three separate DBM larval bioassays were set up with as described above with the following treatments: 1) Bl45 original full strength sporulated culture, 2) 10× concentrated MeOH extracted spore pellet, 3) 10× concentrated intracellular extract, 4) 10× concentrated quenched supernatant. Ammonium acetate buffer (50 mM ammonium acetate buffer; pH 8.5), mLB⁺ medium (7.7 mM K₂HPO₄, 42 mM KH₂PO₄, 2.5% w/v LB, 0.0125% w/v NaOH, 5.25 mM NTA, 0.59 mM MgSO₄, 0.91 mM CaCl₂, 0.04 mM FeSO₄, 2.5 mM MnCl₂ and 1% w/v glucose; pH 7.6) and 10× concentrated mLB⁺ medium (77 mM K₂HPO₄, 420 mM KH₂PO₄, 25% w/v LB, 0.125% w/v NaOH, 52.5 mM NTA, 5.9 mM MgSO₄, 9.1 mM CaCl₂, 0.4 mM FeSO₄, 25 mM MnCl₂ and 10% w/v glucose) were used as negative controls.

### Protein Analysis of the Bioassay Treatments

Analysis of the protein profiles and protein concentrations of the bioassay treatments was conducted by SDS-PAGE and Bradford measurements respectively.

Mass Spectrometry of Putative Proteinaceous Toxins of Bl45 Derived from 10× Concentrated Extracellular Methanol Extracts

The sample of interest, the quenched culture supernatant, was run on 10 lanes of a 4-8% acrylamide/bis gel by SDS-PAGE. One half of the sample was run at a 1:5 dilution, and the other half was run at a 1:2.5 dilution on the remaining 5 lanes. The band of interest of about 60 kDa was excised from the gel in all 10 lanes using a sterile surgical knife. The bands were transferred to a 1.7 ml Eppendorf tube and suspended in 1.5 ml sterile MQW. Subsequently, the bands were analysed by ESI-mass spectrometry (Santanu Deb-Choudhury, AgResearch, Lincoln, New Zealand).

### In-Gel Trypsin Digest

A volume of 10 μl of the extract was separated on an 8% acrylamide/bis gel and stained with 0.05% CBB R250, 10% acetic acid, 15% methanol and 3% ammonium sulphate. A 60 kDa band was excised, cut into 1 mm pieces and destained at 37° C. for 1 hours with 200 μl of 200 mM NH₄HCO₃ in 50% acetonitrile. The destaining solution was then discarded. The gel slices were then reduced with 200 μl 50 mM tris (2-carboxyethyl) phosphine in 100 mM ammonium bicarbonate at 56° C. for 45 min. The reduction solution was discarded and the gel pieces were washed once with 100 mM NH₄HCO₃. Alkylation was performed with 100 μl of 150 mM iodoacetamide in 100 mM NH₄HCO₃ and vortexing for 30 min in the dark. The alkylation solution was then discarded and the gel pieces washed once with 50 mM NH₄HCO₃. Protein digestion was performed by adding 80 μl 50 mM NH₄HCO₃ containing 1.5 μg porcine trypsin (Promega, Madison, WI, USA) and 10% acetonitrile and incubating at 37° C. for 18 hours. The resulting peptides were extracted directly using a procedure as previously described (Koehn et al., 2011). Subsequent analysis of the peptides was performed using LC-MS/MS.

### LC-MS/MS for Protein Identification

LC-MS/MS was performed on a nanoAdvance UPLC coupled to an amaZon speed ETD ion trap mass spectrometer equipped with a CaptiveSpray ion source (Bruker Daltonik, Bremen, Germany) operated at 1400 V. Five μl of sample was loaded on a C18AQ Nanotrap (Bruker, C18AQ, 5 μm, 200 Å). The trap column was then switched in line with an in-house packed analytical column (100 μm ID×150 mm) containing Magic C18AQ (3 μm, 200 Å; Bruker). The column oven temperature was maintained at 50° C. A gradient elution was performed from 2% solvent A (0.1% formic acid) to 45% B (98% acetonitrile, 0.1% formic acid) in 60 min at a flow rate of 800 nl/min. The column outlet was directly interfaced to an amaZon speed ETD (Bruker) mass spectrometer equipped with a CaptiveSpray source. Automated information dependent acquisition (IDA) was performed using TrapControl (version 7.1. Build 83, Bruker) software, with a MS survey scan over the range m/z 350-1200 followed by three MS/MS spectra from m/z 50-2200 acquired during each cycle of 30 ms duration.

### Database Search

Peak list data were extracted using DataAnalysis v4.2 (Bruker) and imported into ProteinScape v3.1 (Bruker) for protein identification. Database searching was performed on an in-house Mascot v2.4 Server (Matrix Science, UK). The following MS/MS search parameters (amaZon speed ETD in CID mode) were used: database NCBI (29 May 2014); taxonomy Bacteria (Eubacteria); enzyme semitrypsin; two missed cleavage allowed; fixed modification carbamidomethyl (C); variable modifications oxidation (M); peptide tolerance 0.3 Da; MS/MS tolerance 0.6 Da; monoisotopic mass; instrument specificity: ESI-TRAP. ProteinScape—ProteinExtractor settings were: peptide acceptance threshold 40; protein acceptance threshold 80, with at least one significant peptide identity score calculated by the search engine required for protein identification.

### Bio-Informatic Analysis

Gene identification in the Bl45 genome using the peptides identified by ESI-mass spectrometry was conducted with Geneious 2 version 8.1.7 (Biomatters Ltd © 2005-2015). Protein homologs were identified with the Basic Local Alignment Search Tool x (BLASTx), from translated nucleotide to protein, of the National Centre for Biotechnology Information (NCBI), the U.S. National Library of Medicine (NLM) and the National Institutes of Health (NIH) in the non-redundant (nr) protein database using the Bacteria and Archaea (11) genetic code (Basic Alignment Search Tool, 2017). Gene homologs were identified using tBLASTn, from protein to translated nucleotide, of the NCBI, NLM and NIH in the nucleotide collection (nr/nt) database (Basic Alignment Search Tool, 2017). Enhanced lookup of potential conserved domains was conducted with Domain Enhanced Lookup Time Accelerated (DELTA) BLAST, from protein to protein, in the nr database (Basic Alignment Search Tool, 2017). Protein structure homology homologs were identified using SWISS-MODEL via the ExPASy web server from the Swiss Institute of bio-informatics (SIB) and the Biozentrum Centre for Molecular Life Sciences (Arnold et al., 2006; Biasini et al., 2014; Guex et al., 2009; Kiefer et al., 2009). The molecular weight of translated amino acid sequences was calculated with the protein molecular weight calculator of the Sequence Manipulation Suite (“Protein Molecular Weight Calculator,” 2017).

### Results

Gradient centrifugation of Bl45 sporulated cultures was performed to identify cellular and culture fractions associated with insect toxicity. There was an expectation that, like for certain reported Bacillus strains, insecticidal activity would be associated with proteinaceous crystalline inclusions, likely comprising Cry proteins.

Gradient fractions with detected protein levels, the culture supernatants of gradient separations, and the full strength Bl45 sporulated culture used in one of the gradient separations, were selected for a DBM larvae bioassay.

As shown in Table 1, none of the gradient bands showed any significant insecticidal activity toward the DBM larvae. The culture supernatants derived from gradient separations A and B had a significant cumulative mortality compared to the negative control and the gradient bands (P<0.001).

TABLE 1
Cumulative mortality (%) in the DBM bioassay of Bl45 gradient fractions.

| | | Cumulative | |
|---|---|---|---|
| | Protein | Mortality | |
| | concentration | (%) | Sig. of dif. |
| Treatment | (mg/ml) | Day 2 | with control^{a} |
| 1. Dipel Bt subsp. kurstaki 32000 U/ml | NM^{b} | (100.0) | *** |
| 2. Negative control (sterile MQW) | — | 6.7 | — |
| 3. Bl 1951 full strength culture B | NM | 80.0 | *** |
| 4. Bl 1951 supernatant separation A | 0.90 | 86.7 | *** |
| 5. Bl 1951 supernatant separation B | 0.91 | 46.7 | * |
| 6. Separation C, gradient band 1 | 0.05 | 6.7 | NS |
| 7. Separation C, gradient band 2 | 0.32 | 6.7 | NS |
| 8. Separation C, gradient band 3 | 0.38 | 20.0 | NS |
| 9. Separation C, gradient band 4 | ND^{c} | 20.0 | NS |
| 10. Separation A, gradient band 1 | 0.67 | 13.3 | NS |
| 11. Separation B, gradient band 5 | 2.3 | (0.0) | NS |
| LSD 5% | | 35.25 | |
| LS effect 5% | | 24.93 | |
| (LSD 5%/✓2) | | | |
| Overall P-value | | <0.001 | |

^{a}Significance of difference with control. NS = Not Significant; * = P < 0.05; *** = P < 0.001.
^{b}NM = Not measured.
^{c}ND = Not detectable.

Table 1 shows the cumulative mortality (%) at day 2 of the DBM bioassay with Bl45 gradient fractions. Treatments that are constant, 0 or 100, have been omitted from the ANOVA (means are in brackets). To compare two un-bracketed means, the LSD was used. To compare a bracketed mean with an un-bracketed mean, the LS Effect was used.

These results indicated that, unexpectedly, the main insecticidal activity of Bl45 toward DBM larvae was not located in the spores or the supposed crystals, but in the culture supernatant.

### Example 2: Identification of Insecticidal Activity

This example presents experiments to identify an insecticidal activity from Brevibacillus laterosporus strain NMI No. V12/001945 (Bl45).

The results presented in Example 1 suggested that the main insecticidal activity of Bl45 was located within the culture supernatant. To identify insecticidal protein toxins within the culture, the proteins in the culture supernatant were precipitated with ammonium sulphate, concentrated and separated by size exclusion chromatography.

Proteins generally denature at temperatures above 80° C. and aggregate, resulting in the loss of bioactivity. A sample of the culture supernatant was heated to get an indication of the molecular nature of the larvicidal toxins present in the culture supernatant, for example whether the insecticidal activity comprised protein or a secondary metabolite.

The size exclusion fractions, original full strength culture, unwashed spores and unheated and heated culture supernatant were tested for activity against DBM larvae. Additionally, filtered culture supernatant derived from another sporulated culture of Bl45 was included in the DBM bioassay. This additional sample (referred to below as FCS-D) was sent for further bioassay, and was also tested for DBM larvae activity.

### Results

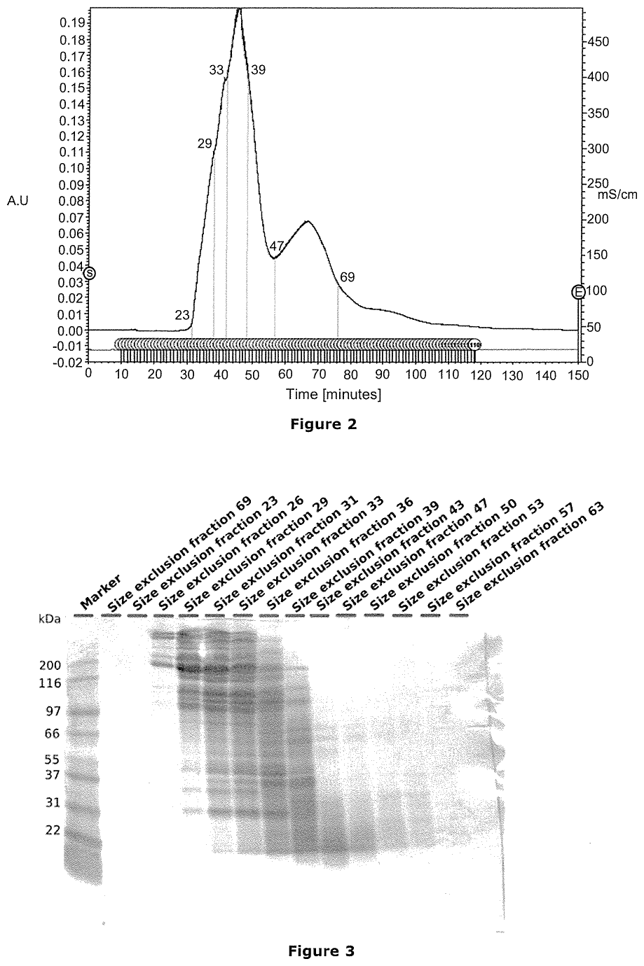

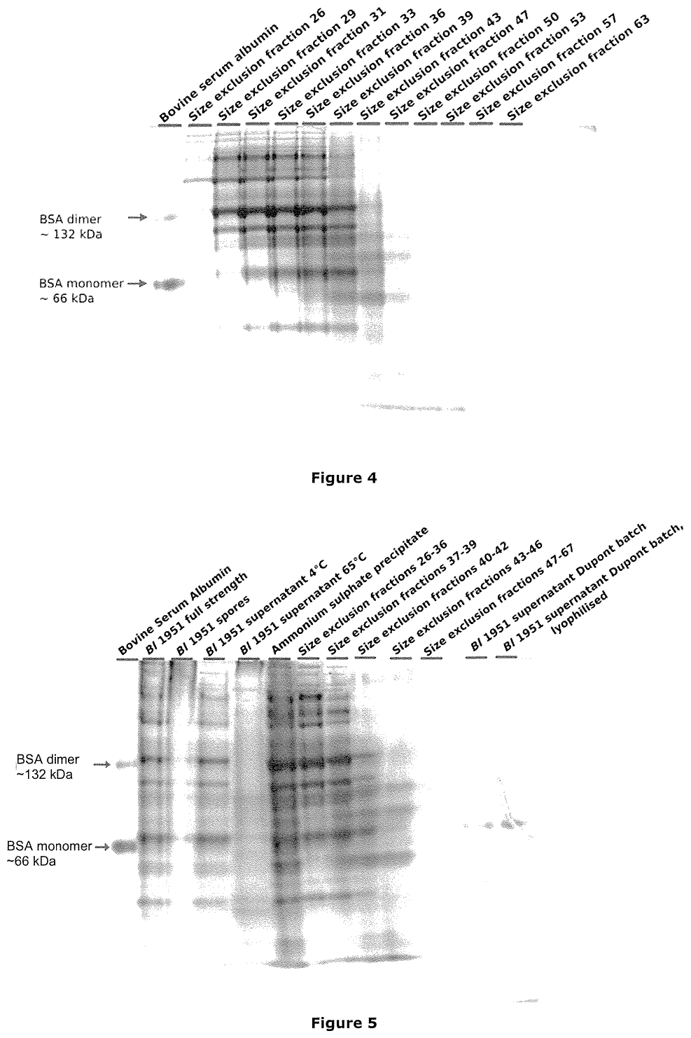

The size exclusion chromatography yielded two distinct peaks (FIG. 2). Protein analysis of the peak fractions showed a poor separation with a generally high number of bands in each fraction (FIGS. 3 and 4). Fractions with similar protein profiles were pooled to test for activity in a DBM larvae bioassay. The DBM bioassay results reveal that the size exclusion fractions did not have any significant lethal activity (Table 2).

TABLE 2
Cumulative mortality (%) at day 4 of the DBM bioassay
with Bl45 size exclusion chromatography fractions.

| | Protein | Cumulative | Sig. of |
|---|---|---|---|
| | concentration | Mortality (%) | dif. with |
| Treatment | (mg/ml) | Day 4 | control^{a} |
| 1. Dipel Bt subsp. kurstaki | NM^{b} | (100.0) | *** |
| 2. Negative control (sterile MQW) | − | 20.0 | − |
| 3. Bl45 full strength culture | 5.5 | 60.0 | + |
| 4. Bl45 unwashed spore suspension | 3.6 | 80.0 | ** |
| 5. Bl45 supernatant 4° C. | 7.0 | 60.0 | + |
| 6. Bl45 supernatant 65° C. | 6.3 | 20.0 | NS |
| 7. Bl45 supernatant ammonium sulphate | 8.5 | 66.7 | * |
| precipitate | | | |
| 8. Size exclusion fractions 26-36 | 6.9 | 6.7 | NS |
| 9. Size exclusion fractions 37-39 | 4.7 | (0.0) | NS |
| 10. Size exclusion fractions 40-42 | 3.9 | (0.0) | NS |
| 11. Size exclusion fractions 43-47 | 3.9 | 26.7 | NS |
| 12. Size exlusion fractions 48-67 | 0.1 | 26.7 | NS |
| 13. FCS-D | ND^{c} | (100.0) | *** |
| 14. FCS-D, lyophilised | ND | (100.0) | *** |
| LSD 5% | | 43.11 | |
| LS effect 5% (LSD 5%/✓2) | | 30.48 | |
| LSD 10% | | 35.51 | |
| LS effect 10% | | 25.11 | |
| (LSD 10%/✓2) | | | |
| Overall P-value | | 0.021 | |

^{a}Significance of difference with control. NS = Not Significant; + = P < 0.1; * = P < 0.05; ** = P < 0.01; *** = P < 0.001.
^{b}NM = not measured.
^{c}ND = non detectable.

Table 2 shows the cumulative mortality (%) at day 4 of the DBM bioassay with Bl45 size exclusion chromatography fractions. Treatments that are constant, 0 or 100, have been omitted from the ANOVA (means are in brackets). To compare two un-bracketed means, the LSD was used. To compare a bracketed mean with an un-bracketed mean, the LS Effect was used.

The unwashed spores and ammonium sulphate precipitate had a significantly higher cumulative mortality than the other Bl45 treatments when the LSD was 5% (P=0.021). The full strength culture and the unheated culture supernatant, unwashed spores and ammonium sulphate precipitate had a significantly higher cumulative mortality when the LSD was 10% (P=0.021). The heated culture supernatant lost a lot of activity and had no significant mortality, indicating that the insecticidal compounds in the culture supernatant were heat sensitive and most likely of proteinaceous nature.

The FCS-D samples were not included in the ANOVA because of their constant values on day four, where the mortality was 100%, but the results could be interpreted using the LS Effect. The FCS-D samples had a significantly higher mortality than the other treatments, except for the unwashed spores and Dipel, with both 5% and 10% LS Effect levels (Table 2, P=0.021). This was unexpected, given the low number of protein bands observable in these samples by SDS-PAGE (FIG. 5) and the fact that no proteins could be detected by Bradford measurement.

The results of this experiment showed a loss of activity when the culture supernatant was heated at 65° C., suggesting that the insecticidal compounds within the supernatant were likely of proteinaceous nature. The size exclusion fractions did not display any significant activity, however.

Additionally, the FCS-D samples were highly toxic and had an undetectable amount of protein. This suggested that at least some of the high insecticidal activity observed with the FCS-D samples was likely not caused by proteins, but rather another type of biomolecule.

Notably, in this experiment the spores had a high lethal activity, unlike in the previous gradient centrifugation experiment, suggesting that a considerable level of insecticidal toxicity was located in the spores.

### Example 3: Identification of Insecticidal Activity

This example presents experiments to identify an insecticidal activity from Brevibacillus laterosporus strain NMI No. V12/001945 (Bl45).

The results presented in the preceding examples suggested that substantial insecticidal activity of Bl45 was located within the culture supernatant and in the unwashed spores.

It was unclear however whether this activity was caused by toxins of proteinaceous nature or by other biomolecules, or a combination, because the proteins in the size exclusion fractions did not show any activity toward DBM larvae. In contrast, a second batch of Bl45 filtered culture supernatant, FCS-D, was highly active to the DBM larvae. The FCS-D samples had neglible protein content. Additionally, significant high activity was also observed within the spore suspension of Bl45 (see Table 2).

As these spores were unwashed, it was unclear whether washed spores were also lethal to DBM larvae.

Four bioassays were conducted to further explore the nature of the insecticidally active compounds in the culture supernatant, and to further establish whether the Bl45 spores were toxic to DBM larvae.

Bioassays were set up with sporulated full strength cultures, spores washed three times in sterile water, unheated culture supernatant and culture supernatant heated at 65° C. and 95° C.

### Results

The results of the first bioassay showed a highly lethal activity within the Bl45 FCS-D culture supernatant batches, comparable to Bt subsp. kurstaki (Table 3). Heating the FCS-D samples at 65° C. or 95° C. did not have any impact on the larvicidal DBM activity observed, compared to unheated FCS-D samples. This suggested that the toxin activity was heat stable at 95° C., and thus unlikely to be proteinaceous.

TABLE 3
Cumulative mortality (%) in the DBM bioassay with Bl45 culture fractions.

| | | | | Sig. of dif. with | |
|---|---|---|---|---|---|
| | Cumulative mortality (%) | | | control^{a} | |
| Treatment | Day 3 | Day 4 | Day 5 | Day 3 | Day 4 |
| 1. Dipel Bt subsp. kurstaki | (100.0) | (100.0) | (100.0) | *** | *** |
| 2. Negative control (sterile MQW) | 13.3 | 20.0 | 26.7 | − | − |
| 3. Bl45 full strength culture | 80.0 | 93.3 | 93.3 | ** | ** |
| 4. Bl45 spore suspension | 13.3 | 40.0 | 46.7 | NS | NS |
| 5. Bl45 supernatant 4° C. | 47.6 | 59.0 | 63.8 | + | + |
| 6. Bl45 supernatant 65° C. | 20.0 | 40.0 | 60.0 | NS | NS |
| 7. Bl45 supernatant 95° C. | 13.3 | 13.3 | 33.3 | NS | NS |
| 8. Bl45 supernatant 4° C., FCS-D | (100.0) | (100.0) | (100.0) | *** | *** |
| 9. Bl45 supernatant 65° C., FCS-D | (100.0) | (100.0) | (100.0) | *** | *** |
| 10. Bl45 supernatant 95° C., FCS-D | (100.0) | (100.0) | (100.0) | *** | *** |
| LSD 5% | 38.59 | 44.32 | 51.50 | | |
| LS Effect 5% (LSD5 %/✓2) | 27.29 | 31.34 | 36.42 | | |
| LSD 10% | 31.97 | 36.05 | 41.90 | | |
| LS Effect 10% (LSD 10%/✓2) | 22.61 | 25.49 | 29.60 | | |
| Overall P-value | 0.015 | 0.025 | 0.138 | | |

^{a}Significance of difference with control. NS = Not Significant; + = P < 0.1; ** = P < 0.01; *** = P < 0.001.

Table 3 shows the cumulative mortality (%) on days 3, 4 and 5 of the DBM bioassay with Bl45 culture fractions. Treatments that are constant, 0 or 100, have been omitted from the ANOVA (means are in brackets). To compare two un-bracketed means, the LSD is used. To compare a bracketed mean with an un-bracketed mean, the LS Effect is used.

In contrast, the cumulative mortality of the culture supernatants derived from another culture of Bl45 when heated at 65° C. and 95° C. did not differ significantly from the negative control, and neither did that observed with the washed spore suspension.

The cumulative mortality of the unheated supernatant, was significantly higher than the negative control when the LSD was 10% (Table 3). The cumulative mortality of the unheated culture supernatant did not differ significantly to that of the culture supernatant heated at 65° C. However, it did differ significantly with the culture supernatant heated at 95° C. The full strength culture had a significantly higher mortality than the negative control and the other treatments, except for the unheated culture supernatant (Table 3).

These results suggest that the principal insecticidal activity may vary between cultures of Bl45, with sometimes strong heat stability of toxic compounds within the culture supernatant, and with heat sensitive toxic compounds.

The results of the second bioassay showed a significant decrease of the cumulative mortality in the heated culture supernatants, derived from two different sporulated cultures of Bl45 (Table 4).

TABLE 4
Cumulative mortality (%) in the DBM larvae bioassay with Bl45 culture fractions.

| | Cumulative mortality (%) | | Sig. of dif. with control^{a} | |
|---|---|---|---|---|
| Treatments | Day 4 | Day 5 | Day 4 | Day 5 |
| 1. Dipel Bt subsp. kurstaki | (100.0) | (100.0) | *** | *** |
| 2. Negative control (sterile MQW) | 13.3 | 13.3 | — | — |
| 3. Bl45 full strength culture, batch 1 | 80.0 | 93.3 | ** | *** |
| 4. Bl45 3X washed spores batch 1 | 13.3 | 13.3 | NS | NS |
| 5. Bl45 supernatant 4° C., batch 1 | 66.7 | 80.0 | * | ** |
| 6. Bl45 supernatant 65° C., batch 1 | 20.0 | 20.0 | NS | NS |
| 7. Bl45 supernatant 95° C., batch 1 | 6.7 | 6.7 | NS | NS |
| 8. Bl45 full strength culture, batch 2 | 73.3 | 73.3 | ** | ** |
| 9. Bl45 3X washed spores, batch 2 | 20.0 | 20.0 | NS | NS |
| 10. Bl45 supernatant 4° C., batch 2 | 60.0 | 73.3 | * | ** |
| 11. Bl45 supernatant 65° C., batch 2 | 6.7 | 6.7 | NS | NS |
| 12. Bl45 supernatant 95° C., batch 2 | (0.0) | (0.0) | NS | NS |
| LSD 5% | 40.59 | 37.99 | | |
| LS Effect 5% (LSD 5%/✓2) | 28.70 | 26.86 | | |
| Overall P-value | 0.002 | <0.001 | | |

^{a}Significance of difference with control. NS = Not Significant; * = P < 0.05; ** = P < 0.01; *** = P < 0.001.

Table 4 shows the cumulative mortality (%) at day 4 of the DBM larvae bioassay with Bl45 culture fractions. Treatments that are constant, 0 or 100, have been omitted from the ANOVA (means are in brackets). To compare two un-bracketed means, the LSD is used. To compare a bracketed mean with an un-bracketed mean, the LS Effect is used.

The washed spores of both cultures were not significantly active toward the DBM larvae compared to the negative control. The unheated culture supernatants of both cultures and the full strength cultures had significantly higher mortality rates than the negative control, and did not differ significantly from each other. These results indicated considerable heat-sensitivity of a principal active toxin(s) present within the culture supernatant of both cultures.

The third and fourth bioassays were repeats of each other and again showed a considerable degree of heat-stability in the culture supernatants heated at 95° C. (see Tables 5 and 6).

TABLE 5
Cumulative mortality (%) in the DBM larvae
bioassay with Bl45 culture fractions.

| | Cumulative | |
|---|---|---|
| | Mortality (%) | Sig. of dif. |
| Treatment | Day 4 | with control^{a} |
| 1. Dipel Bt subsp. kurstaki | (100.0) | *** |
| 2. Negative control (sterile MQW) | 5.0 | — |
| 3. Bl45 full strength culture | 85.0 | *** |
| 4. Bl45 3X washed spores | 20.0 | NS |
| 5. Bl45 supernatant 4° C. | 85.0 | *** |
| 6. Bl45 supernatant 95° C. | 35.0 | * |
| LSD 5% | 24.52 | |
| LS Effect 5% (LSD 5%/✓2) | 17.34 | |
| Overall P-value | < 0.001 | |

^{a}Significance of difference with control. NS = Not Significant; * = P < 0.05; *** = P < 0.001.

Table 5 shows the cumulative mortality (%) at day 4 of the DBM larvae bioassay with Bl45 culture fractions. Treatments that are constant, 0 or 100, have been omitted from the ANOVA (means are in brackets). To compare two un-bracketed means, the LSD is used. To compare a bracketed mean with an un-bracketed mean, the LS Effect is used.

The results of the third bioassay showed a low but significant degree of heat-sensitivity in the heated culture supernatant (Table 5). The insecticidal activity decreased in the heated culture supernatant and differed significantly in cumulative mortality compared to the unheated culture supernatant (P<0.001). The cumulative mortality of the heated culture supernatant was significantly higher compared to the negative control, however (P<0.001). The washed spores did not differ significantly in mortality compared to the negative control. The mortality rates of the unheated culture supernatant and the full strength culture were significantly higher than that of the negative control, and did not differ significantly from each other (P<0.001).

TABLE 6
Cumulative mortality (%) in the DBM larvae bioassay with Bl45 culture fractions.

| | Cumulative mortality | | Sig. of dif. with control^{a} | |
|---|---|---|---|---|
| Treatment | Day 3 | Day 4 | Day 3 | Day 4 |
| 1. Dipel Bt subsp. kurstaki | (100.0) | (100.0) | *** | *** |
| 2. Negative control (sterile MQW) | (0.0) | (0.0) | — | — |
| 3. Bl45 full strength culture | 70.0 | 70.0 | *** | *** |
| 4. Bl45 3X washed spores | 10.0 | 10.0 | NS | NS |
| 5. Bl45 supernatant 4° C. | 45.0 | 65.0 | ** | *** |
| 6. Bl45 supernatant 95° C. | 45.0 | 50.0 | ** | ** |
| LSD 5% | 34.14 | 37.80 | | |
| LS Effect 5% (LSD 5%/✓2) | 24.14 | 26.73 | | |
| Overall P-value | 0.022 | 0.022 | | |

^{a}Significance of difference with control. NS = Not Significant; ** = P < 0.01; *** = P < 0.001.

Table 6 shows the cumulative mortality (%) at day 4 of the DBM larvae bioassay with Bl45 culture fractions. Treatments that are constant, 0 or 100, have been omitted from the ANOVA (means are in brackets). To compare two un-bracketed means, the LSD is used. To compare a bracketed mean with an un-bracketed mean, the LS Effect is used.

The results of the fourth bioassay were comparable to that of the third bioassay, except for the considerably higher level of heat-resistance within the heated culture supernatant (Table 6) that was observed. The heated culture supernatant did not differ significantly to that of the unheated supernatant (Table 6).

The results of these two bioassays again demonstrated that at least part of the insecticidal activity present within the culture supernatant showed a considerable level of heat-stability.

In summary, this series of bioassays demonstrated that the main insecticidal activity of Bl45 was located within the culture supernatant. No significant activity was observed in the washed spore fractions.

A significant level of activity toward DBM larvae was still observed in the heated culture supernatant fractions, tested in three separate bioassays (Tables 3, 5 and 6). However, a significant decrease of DBM activity within heated culture supernatants was observed in three separate bioassays as well (Tables 3, 4 and 5), with no significantly higher mortality rates compared to the negative control in two of these bioassays (Tables 3 and 4).

These results suggest that the toxicity of Bl45 toward DMB larvae may vary with different cultures or culturing conditions. Additionally, the results indicate that the insecticidal compounds located within the culture supernatant might consist of a mixture of heat-sensitive and heat-stable biomolecules, produced and excreted by Bl45. There is a possibility that these biomolecules differ in their molecular nature, with the heat-sensitive molecules likely being proteinaceous.

### Example 4: Cellular Location of Insecticidal Activity

This example presents further experiments to identify an insecticidal activity from Brevibacillus laterosporus strain NMI No. V12/001945 (Bl45).

The results presented in the preceding examples suggested that substantial insecticidal activity of Bl45 was located within the culture supernatant, and that there was the possibility that activities of a proteinaceous nature, and of a non-proteinaceous nature, may be present.

The relative heat-stability of the toxicity toward DBM larvae observed in the heated culture supernatant of some cultures of Bl45 was reminiscent of the non-proteinaceous, thermo-stable and broad-spectrum active beta-exotoxin, also know as thuringiensin, produced by some strains of Bt (Palma et al., 2014).

An experiment was conducted to characterise the possible secondary metabolite toxin from the culture supernatant of Bl45 by reversed-phase high pressure liquid chromatography (RP-HPLC).

### Results

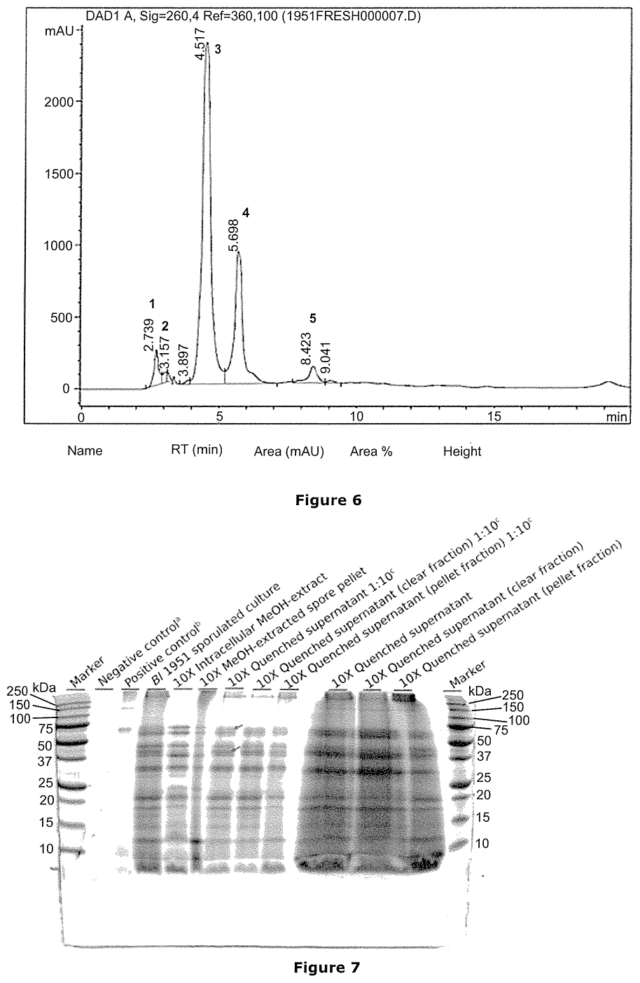

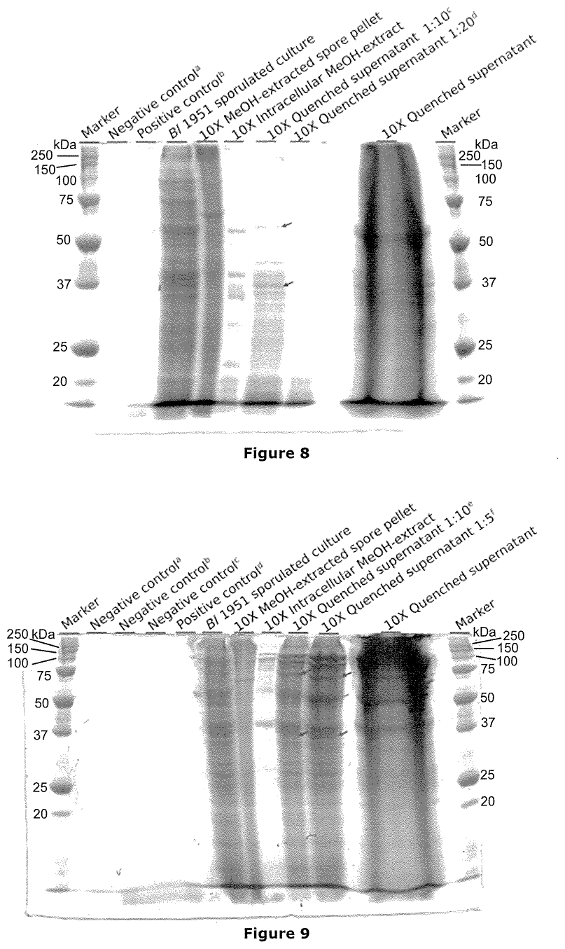

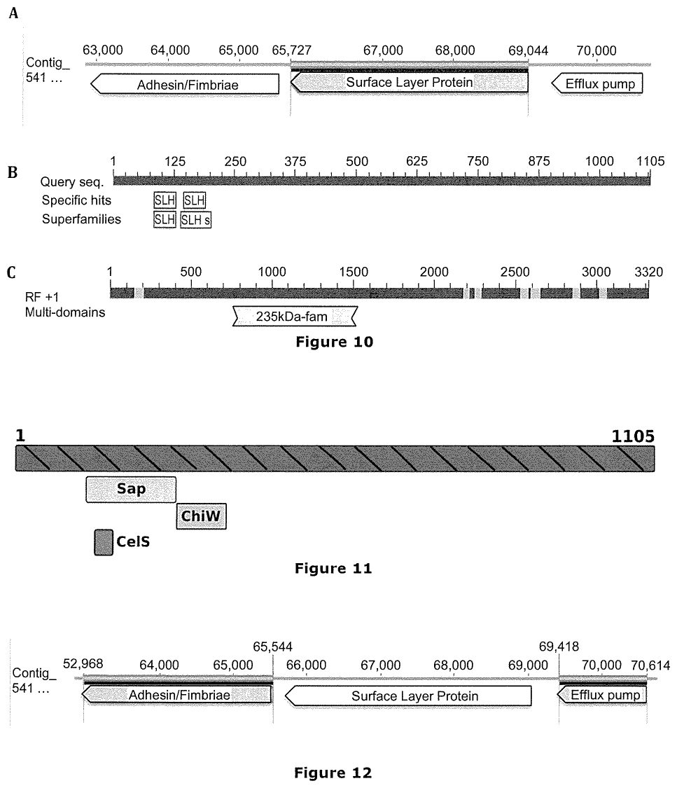

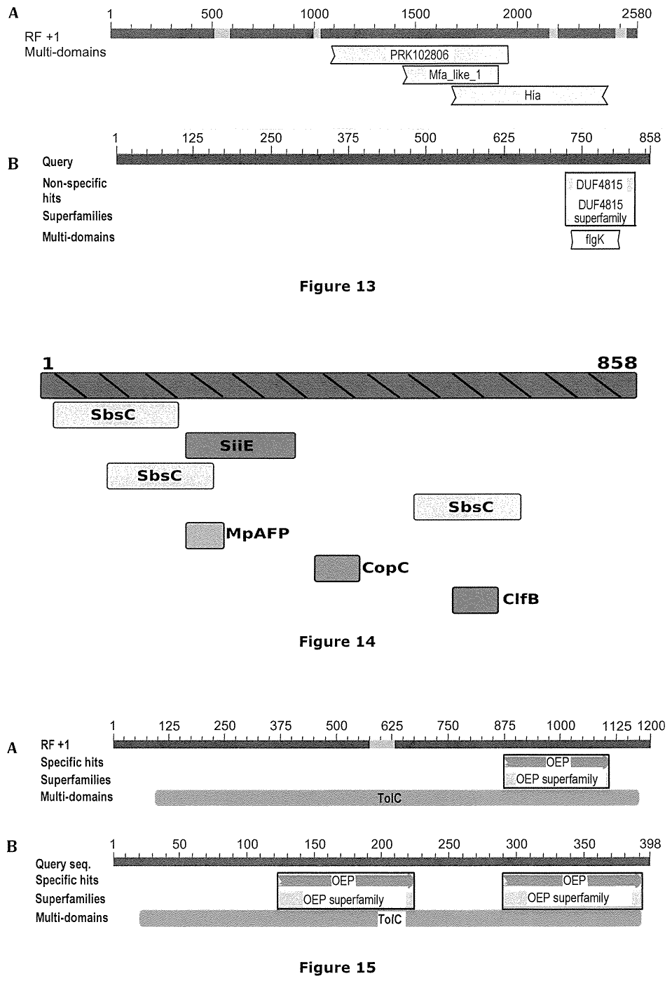

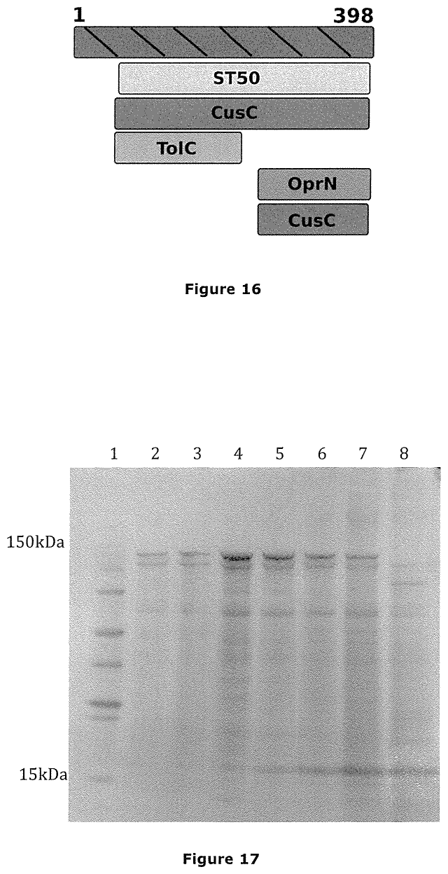

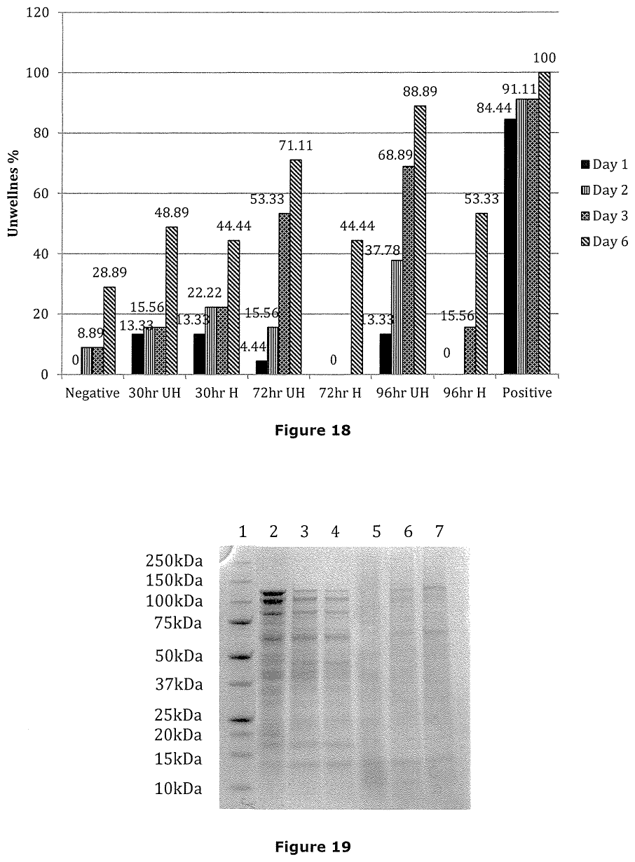

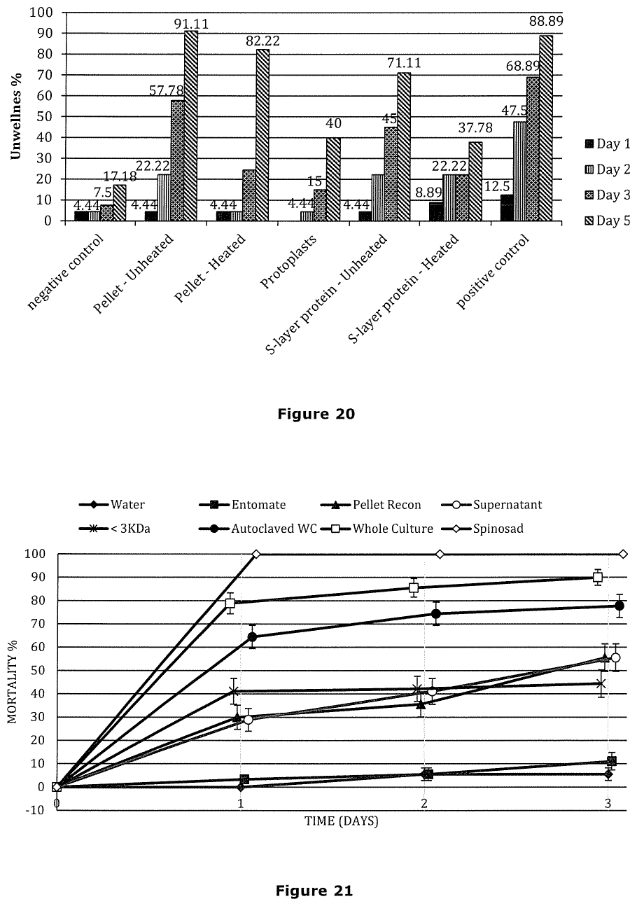

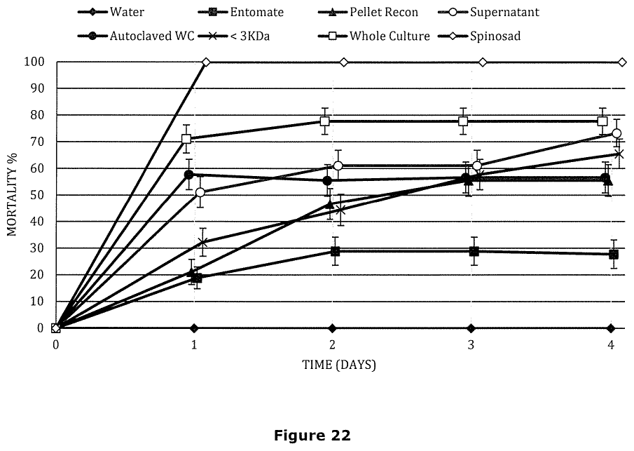

The RP-HPLC of Bl45 culture supernatant yielded five separate peaks (FIG. 6). The pooled RP-HPLC fractions were tested for insecticidal activity against DBM larvae in a single bioassay, along with the original full strength culture and crude culture fractions. The bioassay results revealed no significant cumulative mortality of the RP-HPLC fractions, culture supernatant heated at 95° C., or the washed spores, compared to the negative control (Table 7).

TABLE 7
Cumulative mortality (%) in the DBM larvae bioassay
with Bl45 crude culture fractions and RP-HPLC fractions.

| | Protein | Cumulative | |
|---|---|---|---|
| | concen- | mortality | Sig. of |
| | tration | (%) | dif. with |
| Treatment | (mg/ml) | Day 3 | control^{a} |
| 1. Dipel Bt subsp. kurstaki | NDb | (100.0) | ** |
| 2. Negative control (mLB⁺ | − | 26.7 | − |
| medium) | | | |
| 3. Bl45 full strength culture | 1.8 | 80.0 | * |
| 4. Bl45 3X washed spores | ND | 20.0 | NS |
| 5. Bl45 supernatant 4° C. | 0.4 | 60.0 | + |
| 6. Bl45 supernatant 95° C. | ND | 13.3 | NS |
| 7. RP-HPLC fraction 1 | ND | 13.3 | NS |
| 8. RP-HPLC fraction 2 | ND | 26.7 | NS |
| 9. RP-HPLC fraction 3 | ND | 26.7 | NS |
| 10. RP-HPLC fraction 4 | ND | 26.7 | NS |
| 11. RP-HPLC fraction 5 | ND | 6.7 | NS |
| LSD 5% | | 40.00 | |
| LS Effect 5% (LSD 5%/✓2) | | 28.28 | |
| LSD 10% | | 33.01 | |
| LS Effect 10% (LSD 10%/✓2) | | 23.34 | |
| Overall P-value | | 0.028 | |

^{a}Significance of difference with control. NS = Not Significant; + = P < 0.1; * = P < 0.05; *** = P < 0.001.
^{b}ND = non detectable.

Table 7 shows the cumulative mortality (%) on day 3 of the DBM larvae bioassay with Bl45 crude culture fractions and RP-HPLC fractions. Treatments that are constant, 0 or 100, have been omitted from the ANOVA (means are in brackets). To compare two un-bracketed means, the LSD is used. To compare a bracketed mean with an un-bracketed mean, the LS Effect is used.

The original full strength culture had a significantly higher mortality compared to the negative control, the RP-HPLC fractions, the heated culture supernatant, and the washed spores (Table 7). The cumulative mortality of the full strength culture did not differ significantly to that of the unheated culture supernatant, however (Table 7). The unheated culture supernatant had a significantly higher mortality compared to the heated culture supernatant, the RP-HPLC fractions 1 and 5, and the washed spores (Table 7). The cumulative mortality of the unheated culture supernatant was significantly higher compared to the negative control, RP-HPLC fractions 2-4 at the LSD 10% level (Table 7).

The observed heat-sensitivity of the original culture supernatant suggests that the presumed heat-stable secondary metabolites were not present in this sample.

Additionally, no proteins were detected in the inactive RP-HPLC fractions, the heated culture supernatant and the washed spores (Table 7). Proteins were, on the other hand, detected in the active full strength culture; 1.8 mg/ml, and in the unheated culture supernatant; 0.4 mg/ml.

In summary, the results suggest that the observed toxicity of the full strength culture and unheated culture supernatant was likely caused by toxins of proteinaceous nature, and not by beta-exotoxin like secondary metabolites.

### Example 5: Identification of Insecticidal Activity

This example presents further experiments to identify an insecticidal activity from Brevibacillus laterosporus strain NMI No. V12/001945 (Bl45).

The results presented in the preceding examples suggested that substantial insecticidal activity of Bl45 was located within the culture supernatant, and that there was the possibility that activities of a proteinaceous nature, and of a non-proteinaceous nature, may be present.

A series of methanol (MeOH) extractions of sporulated Bl45 cultures were conducted to isolate and identify the DBM-active toxins.

Both the culture supernatant and the spore pellets underwent a MeOH-extraction and were separately tested against DBM larvae. A screening bioassay with four cultures, all cultured under different conditions, was conducted to find the best conditions for high volume culturing, and to select the most active culture to be used in the first MeOH-extraction (Table 8).

TABLE 8
Cumulative mortality (%) in the DBM larvae bioassay with
Bl45 full strength cultures and 10 X diluted cultures.

| | Cumulative | | Sig. of dif. | |
|---|---|---|---|---|
| | mortality (%) | | with control^{a} | |
| Treatment | Day 2 | Day 3 | Day 2 | Day 3 |
| 1. Dipel Bt subsp. kurstaki | (100.0) | (100.0) | *** | *** |
| 2. Negative control (mLB⁺ medium) | 6.7 | 26.7 | − | − |
| 3. Culture 1, Bl45 full strength (100 ml/L) | 86.7 | 93.3 | *** | ** |
| 4. Culture 1, Bl45 1:10 dilution (100 ml/L) | (0.0) | (0.0) | NS | NS |
| 5. Culture 2, Bl45 full strength (200 ml/L) | 46.7 | 66.7 | * | + |
| 6. Culture 2, Bl45 1:10 dilution (200 ml/L) | 6.7 | 6.7 | NS | NS |
| 7. Culture 3, Bl45 full strength (200 ml/2 L) | 46.7 | 46.7 | * | NS |
| 8. Culture 3, Bl45 1:10 dilution (200ml/2 L) | 13.3 | 13.3 | NS | NS |
| 9. Culture 4, Bl45 full strength (400 ml/2 L) | 53.3 | 66.7 | * | + |
| 10. Culture 4, Bl45 1:10 dilution (400 ml/2 L) | 26.7 | 33.3 | NS | NS |
| LSD 5% | 36.55 | 41.86 | | |
| LS Effect 5% (LSD 5%/✓2) | 25.84 | 29.60 | | |
| LSD 10% | 30.02 | 34.38 | | |
| LS Effect 10% (LSD 10%/✓2) | 21.23 | 24.31 | | |
| Overall P-value | 0.004 | 0.007 | | |

^{a}Significance of difference with control. NS = Not Significant; + = P < 0.1; * = P < 0.05; ** = P < 0.01; *** = P < 0.001.

Table 8 shows the cumulative mortality (%) on day 2 of the DBM larvae bioassay with Bl45 full strength cultures and 10× diluted cultures. Treatments that are constant, 0 or 100, have been omitted from the ANOVA (means are in brackets). To compare two un-bracketed means, the LSD is used. To compare a bracketed mean with an un-bracketed mean, the LS Effect is used.

The results of this bioassay reveal that culture 1, 100 ml of medium in a 1 L flask (100 ml/L), had the highest activity, and had a significantly higher mortality than cultures 2 (200 ml/L) and 3 (200 ml/2 L) (Table 8). Culture 1 had a significantly higher cumulative mortality compared to culture 4 (400 ml/2 L) at the LSD 10% level. Culture 1 had the highest spore count as well, and was selected for a subsequent MeOH-extraction. The MeOH-extracted samples were concentrated twice before testing against DBM larvae. The bioassay results showed no significant activity in any of the Bl45 treatments compared to the negative control (Table 9).

TABLE 9
Cumulative mortality (%) in the DBM larvae bioassay with Bl45 methanol extracts.

| | | | | | Sig. of |
|---|---|---|---|---|---|
| | Protein | | | | dif. with |
| | conc. | Cumulative mortality (%) | | | control^{b} |
| Treatment | (mg/ml) | Day 3 | Day 4 | Day 5 | Day 5 |
| 1. Dipel Bt subsp. kurstaki | ND^{a} | (100.0) | (100.0) | (100.0) | *** |
| 2. Negative control (50 mM | — | 6.7 | 12.2 | 12.2 | — |
| ammonium acetate; pH 8.5) | | | | | |
| 3. Bl45 full strength culture | 1.6 | 40.0 | 46.7 | 53.3 | NS |
| 4. Bl45 intracellular extract | ND | 6.7 | 20.0 | 20.0 | NS |
| 5. Bl45 MeOH-extracted spore pellet | ND | 6.7 | 6.7 | 26.7 | NS |
| 6. Bl45 quenched supernatant | 1.0 | 6.7 | 6.7 | 20.0 | NS |
| 7. Bl45 quenched supernatant (pellet) | 0.4 | 33.3 | 33.3 | 33.3 | NS |
| LSD 5% | | 41.49 | 34.73 | 45.71 | |
| LS Effect 5% (LSD 5%/√2) | | 29.34 | 24.56 | 32.32 | |
| Overall P-value | | 0.300 | 0.143 | 0.469 | |

^{a}ND = non detectable.
^{b}Significance of difference with control.
NS = Not Significant;
*** = P < 0.001.

Table 9 shows the cumulative mortality (%) on days 3, 4 and 5 of the DBM larvae bioassay with Bl45 methanol extracts. Treatments that are constant, 0 or 100, have been omitted from the ANOVA (means are in brackets). To compare two un-bracketed means, the LSD is used. To compare a bracketed mean with an un-bracketed mean, the LS Effect is used.

To evoke a significant level of toxicity toward DBM larvae, a series of three MeOH-extractions were performed wherein the MeOH-extracts were concentrated 10 times before testing against DBM larvae. Three separate bioassays were conducted. The results of the first bioassay showed that the larval mortality of the 10× concentrated quenched supernatant was significantly higher than the other Bl45 treatments, and did not differ significantly to the positive control on day one (Table 10). Prior to the bioassay, the quenched supernatant was divided in two fractions by centrifugation, the clear fraction and the pellet. Both of these fractions had significantly low larval mortalities compared to the un-separated supernatant on day one, but increased in activity over time. By day five, there was no significant difference in larval mortality between the original un-separated quenched supernatant and between the clear and pellet quenched supernatant samples (P=<0.001). The protein levels of the three active quenched supernatant samples were considerably higher than that of the other MeOH-extracts and the original full strength culture (Table 14). SDS-PAGE of the bioassay treatments from all three bioassay repeats showed two unique bands of approximately 40 kDa and 60 kDa in the quenched supernatant samples (FIGS. 7, 8 and 9). The quenched supernatant of the third bioassay, repeat three of three, did have an additional unique band of approximately 50 kDa, however (FIG. 9).

The second and third bioassay repeats showed comparable results to that of the first bioassay (Tables 11 and 12). Both 10× concentrated quenched supernatant samples had significantly higher larval mortalities from day one after incubation compared to the other Bl45 treatments (Table 11 and Table 12), and reached 100% larval mortality on day two. The quenched supernatant samples were not tested in a separate clear fraction and pellet fraction against the DBM larvae in bioassay repeats two and three, because there was no difference found in the protein profiles of the clear and pellet supernatant fractions used in bioassay repeat one.

The larval mortality from the MeOH-extracted spores and the intracellular MeOH-extract was not significantly higher than the negative controls and the other Bl45 treatments, in both bioassay repeats two and three (Table 13).

In both bioassay repeats two and three, a brown discolouration of the cabbage leaves was observed in the insect active treatments; the full strength culture and the 10× concentrated quenched supernatant (data not shown). The larval cadavers had turned brown and soggy as well. Additionally, the leaves coated with the 10× concentrated quenched supernatant samples became covered in fungal colonies after three days of incubation (photos not shown).

The average larval mortality for all three bioassays combined was significantly highest for the quenched supernatant samples at 91.1% (Table 13). The average larval mortality of the intracellular MeOH-extracts and of the MeOH-extracted spores did not differ significantly to that of the negative control (P<0.001). The average larval mortality of the full strength cultures was significantly higher than that of the negative control, the intracellular MeOH-extracts and the MeOH-extracted spore pellets, but was significantly lower than that of the quenched supernatant samples at 49.3% (P<0.001).

TABLE 10
Cumulative mortality (%) in the DBM bioassay with BI45 10X concentrated methanol extracts.

| | Protein | | | | | | Sig. of dif. | |
|---|---|---|---|---|---|---|---|---|
| | conc. | Cumulative mortality (%) | | | | | with control^{a} | |
| Treatment | (mg/ml) | Day 1 | Day 2 | Day 3 | Day 4 | Day 5 | Day 2 | Day 5 |
| 1. Dipel Bt subsp. kurstaki | 0.11 | 86.7 | (100.0) | (100.0) | (100.0) | (100.0) | *** | *** |
| 2. Negative control (50 mM ammonium acetate; pH 8.5) | — | (0.0) | 6.7 | 20.0 | 20.0 | 20.0 | — | — |
| 3. BI45 full strength culture | 2.3 | 13.3 | 13.3 | 20.0 | 26.7 | 33.3 | NS | NS |
| 4. 10X MeOH intracellular extract | 0.44 | 6.7 | 20.0 | 20.0 | 20.0 | 20.0 | NS | NS |
| 5. 10X MeOH-extracted spore pellet | 0.58 | (0.0) | 6.7 | 6.7 | 6.7 | 6.7 | NS | NS |
| 6. 10X Quenched supernatant (un-separated) | 6.0 | 73.3 | 73.3 | 73.3 | 73.3 | 93.3 | ** | *** |
| 7. 10X Quenched supernatant (clear) | 4.6 | 26.7 | 46.7 | 53.3 | 66.7 | 80.0 | * | ** |
| 8. 10X Quenched supernatant (pellet) | 5.4 | (0.0) | 20.0 | 33.3 | 53.3 | 60.0 | NS | * |
| LSD 5% | | 33.68 | 36.55 | 38.4 | 38.56 | 33.69 | | |
| LS Effect 5% (=LSD 5%/√2) | | 23.82 | 25.84 | 27.2 | 27.27 | 23.82 | | |
| LSD 10% | | 27.16 | 29.90 | 31.4 | 31.54 | 27.56 | | |
| LS Effect 10% (=LSD 10%/√2) | | 19.21 | 21.14 | 22.2 | 22.30 | 19.49 | | |
| Overall P-value | | 0.002 | 0.015 | 0.032 | 0.015 | <0.001 | | |

^{a}Significance of difference with control.
NS = Not Significant;
* = P < 0.05;
** = P < 0.01;
*** = P < 0.001.

Table 10 shows the cumulative mortality (%) on days 1 to 5 of the DBM bioassay with Bl45 10× concentrated methanol extracts. Bioassay one of three repeats. Treatments that are constant, 0 or 100, have been omitted from the ANOVA (means are in brackets). To compare two un-bracketed means, the LSD is used. To compare a bracketed mean with an un-bracketed mean, the LS Effect is used.

TABLE 11
Cumulative mortality (%) in the DBM bioassay with BI45 10X concentrated methanol extracts, bioassay two of three repeats.

| | Protein | | | | | | Sig. of dif. | |
|---|---|---|---|---|---|---|---|---|
| | conc. | Cumulative mortality (%) | | | | | with control 2^{a} | |
| Treatment | (mg/ml) | Day 1 | Day 2 | Day 3 | Day 4 | Day 5 | Day 1 | Day 5 |
| 1. Dipel Bt subsp. kurstaki | 0.13 | 83.3 | (100.0) | (100.0) | (100.0) | (100.0) | *** | *** |
| 2. Negative control 1 (50 mM ammonium acetate; pH 8.5) | — | (0.0) | 5.6 | 5.6 | 16.7 | 16.7 | NS | NS |
| 3. Negative control 2 (mLB⁺ and 50 mM ammonium acetate) | — | (0.0) | 5.6 | 11.1 | 27.8 | 27.8 | — | — |
| 4. BI45 full strength culture | 1.4 | 16.7 | 66.7 | 77.8 | 77.8 | 88.9 | * | *** |
| 5. 10X MeOH-extracted spore pellet | 0.31 | (0.0) | (0.0) | (0.0) | (0.0) | 5.6 | NS | * |
| 6. 10X MeOH intracellular extract | 0.20 | 5.6 | 5.6 | 11.1 | 22.2 | 22.2 | NS | NS |
| 7. 10X Quenched supernatant | 6.5 | 77.8 | (100.0) | (100.0) | (100.0) | (100.0) | *** | *** |
| LSD 5% | | 18.38 | 25.44 | 34.64 | 32.77 | 21.42 | | |
| LS Effect 5% (=LSD 5%/√2) | | 12.99 | 17.99 | 24.49 | 23.17 | 15.16 | | |
| LSD 10% | | 14.59 | 20.20 | 27.51 | 26.03 | 17.28 | | |
| LS Effect 10% (=LSD 10%/√2) | | 10.32 | 14.28 | 19.45 | 18.41 | 12.22 | | |
| Overall P-value | | <0.001 | 0.002 | 0.006 | 0.013 | <0.001 | | |

^{a}Significance of difference with control 2.
NS = Not Significant;
* = P < 0.05;
*** = P < 0.001.

Table 11 shows the cumulative mortality (%) on days 1 to 5 of the DBM bioassay with Bl45 10× concentrated methanol extracts, bioassay two of three repeats. Treatments that are constant, 0 or 100, have been omitted from the ANOVA (means are in brackets). To compare two un-bracketed means, the LSD is used. To compare a bracketed mean with an un-bracketed mean, the LS Effect is used.

TABLE 12
Cumulative mortality (%) in the DBM bioassay with BI45 10X concentrated
methanol extracts, bioassay three of three repeats.

| | Protein | | | | | Sig. of dif. | | |
|---|---|---|---|---|---|---|---|---|
| | concentration | Cumulative mortality (%) | | | | with control 3^{a} | | |
| Treatment | (mg/ml) | Day 1 | Day 2 | Day 3 | Day 4 | Day 5 | Day 1 | Day 4 |
| 1. Dipel Bt subsp. kurstaki | 0.13 | 94.4 | (100.0) | (100.0) | (100.0) | (100.0) | *** | *** |
| 2. Negative control 1 (50 mM ammonium | — | (0.0) | (0.0) | 11.1 | 11.1 | 16.7 | NS | NS |
| acetate; pH 8.5) | | | | | | | | |
| 3. Negative control 2 (mLB⁺ and | — | (0.0) | (0.0) | 11.1 | 16.7 | 27.8 | NS | NS |
| 50 mM ammonium acetate) | | | | | | | | |
| 4. Negative control 3 (10X mLB⁺ and | — | (0.0) | (0.0) | 4.8 | 9.5 | 19.0 | — | — |
| 50 mM ammonium acetate) | | | | | | | | |
| 5. BI45 full strength culture | 1.2 | 5.6 | 27.8 | 50.0 | 61.1 | 66.7 | NS | * |
| 6. 10X MeOH-extracted spore pellet | 0.22 | (0.0) | (0.0) | 5.6 | 5.6 | 11.1 | NS | NS |
| 7. 10X MeOH intracellular extract | 0.10 | (0.0) | (0.0) | (0.0) | (0.0) | (0.0) | NS | NS |
| 8. 10X Quenched supernatant | 7.3 | 50.0 | 94.4 | (100.0) | (100.0) | (100.0) | ** | *** |
| LSD 5% | | 30.91 | 39.84 | 35.13 | 36.58 | 38.10 | | |
| LS Effect 5% (=LSD 5%/√2) | | 21.86 | 30.59 | 24.84 | 25.87 | 26.94 | | |
| LSD 10% | | 23.73 | 28.17 | 28.33 | 29.75 | 30.99 | | |
| LS Effect 10% (=LSD 10%/√2) | | 16.78 | 21.63 | 20.03 | 21.04 | 21.91 | | |
| Overall P-value | | 0.003 | 0.013 | 0.081 | 0.047 | 0.051 | | |

^{a}Significance of difference with control 3.
NS = Not Significant;
* = P < 0.05;
** = P < 0.01;
*** = P < 0.001.

Table 12 shows the cumulative mortality (%) on days 1 to 5 of the DBM bioassay with Bl45 10× concentrated methanol extracts, bioassay three of three repeats. Treatments that are constant, 0 or 100, have been omitted from the ANOVA (means are in brackets). To compare two un-bracketed means, the LSD is used. To compare a bracketed mean with an un-bracketed mean, the LS Effect is used.

TABLE 13
Cumulative mortality (%) at day 3 of DBM bioassay repeats 1-3.

| | Cumulative mortality | | | Average cumulative | |
|---|---|---|---|---|---|
| | (%) by day 3 | | | mortality (%) by day 3 | |
| | Bioassay | Bioassay | Bioassay | Bioassays | Bioassays |
| Treatment | 1 | 2 | 3 | 1, 2 & 3 | 2-3 |
| 1. Dipel^{DF} Bt subsp. kustaki 32000 U/mg | (100.0) | (100.0) | (100.0) | (100.0) | (100.0) |
| 2. Negative control 1 (50 mM ammonium acetate; pH 8.5) | 20.0 | 5.6 | 11.1 | 12.2 | 8.3 |
| 3. Negative control 2 (mLB⁺ and 50 mM ammonium acetate) | — | 11.1 | 11.1 | — | 11.1 |
| 4. Negative control 3 (10X mLB⁺ and 50 mM ammonium acetate) | — | — | (0.0) | — | — |
| 5. Bl45 sporulated culture full strength | 20.0 | 77.8 | 50.0 | 49.3 | 63.9 |
| 6. 10X MeOH-extracted spore pellet | 6.7 | (0.0) | 5.6 | 4.1 | 2.8 |
| 7. 10X Intracellular MeOH-extract | 20.0 | 11.1 | (0.0) | 10.4 | 5.6 |
| 8. 10X Quenched supernatant (un-separated) | 73.3 | (100.0) | (100.0) | 91.1 | (100.0) |
| 9. 10X Quenched supernatant (clear fraction) | 53.3 | — | — | — | — |
| 10. 10X Quenched supernatant (pellet fraction) | 33.3 | — | — | — | — |
| LSD 5% | 38.4 | 34.6 | 40.4 | 31.1 | 27.8 |
| LS Effect 5% (=LSD 5%/√2) | 27.2 | 24.5 | 28.6 | 22.0 | 19.7 |
| LSD 10% | 31.4 | 27.5 | 32.1 | 25.1 | 21.4 |
| LS Effect 10% (=LSD 10%/√2) | 22.2 | 19.5 | 22.7 | 17.7 | 15.1 |
| Overall P-value | 0.032 | 0.006 | 0.111 | <0.001 | 0.014 |

P < 0.05 = significant; P < 0.01 = strongly significant; P < 0.001 = highly significant.

Table 13 shows the cumulative mortality (%) at day 3 of DBM bioassay repeats 1-3. Treatments that are constant, 0 or 100, have been omitted from the ANOVA (means are in brackets). To compare two un-bracketed means, the LSD is used. To compare a bracketed mean with an un-bracketed mean, the LS Effect is used.

Additionally, the average protein concentration of the three quenched supernatant samples, used in all three bioassays, was significantly higher than that of the other Bl45 treatments (Table 14). The average protein concentration of the Bl45 full strength cultures was significantly higher than that of the intracellular MeOH-extracts and that of the MeOH-extracted spores, but was significantly lower than that of the quenched supernatant samples (P<0.001). The average protein concentration of the quenched supernatant samples was four times higher than that of the full strength cultures, and was more toxic toward DBM larvae. These results confirmed that the main toxic activity of Bl45 was located in the culture supernatant, and that the toxins were secreted from the cells or spores. The results also suggest that the toxicity was most likely related to the protein content.

SDS-PAGE of the second and third bioassay showed, as found in the first bioassay, two unique bands in the quenched supernatant samples of about 40 and 60 kDa. An additional third band of approximately 50 kDa was identified in the quenched supernatant of the third bioassay.

The 60 kDa band was selected and excised for analysis by Electrospray Ionisation (ESI) mass-spectrometry.

TABLE 14
Protein concentrations of the Bl45concentrated methanol extract bioassay treatments.

| | Protein concentration (mg/ml) | | | |
|---|---|---|---|---|
| | Bioassay | Bioassay | Bioassay | Bioassays |
| Treatment | 1 | 2 | 3 | 1, 2 & 3 |
| 1. Dipel^{DF} Bt subsp. kurstaki | 0.11 | 0.13 | 0.13 | 0.12 |
| 2. Bl45 full strength sporulated culture | 2.30 | 1.40 | 1.20 | 1.63 |
| 3. 10X MeOH-extracted spore pellet | 0.44 | 0.31 | 0.22 | 0.32 |
| 4. 10X Intracellular MeOH-extract | 0.58 | 0.21 | 0.10 | 0.30 |
| 5. 10X Quenched supernatant (not separated) | 6.00 | 6.50 | 7.30 | 6.60 |
| 6. 10X Quenched supernatant (clear fraction) | 4.60 | — | — | — |
| 7. 10X Quenched supernatant (pellet fraction) | 5.40 | — | — | — |
| LSD 5% | — | — | — | 0.847 |
| Overall P-value | — | — | — | <0.001 |

P < 0.05 = significant; P < 0.01 = strongly significant; P < 0.001 = highly significant.

### Example 6. The Identification of a Putative Insecticidal Surface Layer Protein by Electrospray Ionisation Mass Spectrometry

This example presents the identification of an insecticidal protein from B. laterosporus strain NMI No. V12/001945 (Bl45).

The estimated 60 kDa protein band from the highly toxic quenched supernatant was analysed by ESI-mass spectrometry for protein identification.

### Results

The Liquid Chromatography-mass spectrometry graph showed a good fragmentation pattern and signal intensity, which are important criteria for a good sample quality (data not shown).

Database searching of the detected peptides resulted in the identification of a single protein, a surface layer protein from Bl (Table 15). The sequence coverage for the entire protein was low, however, with 1.8% (Table 15).

TABLE 15
NCBI Bacteria (Eubacteria) database search results for the identification of the unknown
putative insecticidal protein from BI45performed with a Mascot v.2.4 server.

| | | | Sequence | | | | Number |
|---|---|---|---|---|---|---|---|
| | Accession | | coverage | | Mw | | of |
| Protein | number | Database | (%) | Score | (kDa) | pI | peptides |
| Surface layer protein | gi\|517499195 | nr_plus* | 1.80 | 237.29 | 121.30 | 5.12 | 2 |
| [B. laterosporus] | | | | | | | |

*Nr = non redundant protein sequences. Description: all non-redundant GenBank CDS translations + PDB + SwissProt + PIR + PRF excluding environmental samples from WGS projects.

The estimated protein mass of this protein was approximately 121 kDa. Two unique peptides were used for the detection of this protein (Table 16), indicating that the peptides were not related to any other proteins in the database.

TABLE 16
ESI-mass spectrometry identified peptides used for the identification of the unknown
putative insecticidal protein from Brevibacillus laterosporus 1951.

| | No. | m/z | Δ m/z | | Rt | | | | | |
|---|---|---|---|---|---|---|---|---|---|---|
| Cmpd | Cmpds | meas. | (ppm) * | z | [min] | Score * | P | Range | Sequence | Type |
| 1239 | 3 | 601.8450 | 3.97 | 2 | 27.46 | 47.91 | 0 | 179-189 | R.ALGYEPLALQK.G | CID |
| 634 | 4 | 490.7310 | −50.96 | 2 | 22.86 | 45.50 | 0 | 442-449 | R.YLSIVDDR.S | CID |

* Criteria for accurate peptide identification by ESI-mass spectrometry:
Δ m/z (ppm) should lie in between −150 and 150.
Score for peptide identification should be >30.

The Δ m/z for both peptides lay within the criterion for an accurate m/z measurement, which lies in between −150 and 150 (Table 16). Additionally, the corresponding scores of both peptide identifications lay within the criterion for a significant peptide identification, which is above 30 (Table 16). These results support the identification of this Bl surface layer protein as the protein of interest.

One of the two peptides was used to identify the gene that encodes the surface layer protein within the Bl45 genome using Geneious version 8.1.7. The gene was identified and found in contig 541 (FIG. 10 A). The peptide sequence, RALGYEPLALQKG, was found to be within frame with the predicted ORF of the gene of interest, reverse frame 1. The ORF has 3318 nucleotide residues and a protein translation of 1105 residues with an estimated protein mass of a 124 kDa (Protein Molecular Weight, 2016).

The identified gene was annotated using the Basic Local Alignment Search Tool x (BLASTx) in the NCBI non-redundant protein database (nr). The database search resulted in the identification of a surface layer protein of Bl as well, with low E-values of 0 for the first 21 hits, and very low E-values for hits 22 to 39, indicating the high significance of these matches (Table 17). Thirty-seven of the 39 significant hits belonged to the genus Brevibacillus, with the seven significantly highest matches belonging to the Bl species. The two highest matches had 98% query coverage and 82% identity scores. These results demonstrate that the S-layer protein of Bl45 is highly conserved within the Bl species and the Brevibacillus genus, but differs for at least 18% compared to the most related proteins in the NCBI Bacteria and Archaea database.

TABLE 17
Significant alignment scores (query coverage >200) of BLASTx search results of the putative accessory
virulent cell wall binding protein-encoding gene. Genetic code used, Bacteria and Archaea (NCBI).

| | | Query | | | |
|---|---|---|---|---|---|
| No. | Description | cover | Identity | E-value | Accession |
| 1 | Surface layer protein, Brevibacillus laterosporus | 98% | 82% | 0 | WP_022586171.1 |
| 2 | Cell surface protein, Brevibacillus sp. SKDU10 | 98% | 82% | 0 | WP_064016380.1 |
| 3 | Surface layer protein, Brevibacillus laterosporus | 95% | 79% | 0 | WP_018669772.1 |
| 4 | Cell surface protein, Brevibacillus laterosporus | 95% | 78% | 0 | WP_041752424.1 |
| 5 | Cell surface protein, Brevibacillus laterosporus | 95% | 79% | 0 | WP_068792156.1 |
| 6 | Cell surface protein, Brevibacillus laterosporus | 95% | 78% | 0 | WP_031412936.1 |
| 7 | Surface layer protein, Brevibacillus laterosporus | 95% | 77% | 0 | WP_003342361.1 |
| 8 | Middle cell wall protein precursor, Brevibacillus agri BAB-2500 | 74% | 46% | 0 | ELK41453.1 |
| 9 | Middle cell wall protein, Brevibacillus reuszeri | 74% | 45% | 0 | WP_049740903.1 |
| 10 | Putative S-layer protein, Brevibacillus sp. BC25 | 74% | 46% | 0 | EJL27528.1 |
| 11 | S-layer protein, Brevibacillus sp. CF112 | 68% | 45% | 0 | WP_007778484.1 |
| 12 | Middle cell wall protein, Brevibacillus agri | 68% | 45% | 0 | WP_039971291.1 |
| 13 | Middle cell wall protein, Brevibacillus agri | 68% | 45% | 0 | WP_025843645.1 |
| 14 | Middle cell wall protein, Bacillus sp. NSP2.1 | 68% | 45% | 0 | WP_026557553.1 |
| 15 | Middle cell wall protein, Brevibacillus panacihumi W25 | 82% | 44% | 0 | EST55341.1 |
| 16 | RecName: Full = Surface layer protein; AltName: Full = | 74% | 44% | 0 | P38538.1 |
| | Hexagonal wall protein; Short = HWP; Flags: Precursor | | | | |
| 17 | Middle cell wall protein, Brevibacillus reuszeri | 68% | 45% | 0 | KNB72116.1 |
| 18 | Middle cell wall protein, Brevibacillus choshinensis | 68% | 45% | 0 | WP_055745651.1 |
| 19 | Middle cell wall protein, Brevibacillus parabrevis | 68% | 45% | 0 | WP_063229029.1 |
| 20 | Middle cell wall protein, Brevibacillus sp. BC25 | 68% | 46% | 0 | WP_039960718.1 |
| 21 | Middle cell wall protein, Brevibacillus thermoruber | 68% | 46% | 0 | WP_035296055.1 |
| 22 | Middle cell wall protein, Brevibacillus borstelensis | 68% | 45% | 1.00E−178 | WP_024983778.1 |
| 23 | Middle cell wall protein, Brevibacillus brevis | 68% | 45% | 3.00E−178 | WP_015893589.1 |
| 24 | Middle wall protein precursor, Brevibacillus brevis | 68% | 45% | 3.00E−178 | AAA22760.1 |
| 25 | Middle cell wall protein precursor, Brevibacillus borstelensis | 68% | 45% | 4.00E−178 | WP_003388368.1 |
| 26 | Middle cell wall protein, Brevibacillus brevis | 68% | 45% | 6.00E−178 | WP_064200427.1 |
| 27 | Middle cell wall protein, Brevibacillus panacihumi | 76% | 43% | 2.00E−177 | WP_031306290.1 |
| 28 | Middle cell wall protein, Brevibacillus formosus | 67% | 45% | 1.00E−175 | WP_047069781.1 |
| 29 | MULTISPECIES: Middle cell wall protein, Bacillales | 68% | 44% | 3.00E−174 | WP_016740216.1 |
| 30 | Hypothetical protein, Brevibacillus massiliensis | 77% | 42% | 8.00E−174 | WP_019121115.1 |
| 31 | Middle cell wall protein, Brevibacillus thermoruber | 68% | 44% | 4.00E−173 | WP_029100336.1 |
| 32 | Middle cell wall protein, Aeribacillus pallidus | 68% | 44% | 1.00E−172 | WP_044898600.1 |
| 33 | Middle cell wall protein, Brevibacillus borstelensis | 69% | 44% | 3.00E−172 | WP_031932663.1 |
| 34 | Middle cell wall protein, Brevibacillus brevis | 67% | 45% | 2.00E−171 | WP_069850953.1 |
| 35 | Middle cell wall protein, Brevibacillus brevis | 67% | 44% | 6.00E−170 | WP_017252324.1 |
| 36 | Middle cell wall protein, Brevibacillus sp. WF146 | 68% | 44% | 3.00E−168 | WP_065067417.1 |
| 37 | MULTISPECIES: Middle cell wall protein, Brevibacillus | 67% | 44% | 1.00E−167 | WP_048035058.1 |
| 38 | Middle wall protein precursor, Brevibacillus brevis | 15% | 61% | 2.00E−60 | AAA87321.1 |
| 39 | Hypothetical protein, Tepidibacillus decaturensis | 46% | 30% | 2.00E−50 | WP_068726325.1 |

Two surface layer homology (SLH) domains and a 235 kDa rhoptry family protein domain were identified as putative conserved domains within the S-layer protein of Bl45 with BLASTx and Domain Enhanced Lookup Time Accelerated (DELTA) BLAST, respectively (FIGS. 10 B and 10 C, Table 18).

TABLE 18
Putative conserved domains detected in the surface layer-
encoding gene of Bl45by Blastx and DELTA-Blast (NCBI).

| | Family | | Nucleotide | | |
|---|---|---|---|---|---|
| No. | domain name | Description | interval | E-value | Accession |
| 1 | SLH | Surface Layer Homology domain | 89-124 | 4.15e−4 | Pfam00395 |
| 2 | SLH | Surface Layer Homology domain | 147-188 | 3.19e−9 | Pfam00395 |
| 3 | 235 KDa | Reticulocyte binding rhoptry | 757-1527 | 9.97e−3 | TIGR01612 |
| | rhoptry protein | protein from Plasmodium yoelii | | | |
| | | yoelli 17XNL | | | |

The S-layer protein sequence was analysed by tBLASTn, from amino acid to nucleotide, to determine the degree of gene conservation with other bacteria. The results showed that the gene was conserved in two Bl strains, LMG 15441 and B9, with the lowest E-value of 0.0 (Table 19). The query cover of both matches was 96% and the gene identities were 72% and 71% for Bl LMG 15441 and Bl B9, respectively. This demonstrates that the Bl45 S-layer encoding gene is conserved within these other Bl strains, but differs at least 28% from the conserved genes from Bl LMG 15441 and Bl B9. The gene is also well conserved in Brevibacillus brevis, but the query cover and gene identities were considerably lower than the Bl matches. The E-values were very low however, from 0.0 to 1.0×10⁻⁷², indicating the high significance of these matches.

TABLE 19
Significant alignment scores (query coverage is >200) of a tBLASTn search,
from protein to nucleotide, of the putative surface layer protein-encoding gene
from Brevibacillus laterosporus 1951. Nucleotide database nr/nt (NCBI).

| | | Query | | E- | |
|---|---|---|---|---|---|
| No. | Gene hit | cover | Identity | value | Accession |
| 1 | Brevibacillus laterosporus LMG 15441 complete genome | 96% | 72% | 0.0 | CP007806.1 |
| 2 | Brevibacillus laterosporus B9 complete genome | 96% | 71% | 0.0 | CP011074.1 |
| 3 | Brevibacillus brevis HWP gene for cell wall protein, | 75% | 42% | 0.0 | D90050.1 |
| | complete cds | | | | |
| 4 | Brevibacillus brevis NBRC 100599, complete genome | 75% | 44% | 0.0 | AP008955.1 |
| 5 | Brevibacillus brevis outer wall protein, 5′ end, and | 75% | 44% | 0.0 | M19115.1 |
| | middle wall protein, complete cds | | | | |
| 6 | Bacillus brevis 47, cell wall protein gene operon, 5′ region | 22% | 54% | 1e−72 | M15464.1 |

The amino acid sequence of the S-layer protein from Bl45 was searched against a database of structurally elucidated proteins. Homology to two structurally mapped proteins was detected (FIG. 11). The first homology was to the SLH-domain of the Sap S-layer protein from Bacillus anthracis, with a query cover of 14% and 23%. The second homology was to the cellobiohydrolase enzyme of Clostridium thermocellum, with a query cover of 3% and 23% identity. The third homology was to a chitinase of Paenibacillus sp., with a query cover of 8% and 16% identity (Table 20).

TABLE 20
Swiss-model analysis of putative surface layer amino acid
sequence from Brevibacillus laterosporus 1951.

| | | | Query | | |
|---|---|---|---|---|---|
| No. | Protein | Description | cover | Identity | Reference |
| 1 | Sap | Surface layer protein from Bacillus anthracis. | 14% | 23% | (Kern et al., 2011) |
| 2 | ChiW | Chitinase from Paenibacillus sp. str FPU-7 | 8% | 16% | (Itoh et al., 2016) |
| 3 | CelS | Cellobiohydrolase from cellulosome of Clostridium | 3% | 29% | (Guimaraes et al., |
| | | thermocellum. | | | 2002) |

In summary, the putative toxin protein band, derived from a highly DBM lethal quenched supernatant, was identified as a S-layer protein that is conserved in Bl and the Brevibacillus genus. The S-layer protein is predicted to have two SLH-domains, and a low degree of homology to a 235 kDa rhoptry family domain.

### Example 7. Identification of Putative Accessory Virulent Genes

This example describes the analysis of two genes adjacent the S-layer protein gene identified above. These were of interest as adjacent genes can have related or similar functions, and/or in this case may be part of the apparatus that processes toxins or otherwise contribute to the function of toxin encoding genes.

### Adhesin-Like Encoding Gene

The first ORF located upstream from the S-layer encoding gene was annotated as a putative adhesion-encoding gene (FIG. 12, left ORF). The ORF has 2577 nucleotides and the translation results in 858 amino acids with a predicted molecular mass of 89 kDa. The BLASTx NCBI non-redundant protein (nr) database search resulted in the identification of a hypothetical protein for the highest four protein homologs (Table 21). The query cover and sequence identity of the most significant homolog, a hypothetical protein of Bacillus manliponensis, were 86% and 41% respectively, with an E-value of 1×10⁻¹¹⁸.

TABLE 21
Significant alignment scores (query coverage >200) of BLASTx search results of putative accessory
virulent cell-wall binding protein encoding gene. Genetic code used, Bacteria and Achaea.

| | | Query | | | |
|---|---|---|---|---|---|
| No. | Name | cover | Identity | E-value | Accession |
| 1 | Hypothetical protein, Bacillus | 86% | 41% | 1.00E−118 | WP_034639476.1 |
| | manliponensis | | | | |
| 2 | Hypothetical protein, Brevibacillus sp. | 78% | 41% | 5.00E−113 | WP_056486945.1 |
| | Leaf182 | | | | |
| 3 | Hypothetical protein, Viridibacillus arenosi | 85% | 40% | 2.00E−107 | WP_038179777.1 |
| 4 | Hypothetical protein, Desulfotomaculum | 69% | 42% | 3.00E−107 | WP_066670535.1 |
| | sp. LMa1 | | | | |
| 5 | Cell wall-binding repeat 2 family protein, | 83% | 40% | 6.00E−103 | WP_049040859.1 |
| | Clostridium sporogenes | | | | |
| 6 | Hypothetical protein, Bacillus | 68% | 44% | 2.00E−98 | WP_019243132.1 |
| | massilioanorexius | | | | |
| 7 | Hypothetical protein Lysinibacillus | 69% | 40% | 1.00E−97 | WP_053585327.1 |
| | contaminans | | | | |
| 8 | Hypothetical protein Bacillus sp. NSP2.1 | 76% | 37% | 2.00E−89 | WP_026557552.1 |
| 9 | Hypothetical protein Exiguobacterium sp. | 67% | 37% | 1.00E−85 | WP_023469467.1 |
| | MH3 | | | | |
| 10 | Cell wall-binding repeat 2 family protein, | 70% | 38% | 5.00E−81 | WP_045886825.1 |
| | Clostridium botulinum | | | | |
| 11 | MULTISPECIES: cell wall-binding Repeat 2 | 70% | 38% | 2.00E−80 | WP_040109012.1 |
| | family protein, Clostridium | | | | |
| 12 | hypothetical protein, Paenibacillus | 63% | 38% | 2.00E−80 | WP_025702545.1 |
| | forsythiae | | | | |
| 13 | Hypothetical protein, Fictibacillus sp. FJAT- | 80% | 35% | 2.00E−76 | WP_062237590.1 |
| | 27399 | | | | |
| 14 | Hypothetical protein Paenibacillus wynnii | 65% | 37% | 2.00E−76 | WP_052087943.1 |
| 15 | S-layer, Viridibacillus arvi | 64% | 39% | 4.00E−71 | CRX56286.1 |
| 16 | Hypothetical protein, Bacillus acidicola | 67% | 35% | 2.00E−67 | WP_066269135.1 |
| 17 | S-layer protein, Anoxybacillus | 63% | 38% | 4.00E−63 | WP_052023532.1 |
| | flavithermus | | | | |
| 18 | S-layer domain-containing protein, | 63% | 38% | 4.00E−63 | GAC90612.1 |
| | Anoxybacillus flavithermus NBRC 109594 | | | | |
| 19 | Hypothetical protein, Brevibacillus sp. | 16% | 77% | 3.00E−60 | WP_064016379.1 |
| | SKDU10 | | | | |

The low E-value indicates a high significance of the match. However, the homolog still differs over 50% from the protein from Bl45. Additionally, merely three of the twenty significant homologs detected, homologs 2, 19 and 20, belonged to the Brevibacillus (Table 21). These results suggest that the putative adhesion-like protein from Bl45 is not highly conserved within the Brevibacillus species in the NCBI non-redundant protein databank.

The level of gene conservation of the potential adhesin-like protein was analysed using tBLASTn, from protein to translated nucleotide. The database search yielded three significant gene homologs derived from Exiguobacterium sp., Clostridium botulinum and Viridibacillus arvi, respectively (Table 22). The best matching gene homolog from Exiguobacterium sp. had a query cover of 67% and a sequence identity of 36%, with an E-value of 7×10⁻⁸³. The low E-value indicates a considerable significance of the match, but the sequence identity was merely 36%, suggesting that the Bl45 putative adhesion-like gene is not highly conserved in other bacterial species, in particular Brevibacillus species, in the NCBI gene databank.

TABLE 22
Significant alignment scores (query coverage is >200)
of a tBLASTn search, from protein to nucleotide, of
the putative adhesin encoding gene from Brevibacillus laterosporus
1951. Nucleotide database nr/nt (NCBI).

| | | Query | Sequence | E- | |
|---|---|---|---|---|---|
| No. | Gene hit | cover | identity | value | Accession |
| 1 | Exiguobacterium sp. | 67% | 36% | 7e−83 | CP006866.1 |
| | MH3 complete | | | | |
| | genome | | | | |
| 2 | Clostridium | 70% | 36% | 2e−79 | CP006902.1 |
| | botulinum | | | | |
| | Prevot_594 genome | | | | |
| 3 | Viridibacillus | 64% | 37% | 2e−70 | LN867317.1 |
| | arvi JGB58 slp2 | | | | |
| | gene for S-layer | | | | |

Potential functional domains were identified using BLASTx and DELTA-BLAST. The BLASTx database search yielded three potential functional domains, and the DELTA-BLAST database search yielded two potential functional domains (FIG. 13 and Table 23). Four of the five identified domains were related to one another; the flagellin and flagellar hook associated protein domains, and the fimbrillin-like and autotransporter adhesin domains (Table 23). The E-values of these matches were relatively high with 1.1×10⁻³ and 5.6×10⁻³ for the flagellin and flagellar hook associated protein domains, respectively, and with 2.2×10⁻³ and 8.3×10⁻³ for the fimbrillin-like and autotransporter adhesin domains, respectively. The domain of unknown function had an E-value of 0.01. These relatively high E-values demonstrate the relatively low significance of the matches but provide an insight however, into the potential function of the seemingly novel adhesin-like encoding gene from Bl45.

TABLE 23
Putative conserved domains in the putative virulent cell wall binding
protein encoding gene identified by Blastx and DELTA-Blast (NCBI).

| | Family | | | | |
|---|---|---|---|---|---|
| | domain | | | | |
| No. | name | Description | Interval | E-value | Accession |
| 1 | PRK12806 | Flagellin; Provisional. | 1078-1959 | 1.06E−03 | PRK12806 |
| 2 | Mfa_like_1 | Fimbrillin-like. Part of FimbA (CLD450) | 1441-1908 | 2.18E−03 | pfam13149 |
| | | superfamily of adhesin components or | | | |
| | | fimbrillins. | | | |
| 3 | Hia | Autotransporter adhesin. | 1675-2436 | 8.27E−03 | COG5295 |
| 4 | DUF4815 | Domain of unknown function (DUF4815). | 724-834 | 0.01 | pfam16075 |
| 5 | flgK | Flagellar hook-associated protein; Validated. | 733-811 | 5.56E−03 | PRK08147 |

The translated amino acid sequence was assessed against a database of structurally elucidated proteins using SWISS-MODEL. The database analysis yielded five different structurally elucidated protein homologs (FIG. 14 and Table 24). Three of the five homologs, SiE from Salmonella enterica, MpAFP from Marinomonas primoryensi and CIfB from Staphylococcus aureus, are adhesin proteins, of which SiE and CIfB are important virulence factors that provide the colonisation of the host cells. The SbsC is an S-layer protein from Geobacillus stearothermophilus to which the adhesin-like protein from Bl45 had the highest homology.

TABLE 24
Swiss-model analysis of putative fimbriae/adhesin amino
acid sequence from Brevibacillus laterosporus 1951.

| | | | Query | | |
|---|---|---|---|---|---|
| No. | Protein | Description | cover | Identity | Reference |
| 1 | SbsC | Surface layer protein SbsC from Geobacillus | 23 | 21 | (Pavkov et al., |
| | | stearothermophilus | | | 2008) |
| 2 | SiiE | Large calcium binding adhesin of Salmonella | 17 | 19 | (Griessl et al., |
| | | enterica | | | 2013) |
| 3 | SbsC | Surface layer protein SbsC from Geobacillus | 16 | 25 | (Pavkov et al., |
| | | stearothermophilus | | | 2008) |
| 4 | SbsC | Surface layer protein SbsC from Geobacillus | 16 | 14 | (Pavkov et al., |
| | | stearothermophilus | | | 2008) |
| 5 | MpAFP | Ice-binding adhesin MpAFP from Marinomonas | 6 | 24 | (Guo et al., 2013) |
| | | primoryensis | | | |
| 6 | CopC | Copper binding protein CopC from | 8 | 11 | (Arnesano et al., |
| | | Pseudomonas syringae | | | 2003) |
| 7 | ClfB | Anti clumping factor ClfB from Staphylococcus | 7 | 18 | (Ganesh et al., in |
| | | aureus | | | prep.) |

This analysis suggests that this adhesin-like protein from Bl45 may itself be an S-layer protein. The CopC protein from the phytopathogen Pseudomonas syringe is an important virulence factor, but is not of adhesive nature. It has a very low query cover and sequence identity to the adhesin-like protein from Bl45 with 8% and 11%, respectively. The query cover and sequence identity of the best matching homolog, SbsC, were merely 23% and 21%, demonstrating the low significance of these matches. The homologs may, however provide, together with the identified potential functional domains, an indication as to the function of the adhesin-like protein from Bl45.

In summary, these results suggest that the gene located upstream from the S-layer protein encoding gene may be an adhesin-like protein that facilitates adherence to the insect host cells, and could therefore be an important virulence factor of Bl45.

### Efflux Pump Encoding Gene

The second ORF located downstream from the S-layer protein-encoding gene was annotated as an efflux pump protein (FIG. 12, right ORF). The ORF has 1197 nucleotides and 398 amino acids with a predicted molecular mass of 44 kDa. The BLASTx nr database search resulted in the identification of 41 significant protein homologs of which 90% belongs to the Brevibacillus (Table 25). The top seven scoring protein homologs were transporter proteins belonging to Bl and have the lowest possible E-value of 0.0, demonstrating the highest degree of significance for these matches. The query cover of all seven homologs was 97% and the sequence identity varies from 82% to 84%, indicating a high degree of conservation of the transporter-encoding gene within Bl.

TABLE 25
Significant alignment scores (query coverage >200) of BLASTx search results of putative
accessory virulent transporter encoding gene. Genetic code used, Bacteria and Archaea.

| | | Query | | | |
|---|---|---|---|---|---|
| No. | Description | cover | Identity | E-value | Accession |
| 1 | Transporter, | 97% | 84% | 0.0 | WP_018669771.1 |
| | Brevibacillus laterosporus | | | | |
| 2 | Transporter, | 97% | 84% | 0.0 | WP_031412935.1 |
| | Brevibacillus laterosporus | | | | |
| 3 | Transporter | 97% | 82% | 0.0 | WP_003342359.1 |
| | Brevibacillus laterosporus | | | | |
| 4 | Transporter, | 97% | 83% | 0.0 | WP_064016381.1 |
| | Brevibacillus sp. SKDU10 | | | | |
| 5 | Transporter, | 97% | 82% | 0.0 | WP_068792157.1 |
| | Brevibacillus laterosporus | | | | |
| 6 | Transporter, | 97% | 82% | 0.0 | WP_022586170.1 |
| | Brevibacillus laterosporus | | | | |
| 7 | Transporter, | 97% | 82% | 0.0 | WP_003334287.1 |
| | Brevibacillus laterosporus | | | | |
| 8 | Hypothetical protein, Brevibacillus | 95% | 47% | 2.00E−97 | WP_019121114.1 |
| | massiliensis | | | | |
| 9 | Hypothetical protein, Brevibacillus brevis | 95% | 42% | 5.00E−83 | WP_064200426.1 |
| 10 | Membrane protein, | 95% | 42% | 2.00E−82 | WP_007722047.1 |
| | Brevibacillus sp. BC25 | | | | |
| 11 | Membrane protein, | 95% | 42% | 3.00E−82 | WP_025843646.1 |
| | Brevibacillus agri | | | | |
| 12 | Hypothetical protein, Brevibacillus agri | 95% | 42% | 3.00E−82 | WP_005831828.1 |
| 13 | Membrane protein, | 95% | 42% | 4.00E−82 | WP_041749690.1 |
| | Brevibacillus brevis | | | | |
| 14 | Membrane protein, | 95% | 42% | 1.00E−81 | WP_047069782.1 |
| | Brevibacillus formosus | | | | |
| 15 | Hypothetical protein, Brevibacillus | 95% | 41% | 1.00E−81 | WP_063229028.1 |
| | parabrevis | | | | |
| 16 | MULTISPECIES: membrane protein, | 95% | 41% | 2.00E−81 | WP_007778481.1 |
| | Bacillales | | | | |
| 17 | MULTISPECIES: membrane protein, | 95% | 42% | 7.00E−81 | WP_016740215.1 |
| | Bacillales | | | | |
| 18 | MULTISPECIES: membrane Protein, | 95% | 43% | 2.00E−80 | WP_048035059.1 |
| | Brevibacillus | | | | |
| 19 | Membrane protein, Brevibacillus | 95% | 41% | 2.00E−80 | WP_023555234.1 |
| | panacihumi | | | | |
| 20 | Membrane protein, | 95% | 43% | 4.00E−79 | WP_035296057.1 |
| | Brevibacillus thermoruber | | | | |
| 21 | Hypothetical protein, Brevibacillus brevis | 95% | 43% | 4.00E−79 | WP_069850956.1 |
| 22 | Membrane protein, Brevibacillus brevis | 95% | 43% | 8.00E−79 | WP_017252325.1 |
| 23 | Membrane protein, Brevibacillus | 95% | 42% | 2.00E−78 | WP_029100337.1 |
| | thermoruber | | | | |
| 24 | Hypothetical protein, Brevibacillus sp. | 95% | 42% | 4.00E−77 | WP_065067418.1 |
| | WF146 | | | | |
| 25 | Membrane protein, | 95% | 42% | 5.00E−77 | WP_044898601.1 |
| | Aeribacillus pallidus | | | | |
| 26 | Transporter, | 96% | 42% | 1.00E−76 | WP_064017674.1 |
| | Brevibacillus sp. SKDU10 | | | | |
| 27 | Transporter, | 96% | 42% | 1.00E−76 | WP_031411156.1 |
| | Brevibacillus laterosporus | | | | |
| 28 | Transporter, | 91% | 43% | 3.00E−75 | WP_018671476.1 |
| | Brevibacillus laterosporus | | | | |
| 29 | Hypothetical protein, Brevibacillus | 82% | 44% | 1.00E−74 | WP_019119310.1 |
| | massiliensis | | | | |
| 30 | Hypothetical protein BBR47_54170, | 93% | 41% | 3.00E−74 | BAH46394.1 |
| | Brevibacillus brevis NBRC 100599 | | | | |
| 31 | Transporter, | 95% | 42% | 6.00E−74 | WP_003339158.1 |
| | Brevibacillus laterosporus | | | | |
| 32 | Hypothetical protein, Brevibacillus | 95% | 40% | 7.00E−74 | WP_049740902.1 |
| | reuszeri | | | | |
| 33 | Transporter, | 95% | 42% | 1.00E−73 | WP_003335276.1 |
| | Brevibacillus laterosporus | | | | |
| 34 | Transporter, | 95% | 41% | 2.00E−73 | WP_022583785.1 |
| | Brevibacillus laterosporus | | | | |
| 35 | Transporter, | 96% | 41% | 6.00E−73 | WP_068792740.1 |
| | Brevibacillus laterosporus | | | | |
| 36 | Hypothetical protein, Brevibacillus | 83% | 41% | 2.00E−72 | WP_055745652.1 |
| | choshinensis | | | | |
| 37 | Membrane protein, | 95% | 36% | 3.00E−67 | WP_024983777.1 |
| | Brevibacillus borstelensis | | | | |
| 38 | Hypothetical protein, Brevibacillus | 95% | 36% | 5.00E−67 | WP_003388369.1 |
| | borstelensis | | | | |
| 39 | Membrane protein, | 83% | 40% | 9.00E−63 | KKX54733.1 |
| | Brevibacillus borstelensis cifa_chp40 | | | | |
| 40 | Membrane protein, | 83% | 40% | 9.00E−63 | WP_037192976.1 |
| | Rhodococcus rhodochrous | | | | |
| 41 | Hypothetical protein, Brevibacillus | 79% | 41% | 9.00E−60 | WP_051925512.1 |
| | borstelensis | | | | |

The degree of gene conservation was analysed using tBLASTn, protein to translated nucleotide. The database search generated three significant gene homologs (Table 26). The highest two ranking homologs belong to the Bl species B9 and LMG 15441, respectively.

The Bl B9 gene homolog had a query cover of a 100% and 78% sequence identity. The Bl LMG 15441 gene homolog had a query cover of 99% and 78% sequence identity. Both E-values were zero, illustrating the high significance of these matches. The third ranking gene homolog belonged to Brevibacillus brevis and had a query cover of 97% and sequence identity of 39%, with an E-value of 5×10⁻⁷⁶. These results show how well conserved the putative transporter encoding gene from Bl45 is within Bl genomes deposited in the NCBI gene databank. The gene appears to be conserved in the rest of the Brevibacillus genus as well, but to a lower degree.

TABLE 26
Significant alignment scores (query coverage is >200) of a tBLASTn search,
from protein to nucleotide, of the putative virulent cell wall binding encoding
gene from Brevibacillus laterosporus 1951. Nucleotide database nr/nt (NCBI).

| | | Query | Sequence | | |
|---|---|---|---|---|---|
| No. | Gene hit | cover | identity | E-value | Accession |
| 1 | Brevibacillus laterosporus B9, complete genome | 100% | 79% | 0.0 | CP011074.1 |
| 2 | Brevibacillus laterosporus LMG 15441, complete | 99% | 78% | 0.0 | CP007806.1 |
| | genome | | | | |
| 3 | Brevibacillus brevis NBRC 100599, complete | 97% | 39% | 5e−76 | AP8955.1 |
| | genome | | | | |

Potential functional domains were identified using BLASTx and DELTA-BLAST. Both database searches identified the same functional domains, the outer membrane efflux protein (OEP) superfamily and the outer membrane protein TolC multi domain (FIG. 15 and Table 27). BLASTx detected one OEP superfamily domain and one TolC multi domain, with the latter domain covering 90% of the entire query sequence. The superfamily domain covered 19% of the query sequence and was located within the TolC multi domain. Both domains had relatively low E-values of 2.92×10⁻¹² and 2.4×10⁻⁸ for TolC and OEP domains, respectively, indicating the considerable significance of these two domain hits. The TolC multi domain detected by DELTA-BLAST covered 93% of the entire query sequence and had an E-value of 2.9×10⁻²³, demonstrating a relatively high significance. Two instead of one OEP superfamily domains were detected by DELTA-BLAST, with both domains collectively covering 51% of the entire query sequence and located within the TolC multi domain. The E-values of both OEP superfamily domains were relatively low with 7.8×10⁻¹⁰ and 1.3×10⁻⁶, respectively, exhibiting a considerable significance. These results represent a high confidence level that the Bl45 putative transporter query sequence belongs to the same protein families as the sequences used to create the OEP superfamily and the TolC multi-domain models.

TABLE 27
Putative conserved domains in the putative virulent transporter protein
encoding gene identified by Blastx and DELTA-Blast (NCBI).

| | Family | | | | |
|---|---|---|---|---|---|
| | domain | | | | |
| No. | name | Description | Interval | E-value | Accession |
| 1 | OEP | Outer membrane efflux protein; The OEP family | 874-1107 | 2.44E−08 | pfam02321 |
| 2 | TolC | Outer membrane protein; TolC | 94-1173 | 2.92E−12 | COG1538 |
| 3 | OEP* | Outer membrane efflux protein; The OEP family | 290-391 | 7.84E−10 | pfam02321 |
| 4 | OEP* | Outer membrane efflux protein; The OEP family | 123-224 | 1.33E−06 | pfam02321 |
| 5 | TolC* | Outer membrane protein; TolC | 20-391 | 2.86E−23 | COG1538 |

*Detected by DELTA-BLAST.

The translated amino acid sequence was assessed against a database of structurally elucidated proteins using SWISS-MODEL. The database analysis yielded four structurally elucidated protein homologs (FIG. 16 and Table 28). All protein homologs were outer membrane efflux pumps from human pathogens; ST50 OEP from Salmonella enterica typhi, CusC OEP from Escherichia coli, TolC from E. coli and OprN from Pseudomonas aeruginosa. The two highest-ranking protein homologs, ST50 from S. enterica typhi and CusC from E. coli, both had a high query coverage of 82% and 83%, respectively. The sequence identities of both protein homologs were only 20% and 19%, however, indicating that the putative transporter protein from Bl45 is markedly different from the structurally elucidated OEP homologs. However, taking the identified OEP superfamily, the TolC multi domain and structurally elucidated OEP homologs of the Bl45 putative transporter query sequence into consideration, convincingly suggests that the putative transporter is an outer membrane efflux protein.

TABLE 28
Swiss-model analysis of putative outer membrane efflux protein
amino acid sequence from Brevibacillus laterosporus 1951.

| | | | Query | | |
|---|---|---|---|---|---|
| No. | Protein | Description | cover | Identity | Reference |
| 1 | ST50 | Outer membrane efflux protein ST50, from | 82% | 20% | (Guan et al., 2015) |
| | | Salmonella enterica typhi | | | |
| 2 | CusC | Outer membrane efflux protein component | 83% | 19% | (Lei et al., 2014) |
| | | CusC from Escherichia coli | | | |
| 3 | TolC | Outer membrane efflux protein TolC from | 42% | 15% | (Bavro et al., |
| | | Escherichia coli | | | 2008) |
| 4 | OprN | Outer membrane factor of multidrug | 38% | 13% | (Yonehara et al., |
| | | tripartite efflux pump from Pseudomonas | | | 2016) |
| | | aeruginosa | | | |
| 5 | CusC | Outer membrane efflux protein component | 38% | 13% | (Lei et al., 2014) |
| | | from CusC Escherichia coli | | | |

### Example 8: Characterisation of Insecticidal Activity

This example presents further experiments to characterise an insecticidal activity from Brevibacillus laterosporus strain NMI No. V12/001945 (Bl45).

### Methods

### Time-Course Cell Culture

Cell cultures of Bl45 were inoculated with freshly streaked single colony and incubated at 30° C., 220 rpm in 100 mL nutrient broth (1% glucose pH 7.0). Aliquots of samples harvested at Time=0 hr, 6 hr, 23 hr, 30 hr, 46 hr, 72 hr and 96 hr were taken. Abs₆₀₀, spore counts and cell morphology were examined and samples were stored at −80° C. for future use.

### Protein Expression Profiles

Time-course samples were analysed on a 4-12% NuPAGE Bis-Tris mini gel under reducing conditions. Samples were prepared in LDS sample buffer with reducing agent (DTT) according to manufacturer's instructions. Prior to loading, all samples were heat-denatured at 70° C. for 10 min. MES running buffer was used with NuPAGE anti-oxidant added in the upper buffer chamber to maintain a constant reducing condition throughout the electrophoresis process. Gel was run at a constant voltage setting at 200V, with variable current/power settings at ˜160 mA and ˜35 W respectively, for approx. 30-45 min. After electrophoresis the gel was stained in Coomassie Blue shaking at RT for overnight, then destained in destain solution (40% EtOH, 10% acetic acid) until background was clear. The gel was then kept in water to remove any remaining destain solution on the gel surface, followed by drying between two thin sheets of cellophane for long term storage.

### Bioassay—Heated Vs Unheated Time-Course Biomass Pellet Samples

Cell cultures at 30 hr, 72 hr and 96 hr were harvested and washed as described previously. Briefly, harvested cell cultures were centrifuged at 8000 rpm, 4° C. for 15 min to separate the bacterial biomass (pellet) and growth medium (supernatant). Pellet samples were resuspended in 10 mL MQ-H₂O and mixed thoroughly by vortex, followed by another round of centrifugation under the same condition for 10 min. A total of 6 washes/spins were performed including the initial spin. For each time-course sample, half the amount of washed pellet was heated at 100° C. for 30 min, while the remaining half untreated. All samples were then analysed by SDS-PAGE prior to DBM larvae leaf-dipping bioassay to check for bioactivity. The leaf condition, feeding behaviour and the accumulative unwellness of the caterpillars were monitored daily for a period of 6 days.

### In-Gel Trypsin Digestion and Mass Spectrometry Analysis

Heated and unheated samples were analysed by SDS-PAGE and the corresponding protein bands were excised from the gels using a clean scalpel blade for each sample to avoid cross-contamination. All excised gel bands were sent to Centre for Genomics Proteomics and Metabolomics (University of Auckland), for overnight in-gel trypsin digestion, followed by mass spectrometry protein identification using 10-fold diluted samples on the state-of-the-art Sciex Triple TOF 6600 LC-MS/MS spectrometer.

Detailed ProteinPilot MS/MS peptide summary and protein summary for each sample were generated at the end of each MS run. At the end of each LC-MS/MS run, the resulting MS data was used to search against Bacillus databases to generate a comprehensive ProteinPilot MS/MS peptide summary and a full-length protein summary for each sample.

Briefly, any MS/MS ion peak with a Confidence Level <30, or duplicate sequence modifications or elution time within 0.5 min was discarded in order to ensure a highly accurate non-redundant MS analysis with a dMass (difference between experimental and theoretical mass) between 1-10 ppm. All MS/MS peptide and precursor intensities, sequence coverage % and Confidence Level were checked for each potential protein candidate.

Online proteomics tools including BLAST (blastp: protein-protein blast), PROSITE (database of protein domains, families and functional sites) and SignalP (signal peptide cleavage site prediction) were used to analyse the MS results.

### Enzymatic Cleavage and Partial Purification of S-Layer Protein

A large-scale double-digestion proteolysis using lysozyme and mutanolysin, at 37° C. for 30 min, was carried out to remove full-length, stable, soluble and biologically-active S-layer protein from the bacterial cells, with partial purification confirmed by SDS-PAGE analysis (data not shown). A total of 24 proteolysis reactions were performed in order to provide purified S-layer protein for bioactivity assessment. This ˜24 mL of purified S-layer protein together with the removed peptidoglycan-layer was then concentrated down to ˜3 mL using a Vivaspin20 membrane filter concentrator with a MWCO of 3 kDa, and used in the DBM larvae bioassay to assess its bioactivity.

### Results

A typical time-course of protein expression profiles of Bl45 cell culture is illustrated in FIG. 17. A clear increase in expression levels of the protein species with a MW of ˜135 kDa and ˜120 kDa was observed, with peak amounts at 23 hr, and then expression gradually reduced over time. A protein species at ˜60 kDa was also expressed steadily from 23 hr onwards and slowly reduced by 96 hr. A ˜16 kDa protein species appeared gradually from 23 hr onwards to the end of the time-course study, where the intensity was the highest at 96 hr. A clear ˜90 kDa species could also been found at 96 hr. A large amount of other proteins were also expressed, although their expression levels were comparatively lower.

It appears that the highest protein expression level occurs at 23 hr after inoculation, which coincides with the beginning of the sporulation stage (24-48 hr). The increasing intensity of the ˜16 kDa species also coincides with the reducing levels of the higher MW species.

Cell cultures were harvested at 30 hours and at 72 hours, and aliquots were heated to give both heated and unheated samples for bioassay. In agreement with previous observations, as shown in FIG. 18, the unheated (UH) pellet samples exhibited a much higher biocontrol (additional 27-35%) compared to that observed in heated (H) pellets in both 72 hr and 96 hr bacterial biomass. This increased activity was not so evident in the samples from 30 hr cultures.

Notably, the 96 hr unheated pellet displayed the highest biocontrol with an unwellness of 88.89%, compared to 71.11% and 48.89% unwellness observed in 72 hr and 30 hr unheated pellets, respectively.

These data suggest that the older the Bl45 cell culture, up to 96 hr, the greater the insecticidal activity. The positive control (spinosad) showed very fast and effective control from Day 1 onwards, whereas the negative control (autoclaved water) showed reasonable activity (minimal control) up until Day 3 (8.89%), where by Day 6 the accumulative unwellness of caterpillars has increased to 28.89%, possibly due to starvation.

As can be seen in FIG. 19, a number of protein bands revealed in the unheated pellet samples were absent in the heated counterparts, most likely due to heat-induced protein degradation. These included the protein species at ˜135 kDa, ˜120 kDa, ˜90 kDa, ˜60 kDa and ˜18 kDa observed in the time-course study (see FIG. 17), as well as a number of protein species with a MW between 37 kDa to 50 kDa.

### In-Gel Trypsin Digest and Mass Spectrometry

Six protein bands (namely, species at 1) ˜16 kDa; 2) ˜90 kDa; 3) ˜60 kDa; 4) ˜12 kDa; 5) ˜120 kDa; and 6) ˜135 kDa) that were present in the unheated pellet samples, but absent in the heated counterparts, were excised from the protein gels, and subjected to protein identification by mass spectrometry.

One protein, comprising band 3, was identified as a common 60 kD GroEL chaperonin protein involved in protein folding.

The remaining five bands each belonged to the same protein candidate. Database and sequence analysis indicated this protein was the B. laterosporus S-layer protein identified in Example 6 above.

### Bioassay Using Partially Purified S-Layer Protein

As can be seen in FIG. 20, unheated, biologically-active S-layer protein exhibited a significant level of bioactivity from Day 2 onwards (S-layer protein-Unheated, 22.22%), whereas by Day 5 a strong 71.11% accumulative unwellness of caterpillars was observed. In contrast, the negative control group were largely unaffected (17.18%). The heated S-layer protein group also exhibited a certain level of unwellness by Day 5 (S-layer protein-Heated, 37.78%). Without wishing to be bound by any theory, applicants believe this results from the presence of other components of the Gram-positive bacterial peptidoglycan-layer, such as teichoic acids, lipoteichoic acids and possibly other bound carbohydrates, co-purifying with the S-layer protein.

In good agreement with previous bioassays, strong insecticidal activity and biocontrol was observed with the positive biological control comprising unheated Bl45 cells (Pellet-Unheated). Interestingly, unusually high biocontrol (82.22%) was observed with the heated counterpart (Pellet-Heated), however this was attributed to a lower temperature heat inactivation step of 88° C. being employed compared to the 100° C. heat inactivations previously used. Without wishing to be bound by any theory, the applicants expect that this lower temperature may not be sufficient to fully heat-inactivate the biomass, given the reported viability of B. laterosporus spores after heating at 85° C.

Also of note was the marginal biocontrol observed with the cell wall-free protoplasts (Protoplasts) over the first 3 days compared to the negative control. Biocontrol then increased to 40% on Day 5. Again without wishing to be bound by any theory, applicants believe the delay in bioactivity observed with the cell wall-free protoplasts is consistent with an expected delay in infection of target insects due to the lack of the proteolytically-removed S-layer proteins, which are postulated to facilitate host cell adhesion and/or cellular degradation of target insect cells.

### DISCUSSION

The toxicity of Bl45 toward DBM larvae, Plutella xylostella, was mainly located in the culture supernatant, indicating that the toxic components were excreted. A principal toxin has been identified as a surface layer (S-layer) protein with surface layer homology (SLH) domains. A potential adhesin-encoding gene and transporter-encoding gene were identified as putative accessory virulent determinants and located directly upstream and downstream to the surface layer protein-encoding gene.

Surface layer proteins are abundant in many bacteria and archaea (Engelhardt, 2007), and self-assemble into para-crystalline protein sheets onto the surface of microbial cells (Kern et al., 2011).

The S-layer protein-encoding gene in Bl45 appears to be highly conserved within Bl and other Brevibacillus species, but nevertheless has substantial differences to orthologues of related bacteria. The applicants predict, without wishing to be bound by any theory, that these differences might represent different host specificities or might represent the difference between insecticidal activity or no insecticidal activity.

### The Surface Layer Homology Domain

Surface layer proteins can anchor themselves to the bacterial cell wall by the SLH domain which non-covalently binds to the secondary cell wall polysaccharides (SCWPs) (Cava et al., 2004). Two putative SLH domains were detected in the putative S-layer encoding gene of Bl45. Additionally, the gene also shows sequence homology to the SLH structure sequence of Sap, a S-layer protein from Bacillus anthracis (Kern et al., 2011), a spore forming bacterium and the causal agent of anthrax. Sap forms crystalline arrays along the bacterial cell surface (Kern et al., 2010; Mesnage et al., 2000).

### The Glycoside Hydrolase Homologs; Chitinase and Cellobiohydrolase

Sequence homology of the S-layer protein from Bl45 to the structurally elucidated ChiW was detected. ChiW is a unique chitinase from Paenibacillus sp. str. FPU 7 (Itoh et al., 2016) with two active sites. It is expressed on the cell surface of Paenibacillus and contains three SLH domains. ChiW can hydrolyse a number of chitins, including crystalline chitin (Itoh et al., 2016). The S-layer encoding gene from Bl45 also showed sequence homology to the amino acid sequence of the structurally elucidated cellobiohydrolase from Clostridium thermocellum. Cellobiohydrolase is part of the cellulosome of C. thermocellum, a cellulase that can hydrolyse cell wall polysaccharides in plant cells (Guimaraes et al., 2002). It was proposed that cellobiohydrolase and endoglucanase families have a strong evolutionary relationship and could therefore be classified into a new family of glycoside hydrolases (Guimaraes et al., 2002). Glycoside hydrolases are enzymes that can hydrolyse glycosidic bonds in complex sugars, are very common in nature and can be found in the whole realm of life (Naumoff, 2011). Cellobiohydrolase, cellulase and chitinase are each members of the glycoside hydrolase superfamily.

However, the sequence identities of the S-layer protein to the structurally mapped ChiW chitinase and CelS cellbiohydrolase were very low, with 16% and 29% sequence identity, respectively, and with 8% and 3% query coverage, respectively.

Despite the low sequence identities of the chitinase and cellbiohydrolase homologs, the results suggest that the S-layer protein of Bl45 possesses domains that belong to the glycoside hydrolase superfamily.

The applicants expect, without wishing to be bound by any theory, that the S-layer protein has enzymatic activity capable of hydrolysing complex carbohydrates, such as chitin present in the body wall of the DBM caterpillars, and/or cellulose present in the cell walls of plants, such as the cabbage leaves used in the bioassays presented herein. This may explain the brown colouring and soft wet appearance of the dead DBM caterpillars treated with the concentrated quenched supernatant containing the S-layer protein, and the fact that cabbage leaves coated with the concentrated quenched supernatant turned brown and wet after 2-3 days of incubation and were covered in fungi by about 3-4 days after incubation.

### The 235 kDa Rhoptry Protein Domain

Sequence homology to the 235 kDa rhoptry protein family domain was identified in the putative S-layer encoding gene. The 235 kDa rhoptry protein family is part of a multigene family expressed in different strains of the protozoan Plasmodium species, the causative agent of malaria in mammals including humans (Khan et al., 2001). The 235 kDa rhoptry protein is part of the rhoptry organelle, located at the apex of the Plasmodium cell and plays an important role in host cell recognition, therefore reportedly enabling the subsequent invasion of Plasmodium merozoites into the host cell (Preiser et al., 2000). It binds to specific receptors on red blood cells, either the younger reticulocytes or normocytes depending on the strain of Plasmodium.

Without wishing to be bound by any theory, the conserved 235 kDa rhoptry protein domain identified within the S-layer encoding gene may represent a host cell recognition site, for example, for specific DBM-cells such as midgut epithelial cells or haemocytes within the haemolymph of the insect.

### The Putative Location and Function of the Surface Layer Proteins in Bl45 and B144

TEM results (data not shown) revealed that mature endospores of both Bl45 and B144 have similar structures and both contain a crust that is also similar in structure.

The applicants propose, without wishing to be bound by any theory, that the spore crust of Bl45 and B144 contains the toxic S-layer protein, a principal toxin of both strains.

Pieces of detached crust were observed by TEM in both strains (data not shown). The S-layer protein was identified in the culture supernatant of Bl45, which demonstrated that the protein is excreted into the environment. The protein also contains SLH-domains, which generally anchor to the cell surface and could facilitate anchorage to the spore coat surface.

This could also explain why unwashed spores of Bl45 suspended in water were still highly toxic to DBM larvae (Table 2), but 3× washed spores did not display any significant activity toward DBM larvae (Tables 4, 5 and 6). The transmission electron micrographs of both strains have shown that pieces of crust detach from the spore surface into the environment. Washing the spores repeatedly is therefore likely to remove a large proportion of the endospore crust.

### Heat Sensitivity Versus Heat Stability of Toxins Produced by BL45

Heat-sensitivity of culture supernatant samples was observed in four separate bioassays in which five different cultures were tested for insecticidal activity. The heated and unheated culture supernatant were analysed by SDS-PAGE in one of these bioassays. The heated culture supernatant contained a lane of smeared bands, unlike the unheated culture supernatant, which displayed a lane with primarily sharp bands. These results suggest that the proteins in the heated culture supernatant were degraded by the heat treatment. The loss of activity of the heated culture supernatant and the degradation of the proteins in this sample, highly suggest that toxins of proteinaceous nature of Bl45 are sensitive to heat.

The occasional heat-stability of insecticidal activity observed in the heated culture supernatant of some Bl45 cultures may be associated with a non-proteinaceous metabolite.

The observed loss of activity of the heated culture supernatant of a number of Bl45 cultures was likely caused by the degradation of heat-treated proteins, including the S-layer protein. It will be appreciated that the culture supernatant of Bl45 likely consists of a mixture of proteins and other components such as secondary metabolites, and that the presence of a proteinaceous insecticidal activity does not precule the production by Bl45 of one or more other toxins active against insects and possibly other organisms.

As shown in Example 8 above, mass spectrometry of five protein bands derived from a washed bacterial spore pellet in a second set of analyses also identified the S-layer protein. A crude protein extract containing the S-layer protein was obtained by the enzymatic treatment with lysozyme and mutanolysin of a washed pellet derived from a 96 hour spore culture. The protein extract was concentrated eight times using a Vivaspin 20 concentrator with a molecular cut off weight of 3 kDa. The S-layer protein fraction exhibited a high cumulative mortality of 71% among DBM larvae over five days. The heated S-layer protein fraction had a considerably reduced cumulative mortality of 38% after five days, suggesting that the S-layer protein fraction is heat sensitive in this case also.

As suggested above, based on the identification of putative glycosyl hydrolase domain, the S-layer protein is believed, without wishing to be bound by any theory, to facilitate the degradation of chitin present in invertebrates and/or degrade cellulose present in plant cell walls. This would allow vegetative cells and endospores access to nutrients, which in turn can trigger germination and propagation. The S-layer protein could therefore play an important role in determining the ecological niche of Bl45 and play an important role in promoting propagation and survival, in addition to its insecticidal activity.

### Bacterial Adhesins, Function, Homologs of the Putative Bl45 Adhesin and Potential Conserved Domains

Sequence homology analysis showed that the putative fimbriae/adhesin-encoding gene of Bl45 does not appear to be highly conserved in other Brevibacillus species. The gene appears to be mostly conserved in the bacterial genus Exiguobacterium. The best matching protein homolog to the putative adhesin-encoding gene was a hypothetical protein from Bacillus manliponensis, with a query cover of 86% and identity of 41%. The substantial significance of this match was indicated by the low E-value of 1×10⁻¹¹⁸.

The level of identity is relatively high, but sufficiently low to suggest that the adhesin-like protein is unique to Bl45, and may have a unique function. Without wishing to be bound by any theory, a function of the putative adhesin may be host cell recognition and adhesion to DBM host cells. The putative adhesin-encoding gene may therefore be part of a main virulence factor of Bl45 with regard to the DBM.

Bacterial Efflux Pumps, Function, Homologs of the Putative Bl45 efflux Pump and Potential Conserved Domains

Sequence homology to TolC from E. coli, the outer membrane efflux protein family (OEP), ST50 from S. enterica and the T1SS domain suggest that the putative transporter encoding gene from Bl45 encodes an outer membrane protein that is part of an efflux pump. Without wishing to be bound by any theory, this pump might be responsible for the excretion of the putative larvicidal S-layer protein and for the excretion of the putative accessory virulent fimbriae/adhesin-like protein from Bl45.

These examples clearly support the insecticidal efficacy of the S-layer polypeptides and accessory proteins identified herein, and support their use in the biological control of insect pests, whereby these insecticidal polypeptides have significant potential to provide agricultural and economic benefit.

### Example 9: Assessment of Insecticidal Activity

This example describes a mortality trial to assess the insecticidal activity of various Brevibacillus laterosporus derived compositions against Diamondback moth (Plutella xylostella, Lepidoptera: Plutellidae).

### Methods

### Bioassay

Circular segments were cut from pak choy using the bottom plate of a petri dish as a template. Outer leaves of smaller pak choy (5-6 leaves) were used. After cutting, the leaves were soaked for 60 min in filtered water with a small volume of peroxide solution to remove any bacteria that may otherwise cause rotting during the assay.

Leaf segments were placed on water agar in petri dishes, with the bottom of the leaf facing upwards. Segments were placed to ensure a seal was formed around the edge of the leaf segment to prevent larvae/insects from crawling underneath the leaf segment.

Diamondback moth larvae of 2^{nd} instar, from 2 mm to 4 mm in length, were gently placed on top of the leaf segment. 10 larvae were used per sample/plate, in accordance with experimental design.

Plates were sprayed within a Potters tower. Typically, 4 mL test and control samples were sprayed (at 5 psi to ensure good coverage) per plate as per experimental design.

After spraying, plates were transferred to a controlled climate environment (usually ˜88% humidity, ˜24° C., 8 hours dark), and observations were made in accordance with the experimental design. Typically, observations are made every 24 hours over the course of the trial.

Morbidity and mortality were recorded at each observation point, and photos were take to track eating habits and percentage of leaf damage, in addition to pathology where relevant.

### Test Samples

Test and control samples were prepared as follows:

Spinosad was used as a positive insecticide control, used as per the manufacturer's instructions.

Water alone (Water), and water with eNtomate™ surfactant (2.5 mL/L, Entomate) were used as negative controls.

Test samples comprised:
B. laterosporus NMI No. V12/001946 cells only, prepared from centrifuged cell culture from which the culture supernatant has been removed (Pellet Recon);
a cell-free culture supernatant prepared from B. laterosporus NMI No. V12/001946 cell culture (Supernatant);
a sub 3 kDa filtrate of B. laterosporus NMI No. V12/001946 culture extract (<3 kDa),
an autoclaved B. laterosporus NMI No. V12/001946 whole cell culture (Autoclaved WC); and
a B. laterosporus NMI No. V12/001946 whole cell culture (Whole Culture).

All test samples were prepared with eNtomate™ surfactant (2.5 mL/L).

Filtered samples were prepared using a 3 kDa Amicon Ultra cutoff filter (Merck).

Samples comprising B. laterosporus NMI No. V12/001946 culture or extracts were diluted to 1×10⁸ spores/mL with water, and 4 mL of diluted sample was sprayed onto each sample pak choy leaf disc.

Mortality % was assessed once per day over the three day trial.

### Results

As can be seen in FIG. 21, all samples either containing or derived from B. laterosporus NMI No. V12/001946 showed insecticidal activity against target lepidopterans above that of the negative controls.

Furthermore, all samples comprising components of the culture supernatant from B. laterosporus NMI No. V12/001946 cell culture had at least comparable, if not superior, insecticidal activity than B. laterosporus NMI No. V12/001946 cells alone: compare the mortality observed with each of Whole Culture, Autoclaved WC, <3 kDa, and Supernatant mortality with that observed with Pellet Recon.

Indeed, the Whole Culture sample, comprising culture supernatant in addition to viable B. laterosporus NMI No. V12/001946 had substantially greater insecticidal activity than that of B. laterosporus NMI No. V12/001946 cells alone. Furthermore, the cell-free culture supernatant had comparable insecticidal activity to B. laterosporus NMI No. V12/001946 cells alone—compare Supernatant to Pellet Recon. The toxicity observed with samples comprising B. laterosporus NMI No. V12/001946 culture supernatants or extracts exhibited contact action associated with secreted metabolites and/or the >3 kDa fraction. When viable B. laterosporus NMI No. V12/001946 cells were present, such samples also exhibited toxicity associated with viable cells, such that the presence of secreted metabolites or culture fractions did not interfere with toxicity modalities associated with viable cells.

These data establish that substantial insecticidal efficacy can be achieved using compositions comprising a culture supernatant or extracts from B. laterosporus NMI No. V12/001946, and indeed substantial efficacy can be achieved without viable B. laterosporus NMI No. V12/001946 cells being present.

### Example 10: Assessment of Insecticidal Activity

This example describes a mortality trial to assess the insecticidal activity of various Brevibacillus laterosporus derived compositions against Diamondback moth (Plutella xylostella, Lepidoptera: Plutellidae).

The mortality bioassay was performed as described in Example 9. Test and control samples were prepared as follows:

Spinosad was used as a positive insecticide control, used as per the manufacturer's instructions.

Water alone (Water), and water with eNtomate™ surfactant (2.5 mL/L, Entomate) were used as negative controls.

Test samples comprised:
B. laterosporus NMI No. V12/001944 cells only, prepared from centrifuged cell culture from which the culture supernatant has been removed (Pellet Recon);
a culture supernatant prepared from B. laterosporus NMI No. V12/001944 cell culture (Supernatant);
a sub 3 kDa filtrate of autoclaved B. laterosporus NMI No. V12/001944 culture extract (<3 kDa),
an autoclaved B. laterosporus NMI No. V12/001944 whole cell culture (Autoclaved WC); and
a B. laterosporus NMI No. V12/001944 whole cell culture (Whole Culture).

All test samples were prepared with eNtomate™ surfactant (2.5 mL/L).

Filtered samples were prepared using a 3 kDa Amicon Ultra cutoff filter (Merck).

Samples comprising B. laterosporus NMI No. V12/001944 culture or extracts were diluted to 1×10⁸ spores/mL with water, and 4 mL of diluted sample was sprayed onto each sample pak choy leaf disc.

Mortality % was assessed once per day over the four day trial.

### Results

As can be seen in FIG. 22, all samples either containing or derived from B. laterosporus NMI No. V12/001944 showed insecticidal activity against target lepidopterans above that of the negative controls.

Furthermore, all samples comprising components of the culture supernatant from B. laterosporus NMI No. V12/001944 cell culture had at least comparable, if not superior, insecticidal activity than B. laterosporus NMI No. V12/001944 cells alone: compare the mortality observed with each of Whole Culture, Autoclaved WC, <3 kDa, and Supernatant mortality with that observed with Pellet Recon.

Indeed, the Whole Culture sample, comprising culture supernatant in addition to viable B. laterosporus NMI No. V12/001944 had substantially greater insecticidal activity than that of B. laterosporus NMI No. V12/001944 cells alone. Furthermore, the cell-free culture supernatant had superior insecticidal activity to B. laterosporus NMI No. V12/001944 cells alone—compare Supernatant to Pellet Recon. The toxicity observed with samples comprising B. laterosporus NMI No. V12/001944 culture supernatants or extracts exhibited contact action associated with secreted metabolites and/or the >3 kDa fraction. When viable B. laterosporus NMI No. V12/001944 cells were present, such samples also exhibited toxicity associated with viable cells, such that the presence of secreted metabolites or culture fractions did not interfere with toxicity modalities associated with viable cells.

These data establish that substantial insecticidal efficacy can be achieved using compositions comprising a culture supernatant or extracts, such as secreted extracts or fractions comprising secreted metabolites from B. laterosporus NMI No. V12/001944, and indeed substantial pesticidal efficacy can be achieved without viable B. laterosporus NMI No. V12/001944 cells being present.

### PUBLICATIONS

Arnesano, F., Banci, L., Bertini, I., Mangani, S., & Thompsett, A. R. (2003). A redox switch in CopC: an intriguing copper trafficking protein that binds copper(I) and copper(II) at different sites. Proceedings of the National Academy of Sciences, 100(7), 3814-3819. doi:10.1073/pnas.0636904100.
Arnold, K., Bordoli, L., Kopp, J., & Schwede, T. (2006). The SWISS-MODEL workspace: a web-based environment for protein structure homology modelling. Bioinformatics, 22(2), 195-201. doi:10.1093/bioinformatics/bti770.
Bavro, V. N., Pietras, Z., Furnham, N., Perez-Cano, L., Fernandez-Recio, J., Pei, X. Y., Luisi, B. (2008). Assembly and channel opening in a bacterial drug efflux machine. Molecular Cell, 30(1), 114-121. doi:10.1016/j.molcel.2008.02.015.
Baxter, S. W., Badenes-Perez, F. R., Morrison, A., Vogel, H., Crickmore, N., Kain, W., Wang, P., Heckel, D. G. and Jiggins, C. D. (2011) Parallel evolution of Bacillus thuringiensis toxin resistance in Lepidoptera. Genetics October, 189, 675-679.
Biasini, M., Bienert, S., Waterhouse, A., Arnold, K., Studer, G., Schmidt, T., . . . Schwede, T. (2014). SWISS-MODEL: modelling protein tertiary and quaternary structure using evolutionary information. Nucleic Acids Research, 42(Web Server issue), W252-258. doi:10.1093/nar/gku340.
Cava, F., de Pedro, M. A., Schwarz, H., Henne, A., & Berenguer, J. (2004). Binding to pyruvylated compounds as an ancestral mechanism to anchor the outer envelope in primitive bacteria. Molecular Microbiology, 52(3), 677-690. doi:10.1111/j.1365-2958.2004.04011.x.
Engelhardt, H. (2007). Are S-layers exoskeletons? The basic function of protein surface layers revisited. Journal of Structural Biology, 160(2), 115-124. doi:10.1016/j.jsb.2007.08.003.
Faijes, M., Mars, A. E., & Smid, E. J. (2007). Comparison of quenching and extraction methodologies for metabolome analysis of Lactobacillus plantarum. Microbial Cell Factories, 6, 27. doi:10.1186/1475-2859-6-27.
Favret, M. E. and Yousten, A. A. (1985) Insecticidal activity of Bacillus laterosporus. J. Invertebrate Pathology 48, 195-203.
Ganesh et al., in prep.
Gohar, M., & Perchat, S. (2001). Sample preparation for beta-exotoxin determination in Bacillus thuringiensis cultures by reversed-phase high-performance liquid chromatography. Analytical Biochemistry, 298(1), 112-117. doi:10.1006/abio.2001.5373.
Griessl, M. H., Schmid, B., Kassler, K., Braunsmann, C., Ritter, R., Barlag, B., Muller, Y. A. (2013). Structural insight into the giant Ca2+-binding adhesin SiiE: implications for the adhesion of Salmonella enterica to polarized epithelial cells. Structure, 21(5), 741-752. doi:10.1016/j.str.2013.02.020.
Guan, H. H., Yoshimura, M., Chuankhayan, P., Lin, C. C., Chen, N. C., Yang, M. C., Chen, C. J. (2015). Crystal structure of an antigenic outer-membrane protein from Salmonella Typhi suggests a potential antigenic loop and an efflux mechanism. Scientific Reports, 5, 16441. doi:10.1038/srep16441.
Guex, N., Peitsch, M. C., & Schwede, T. (2009). Automated comparative protein structure modeling with SWISS-MODEL and Swiss-PdbViewer: a historical perspective. Electrophoresis, 30 Suppl 1, S162-173. doi:10.1002/elps.200900140.
Guimaraes, B. G., Souchon, H., Lytle, B. L., David Wu, J. H., & Alzari, P. M. (2002). The crystal structure and catalytic mechanism of cellobiohydrolase CelS, the major enzymatic component of the Clostridium thermocellum cellulosome. Journal of Molecular Biology, 320(3), 587-596.
Guo, S., Garnham, C. P., Karunan Partha, S., Campbell, R. L., Allingham, J. S., & Davies, P. L. (2013). Role of Ca2+ in folding the tandem beta-sandwich extender domains of a bacterial ice-binding adhesin. FEBS Journal, 280(22), 5919-5932. doi:10.1111/febs.12518.
Itoh, T., Hibi, T., Suzuki, F., Sugimoto, I., Fujiwara, A., Inaka, K., Kimoto, H. (2016). Crystal structure of chitinase ChiW from Paenibacillus sp. str. FPU-7 reveals a novel type of bacterial cell-surface-expressed multi-modular enzyme machinery. PLoS One, 11(12), e0167310. doi:10.1371/journal.pone.0167310.
Khan, S. M., Jarra, W., & Preiser, P. R. (2001). The 235 kDa rhoptry protein of Plasmodium (yoelii) yoelii: function at the junction. Molecular and Biochemical Parasitology, 117(1), 1-10.
Kern, J., & Schneewind, 0. (2010). BsIA, the S-layer adhesin of B. anthracis, is a virulence factor for anthrax pathogenesis. Molecular Microbiology, 75(2), 324-332. doi:10.1111/j.1365-2958.2009.06958.x.
Kern, J., Wilton, R., Zhang, R., Binkowski, T. A., Joachimiak, A., & Schneewind, 0. (2011). Structure of surface layer homology (SLH) domains from Bacillus anthracis surface array protein. Journal of Biological Chemistry, 286(29), 26042-26049. doi:10.1074/jbc.M111.248070.
Kiefer, F., Arnold, K., Kunzli, M., Bordoli, L., & Schwede, T. (2009). The SWISS-MODEL Repository and associated resources. Nucleic Acids Research, 37(Database issue), D387-392. doi:10.1093/nar/gkn750.
Lei, H. T., Bolla, J. R., Bishop, N. R., Su, C. C., & Yu, E. W. (2014). Crystal structures of CusC review conformational changes accompanying folding and transmembrane channel formation. Journal of Molecular Biology, 426(2), 403-411. doi:10.1016/j.jmb.2013.09.042.
Mesnage, S., Fontaine, T., Mignot, T., Delepierre, M., Mock, M., & Fouet, A. (2000). Bacterial SLH domain proteins are non-covalently anchored to the cell surface via a conserved mechanism involving wall polysaccharide pyruvylation. EMBO Journal, 19(17), 4473-4484. doi:10.1093/emboj/19.17.4473.
Naumoff, D. G. (2011). Hierarchical classification of glycoside hydrolases. Biochemistry (Mosc), 76(6), 622-635. doi:10.1134/S0006297911060022.
Palma, L., Munoz, D., Berry, C., Murillo, J., & Caballero, P. (2014). Bacillus thuringiensis toxins: an overview of their biocidal activity. Toxins (Basel), 6(12), 3296-3325. doi:10.3390/toxins6123296.
Pavkov, T., Egelseer, E. M., Tesarz, M., Svergun, D. I., Sleytr, U. B., & Keller, W. (2008). The structure and binding behavior of the bacterial cell surface layer protein SbsC. Structure, 16(8), 1226-1237. doi:10.1016/j.str.2008.05.012.
Preiser, P., Kaviratne, M., Khan, S., Bannister, L., & Jarra, W. (2000). The apical organelles of malaria merozoites: host cell selection, invasion, host immunity and immune evasion. Microbes and Infection, 2(12), 1461-1477.
Tabashnik, B. E., Cushing, N. L., Finson, N., Johnson, M. W. (1990) Field Development of Resistance to Bacillus thuringiensis in Diamondback Moth (Lepidoptera: Plutellidae). Journal of Economic Entomology, 83, 1671-1676.
Tabashnik, B. E., Liu, Y., Malvar, T., Heckel, D. G., Masson, L. and Ferre, J. (1998) Insect resistance to Bacillus thuringiensis: uniform or diverse? Phil. Trans. R. Soc. Land. B 29 October 353, 1751-1756.
Yonehara, R., Yamashita, E., & Nakagawa, A. (2016). Crystal structures of OprN and OprJ, outer membrane factors of multidrug tripartite efflux pumps of Pseudomonas aeruginosa. Proteins, 84(6), 759-769. doi:10.1002/prot.25022

As used in this specification, the words “comprise”, “comprises”, “comprising”, and similar words, are not to be interpreted in an exclusive or exhaustive sense. In other words, they are intended to mean “including, but not limited to”. When interpreting each statement in this specification that includes the term “comprise”, “comprises”, or “comprising”, features other than that or those prefaced by the term may also be present.

The entire disclosures of all applications, patents and publications cited above and below, if any, are herein incorporated by reference.

Where in the foregoing description reference has been made to integers or components having known equivalents thereof, those integers are herein incorporated as if individually set forth.

It should be noted that various changes and modifications to the presently preferred embodiments described herein will be apparent to those skilled in the art. Such changes and modifications may be made without departing from the spirit and scope of the invention and without diminishing its attendant advantages. It is therefore intended that such changes and modifications be included within the present invention.

The invention may also be said broadly to consist in the parts, elements and features referred to or indicated in the specification of the application, individually or collectively, in any or all combinations of two or more of said parts, elements or features.

Aspects of the invention have been described by way of example only, and it should be appreciated that variations, modifications and additions may be made without departing from the scope of the invention, for example when present the invention as defined in the indicative claims. Furthermore, where known equivalents exist to specific features, such equivalents are incorporated as if specifically referred in this specification.

1. An isolated, purified, recombinant or synthetic polypeptide selected from the group comprising:
a) a polypeptide comprising or consisting of the amino acid sequence depicted in any one of Sequence ID No.s 1 to 87;
b) a polypeptide comprising or consisting of the amino acid sequence corresponding to residues 89 to 322 of Sequence ID No. 1;
c) a polypeptide comprising or consisting of the amino acid sequence corresponding to residues 91 to 322 of Sequence ID No. 1;
d) a polypeptide comprising or consisting of the amino acid sequence corresponding to residues 147 to 322 of Sequence ID No. 1;
e) a polypeptide comprising or consisting of the amino acid sequence corresponding to residues 74 to 307 of Sequence ID No. 32;
f) a polypeptide comprising or consisting of the amino acid sequence corresponding to residues 76 to 307 of Sequence ID No. 32;
g) a polypeptide comprising or consisting of the amino acid sequence corresponding to residues 132 to 307 of Sequence ID No. 32;
h) a polypeptide comprising or consisting of the amino acid sequence corresponding to residues 89 to 322 of Sequence ID No. 58;
i) a polypeptide comprising or consisting of the amino acid sequence corresponding to residues 91 to 322 of Sequence ID No. 58;
j) a polypeptide comprising or consisting of the amino acid sequence corresponding to residues 147 to 322 of Sequence ID No. 58;
k) a polypeptide comprising or consisting of at least 10 contiguous amino acids from any one of a) to j) above;
l) a polypeptide comprising or consisting of at least 20 contiguous amino acids from any one of a) to j) above;
m) a polypeptide comprising or consisting of at least about 10 contiguous amino acids from any one of a) to l) above, wherein said polypeptide has glycoside hydrolase activity;
n) a polypeptide comprising or consisting of at least about 10 contiguous amino acids from any one of a) to m) above, wherein said polypeptide has chitinase activity or one or more chitinase domains;
o) a polypeptide having at least about 90% amino acid identity to any one of a) to n) above;
p) a polypeptide having at least about 95% amino acid identity to any one of a) to n) above;
q) any combination of any two or more of a) to p) above.

2. A method for controlling one or more pests, comprising the step of contacting the one or more pests with a pesticidally-effective amount of one or more polypeptides as claimed in claim 1 or a composition comprising one or more polypeptides as claimed in claim 1.

3. An expression construct comprising a nucleic acid encoding a polypeptide as claimed in claim 1, a vector comprising an expression construct comprising a nucleic acid encoding a polypeptide as claimed in claim 1, or a host cell comprising said expression construct or vector.

4. A composition, including an agricultural composition, comprising one or more of the polypeptides as claimed in claim 1, together with a carrier.

5. The composition as claimed in claim 4, wherein the composition comprises one or more isolated, purified, recombinant or synthetic polypeptide selected from the group comprising:
a) a polypeptide comprising or consisting of the amino acid sequence depicted in any one of Sequence ID Nos. 1 to 87;
b) a polypeptide comprising or consisting of the amino acid sequence corresponding to residues 89 to 322 of Sequence ID No. 1;
c) a polypeptide comprising or consisting of the amino acid sequence corresponding to residues 91 to 322 of Sequence ID No. 1;
d) a polypeptide comprising or consisting of the amino acid sequence corresponding to residues 147 to 322 of Sequence ID No. 1;
e) a polypeptide comprising or consisting of the amino acid sequence corresponding to residues 74 to 307 of Sequence ID No. 32;
f) a polypeptide comprising or consisting of the amino acid sequence corresponding to residues 76 to 307 of Sequence ID No. 32;
g) a polypeptide comprising or consisting of the amino acid sequence corresponding to residues 132 to 307 of Sequence ID No. 32;
h) a polypeptide comprising or consisting of the amino acid sequence corresponding to residues 89 to 322 of Sequence ID No. 58;
i) a polypeptide comprising or consisting of the amino acid sequence corresponding to residues 91 to 322 of Sequence ID No. 58;
j) a polypeptide comprising or consisting of the amino acid sequence corresponding to residues 147 to 322 of Sequence ID No. 58;
k) a polypeptide comprising or consisting of at least 10 contiguous amino acids from any one of a) to j) above;
l) a polypeptide comprising or consisting of at least 20 contiguous amino acids from any one of a) to j) above;
m) a polypeptide comprising or consisting of at least about 10 contiguous amino acids from any one of a) to l) above, wherein said polypeptide has glycoside hydrolase activity; n) a polypeptide comprising or consisting of at least about 10 contiguous amino acids from any one of a) to m) above, wherein said polypeptide has chitinase activity or one or more chitinase domains;
o) a polypeptide having at least about 90% amino acid identity to any one of a) to n) above;
p) a polypeptide having at least about 95% amino acid identity to any one of a) to n) above;
and additionally comprises one or more of the group comprising:
a) from about 1% to about 80% w/w cellular extract obtained from B. laterosporus NMI No. V12/001945; or
b) from about 1% to about 80% w/w of a composition comprising or derived from media in which B. laterosporus NMI No. V12/001945 is or has been grown; or
c) both a) and b) above.

6. The composition as claimed in claim 4, wherein the composition comprises an extract or composition enriched in or to which has been added a sub-3 kDa fraction from B. laterosporus NMI No. V12/001945 or a culture thereof.

7. The composition as claimed in claim 5, wherein the extract or composition enriched in or to which has been added a sub-3 kDa fraction from B. laterosporus NMI No. V12/001945 or a culture thereof is substantially non-proteinaceous.

8. The composition as claimed in claim 4, wherein the composition comprises one or more of the group comprising:
a) from about 1% to about 80% w/w cellular extract obtained from B. laterosporus NMI No. V12/001946; or
b) from about 10% to about 80% w/w of a composition comprising or derived from media in which B. laterosporus NMI No. V12/001946 is or has been grown; or
c) from about 1% to about 80% w/w cellular extract obtained from B. laterosporus NMI No. V12/001944; or
d) from about 1% to about 80% w/w of a composition comprising or derived from media in which B. laterosporus NMI No. V12/001944 is or has been grown; or
e) any combination of two or more of any of a) to d) above.

9. The composition as claimed in claim 4, wherein the composition comprises one or more of the group comprising:
a) an extract or composition enriched in or to which has been added a sub-3 kDa fraction from B. laterosporus NMI No. V12/001946 or a culture thereof;
b) an extract or composition enriched in or to which has been added a sub-3 kDa fraction from B. laterosporus NMI No. V12/001945 or a culture thereof; or
c) an extract or composition enriched in or to which has been added a sub-3 kDa fraction from B. laterosporus NMI No. V12/001944 or a culture thereof; or
d) any combination of two or more of any of a) to c) above.

10. The composition as claimed in claim 4, wherein one or more of the sub-3 kDa fractions is substantially non-proteinaceous.

11. The composition as claimed in claim 4, wherein the composition is formulated as a water dispersible granule.

12. A composition formulated as a water dispersible granule (WDG) comprising one or more polypeptides as claimed in claim 1, together with one or more of the following:
a) from about 2% to about 80% w/w viable B. laterosporus NMI No. V12/001944, and/or B. laterosporus NMI No. V12/001945, and/or B. laterosporus NMI No. V12/001946;
b) from about 2% to about 80% w/w cellular extract obtained from B. laterosporus NMI No. V12/001944, and/or B. laterosporus NMI No. V12/001945, and/or B. laterosporus NMI No. V12/001946;
c) from about 2% to about 80% w/w of a composition comprising or derived from media in which B. laterosporus NMI No. V12/001944, and/or B. laterosporus NMI No. V12/001945, and/or B. laterosporus NMI No. V12/001946 is or has been grown;
d) from about 1% to about 50% w/w one or more wetting agent;
e) from about 1% to about 50% w/w one or more dispersant;
f) from about 2% to about 50% w/w one or more humectant or agent to control water activity;
g) from about 0% to about 50% w/w one or more protectants;
h) from about 0% to about 50% w/w one or more nutrients or mixture thereof;
i) from about 5% to about 80% w/w one or more filler;
j) from about 0% to about 20% w/w one or more antioxidant or UV radiation protectant;
k) from about 1% to about 50% w/w one or more binding agent;
l) from about 0% to about 50% one or more disintegrating agent;
m) from about 0% to about 10% w/w water;
n) any combination of two or more of any of a) to m) above.

13. The composition as claimed in claim 1 wherein the composition comprises one or more of piperonyl butoxide, safrole, apiole, parsley oil, and dillapiole.

14. The composition as claimed in claim 1 wherein the composition is a synergistic composition comprising dillapiole.

15. A method of treating or protecting a plant or its surroundings, and/or plant derived materials, from a pest or pest infestation wherein the method comprises applying to the plant or its surroundings one or more polypeptides as claimed in claim 1, and/or applying to the plant or its surroundings a composition as claimed in claim 1.

16. A method of controlling a pest or pest population and/or preventing a pest infestation, the method comprising contacting the pest or pest population with, or applying to a surface, an effective amount of one or more polypeptides as claimed in claim 1.

17. The method as claimed in claim 16, wherein the pest is or the pest population comprises one or more insect pests.

18. The method as claimed in claim 16, wherein the pest is or the pest population comprises one or more nematodes.

19. A method of preparing a pesticidal or insecticidal composition, the method comprising
a) optionally growing a culture of B. laterosporus NMI No. V12/001944, and/or B. laterosporus NMI No. V12/001945, and/or B. laterosporus NMI No. V12/001946;
b) providing a cellular extract obtained from B. laterosporus NMI No. V12/001944, and/or B. laterosporus NMI No. V12/001945, and/or B. laterosporus NMI No. V12/001946, and/or a composition comprising or derived from media in which B. laterosporus NMI No. V12/001944, and/or B. laterosporus NMI No. V12/001945, and/or B. laterosporus NMI No. V12/001946, is or has been grown, wherein the cellular extract or composition comprises one or more of the polypeptides as claimed in claim 1;
c) admixing the cellular extract and/or composition with one or more agriculturally-acceptable carriers to provide the insecticidal composition.

20. A method of preparing a pesticidal or insecticidal composition, the method comprising:
growing one or more cells comprising a nucleic acid encoding one or more isolated, purified, recombinant or synthetic polypeptide selected from the group comprising:
a) a polypeptide comprising or consisting of the amino acid sequence depicted in any one of Sequence ID Nos. 1 to 87;
b) a polypeptide comprising or consisting of the amino acid sequence corresponding to residues 89 to 322 of Sequence ID No. 1;
c) a polypeptide comprising or consisting of the amino acid sequence corresponding to residues 91 to 322 of Sequence ID No. 1;
d) a polypeptide comprising or consisting of the amino acid sequence corresponding to residues 147 to 322 of Sequence ID No. 1;
e) a polypeptide comprising or consisting of the amino acid sequence corresponding to residues 74 to 307 of Sequence ID No. 32;
f) a polypeptide comprising or consisting of the amino acid sequence corresponding to residues 76 to 307 of Sequence ID No. 32;
g) a polypeptide comprising or consisting of the amino acid sequence corresponding to residues 132 to 307 of Sequence ID No. 32;
h) a polypeptide comprising or consisting of the amino acid sequence corresponding to residues 89 to 322 of Sequence ID No. 58;
i) a polypeptide comprising or consisting of the amino acid sequence corresponding to residues 91 to 322 of Sequence ID No. 58;
j) a polypeptide comprising or consisting of the amino acid sequence corresponding to residues 147 to 322 of Sequence ID No. 58;
k) a polypeptide comprising or consisting of at least 10 contiguous amino acids from any one of a) to j) above;
l) a polypeptide comprising or consisting of at least 20 contiguous amino acids from any one of a) to j) above;
m) a polypeptide comprising or consisting of at least about 10 contiguous amino acids from any one of a) to l) above, wherein said polypeptide has glycoside hydrolase activity;
n) a polypeptide comprising or consisting of at least about 10 contiguous amino acids from any one of a) to m) above, wherein said polypeptide has chitinase activity or one or more chitinase domains;
o) a polypeptide having at least about 90% amino acid identity to any one of a) to n) above;
p) a polypeptide having at least about 95% amino acid identity to any one of a) to n) above;
or one or more host cells as claimed in claim 4, under conditions suitable for the expression of said one or more polypeptides;
a) providing a cellular extract from said one or more cells or one or more host cells, or a composition comprising or derived from media in which said one or more cells or host cells is or has been grown, wherein the cellular extract or composition comprises one or more of the polypeptides as claimed in claim 1;
b) admixing the cellular extract and/or composition with one or more agriculturally-acceptable carriers to provide the insecticidal composition.

21. The method of claim 19 wherein the composition is formulated as a water dispersible granule.
